(19) United States
(12) Patent Application Publication
(10) Pub. No.: US 2026/0226404 A1
(43) Pub. Date: Aug. 6, 2026

(54) METHOD FOR THE FERMENTATIVE PRODUCTION OF GUANIDINOACETIC ACID USING A MICROORGANISM COMPRISING A HETEROLOGOUS L-THREONINE 3-DEHYDROGENASE GENE (TDH) AND A GLYCINE C-ACETYLTRANSFERASE GENE (KBL)

(71) Applicant: Evonik Operations GmbH, Essen (DE)

(72) Inventors: Steffen Schaffer, Herten (DE); Martina Carrillo Camacho, Hanau (DE); Frank Schneider, Halle (DE); Mirja Wessel, Bochum (DE)

(73) Assignee: Evonik Operations GmbH, Essen (DE)

(21) Appl. No.: 19/151,363

(22) PCT Filed: Jan. 30, 2024
(86) PCT No.: PCT/EP2024/052187

§ 371 Date: Jul. 28, 2025

(30) Foreign Application Priority Data
Feb. 1, 2023 (EP) 23154397.6

(51) Int. Cl. C12N 1/20; C12N 9/00; C12N 9/04; C12N 9/10; C12P 13/04; C12R 1/15; C12R 1/38

(52) U.S. Cl. CPC C12N 1/20; C12N 9/0006; C12N 9/1007; C12N 9/1029; C12N 9/93; C12P 13/04; C12Y 101/01103; C12Y 201/01002; C12Y 201/03003; C12Y 201/04001; C12Y 203/01029; C12Y 403/02001; C12Y 603/04005; C12Y 603/04016; C12R 2001/15; C12R 2001/38

(57) ABSTRACT

The present application is directed to a microorganism containing at least one heterologous gene coding for a L-arginine:glycine amidinotransferase (AGAT), at least one heterologous L-threonine 3-dehydrogenase gene (tdh), and at least one heterologous glycine C-acetyltransferase (2-amino-3-oxobutanoate coenzyme A ligase) gene (kbl). Also disclosed is a method for the fermentative production of guanidinoacetic acid (GAA) using such microorganism. The disclosure also relates to a method for the fermentative production of creatine.

The present invention concerns a microorganism comprising at least one heterologous gene coding for a L-arginine:glycine amidinotransferase (AGAT) and at least one heterologous L-threonine 3-dehydrogenase gene (tdh) and at least one heterologous glycine C-acetyltransferase (2-amino-3-oxobutanoate coenzyme A ligase) gene (kbl) and a method for the fermentative production of guanidinoacetic acid (GAA) using such microorganism. The present invention also relates to a method for the fermentative production of creatine.

GAA is a colorless crystalline organic compound used as animal feed additive (e.g. WO2005120246 A1 and US2011257075 A1). GAA is a natural precursor of creatine (e.g. Humm et al., Biochem. J. (1997) 322, 771-776). Therefore, the supplementation of GAA allows for an optimal supply of creatine in the organism.

In biological systems GAA and ornithine are formed from arginine and glycine as starting materials by the catalytic action of an L-arginine:glycine-amidinotransferase (AGAT; EC 2.1.4.1). The reaction is depicted in FIG. 1. This reaction is also the first step in creatine biosynthesis.

Guthmiller et al. (J Biol Chem. 1994 Jul. 1; 269(26):17556-60) have characterized a rat kidney AGAT by cloning and heterologously expressing the enzyme in Escherichia coli (E. coli). Muenchhoff et al. (FEBS Journal 277 (2010) 3844-3860) report the first characterization of an AGAT from a prokaryote also by cloning and heterologously expressing the enzyme in E. coli.

A microorganism capable of producing guanidinoacetic acid (GAA) was published by Zhang et al. (ACS Synth. Biol. 2020, 9, 2066-275). They designed a reconstituted the ornithine cycle in E. coli by introducing a heterologous AGAT from different species (e.g., Homo sapiens, Cylindrospermopsis raciborskii, Moorea producens) and by introducing a citrulline synthesis module (e.g. ovexpression of carAB, argF and argl) and an arginine synthesis module (e.g. overexpression of argG, argH; introduction of aspA) into E. coli.

Schneider and Jankowitsch (WO2021122400 A1) propose a method to produce GAA using a microorganism having gene coding for a protein having the function of an L-arginine:glycine amidinotransferase and an increased carbamoyl phosphate synthase. The carbamoyl phosphate is an important precursor for the biosynthesis of GAA but also for L-arginine and other compounds. Furthermore, Wang et al. (Applied Microbiology and Biotechnology, 2021, vol. 105, pp. 3265-3276; https://doi.org/10.1007/s00253-021-11242-w) underlined among other that carbamoyl phosphate is essential for L-arginine production also in Corynebacterium sp.

To increase the production of GAA using a microorganism an intracellular high amount of the start materials arginine and/or glycine are necessary.

One approach to increase L-arginine concentrations in the cell is to prevent L-arginine exportation as proposed by Jankowitsch et al. (WO2022243116 A1). To achieve this these authors disclose a microorganism essentially comprising a heterologous AGAT (EC 2.1.4.1) gene, an overexpressed carAB operon, a deletion of the argR regulator gene and an inactivation or deletion of a gene encoding a protein having the function of arginine exporter as well as an inactivation or deletion of gene coding for the respective translational activator.

Glyoxylate may be used as starting material for the formation of glycine in the presence of an amino donor, such as amino acids, and a glyoxylate transaminase. Glyoxylate transaminases catalyses the transfer of an amino group from an amino acid to glyoxylate. The products of this transfer are glycine and the corresponding α-keto acid. In order to increase the intracellular glycine concentration Schneider and Jankowitsch (WO2022008280 A1) propose providing the microorganism with a glyoxylate aminotransferase gene in addition to an AGAT gene and demonstrated that, in the presence of the enzymatic AGAT activity, the combination of increased activity of a glyoxylate aminotransferase and increased ability to produce L-arginine improves GAA production.

Glycine can also be produced from threonine via two distinct routes (FIG. 2). The first route is catalyzed by a low-specificity L-threonine aldolase [EC 4.1.2.48]/L-threonine aldolase ([EC 4.1.2.5], Lta), an enzyme which cleaves L-threonine directly into glycine and acetaldehyde. The second route is a two-step process. In the first step L-threonine is reacted to 2-amino-3-ketobutyrate (2-amino-3-ketobutanoate) catalyzed by an L-threonine 3-dehydrogenase ([EC 1.1.1.103], also called threonine dehydrogenase (Tdh). In the second step 2-amino-3-ketobutyrate is cleaved into glycine and acetyl-CoA by the catalytic action of a glycine C-acetyltransferase [EC 2.3.1.19](alternative name: 2-amino-3-ketobutyrate coenzyme A ligase or 2-amino-3-oxobutanoate coenzyme A ligase, Kbl).

Smirnov and Kotliarova (WO2019130723 A1) propose using the second route and disclose a method for producing glycine by fermentation of a bacterium that has been modified to overexpress a gene encoding a protein having L-threonine 3-dehydrogenase activity (tdh) and a gene encoding a protein having 2-amino-3-oxobutanoate coenzyme A ligase activity (kbl).

The object of the present invention is to provide a microorganism transformed to be capable of producing GAA and which has a further way to provide glycine as starting material of the GAA biosynthesis and a process for producing GAA using such microorganism.

The problem is solved by a microorganism having an increased ability to produce L-arginine from L-ornithine compared with the ability of a wildtype microorganism or of a non-modified microorganism and comprising at least one heterologous gene coding for a protein having the function of a L-arginine:glycine amidinotransferase and comprising at least one heterologous gene encoding a protein having L-threonine 3-dehydrogenase activity (EC 1.1.1.103) and at least one heterologous gene encoding a protein having a glycine C-acetyltransferase activity (EC 2.3.1.29).

A heterologous gene means that the gene has been inserted into a host organism which does not naturally have this gene. Insertion of the heterologous gene in the host is performed by recombinant DNA technology. Microorganisms that have undergone recombinant DNA technology are called transgenic, genetically modified or recombinant. A heterologous protein means a protein that is not naturally occurring in the microorganism.

Proteins having the function of an L-arginine:glycine amidinotransferase (AGAT) belong to the amidinotransferase family. The amidinotransferase family comprises glycine (EC:2.1.4.1) and inosamine (EC:2.1.4.2) amidinotransferases, enzymes involved in creatine and streptomycin biosynthesis respectively. This family also includes arginine deiminases, EC:3.5.3.6. These enzymes catalyse the reaction:

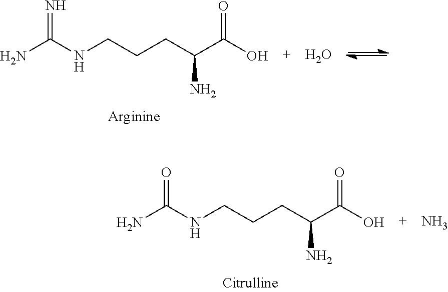

Also found in this family is the Streptococcus anti-tumor glycoprotein. Enzymes or proteins with an L-arginie:glycine-amidinotransferase (AGAT) activity are also described to possess a conserved domain that belongs to the PFAM Family: Amidinotransferase (PF02274) (Marchler-Bauer A et al. (2017), “CDD/SPARCLE: functional classification of proteins via subfamily domain architectures.”, Nucleic Acids Res. 45(D1):D200-D203.) as described also in the following publications: Pissowotzki K et al., Mol Gen Genet 1991; 231:113-123 (PUBMED:1661369 EPMC:1661369); D'Hooghe I et al., J Bacteriol 1997; 179:7403-7409 (PUBMED:9393705 EPMC:9393705); Kanaoka M et al., Jpn J Cancer Res 1987; 78:1409-1414 (PUBMED:3123442 EPMC:3123442).

In the context of the present invention, a microorganism having an increased ability to produce L-arginine means a microorganism producing L-arginine in excess of its own need. Examples for such L-arginine producing microorganisms are e.g. C. glutamicum ATCC 21831 or those disclosed by Park et al. (NATURE COMMUNICATIONS|DOI: 10.1038/ncomms5618) or by Ginesy et al. (Microbial Cell Factories (2015) 14:29).

In the microorganism according to the present invention the increased ability to produce L-arginine from L-ornithine compared with the ability of the wildtype microorganism or of a non-modified microorganism may be effected by an inactivated or deleted gene coding for an arginine responsive repressor protein ArgR and/or an overexpressed gene coding for an enzyme having the function of a carbamoylphosphate synthase (EC 6.3.4.16, CarAB), and/or at least one or more overexpressed genes selected from the group consisting of a gene (e.g. argF/argF2/argl) coding for a protein having the function of an ornithine carbamoyltransferase (EC 2.1.3.3), a gene (e.g. argG) coding for a protein having the function of an argininosuccinate synthetase (E.C. 6.3.4.5), and a gene (e.g. argH) coding for a protein having the function of an argininosuccinate lyase (E.C. 4.3.2.1).

In order to obtain relatively high L-arginine concentrations in the cell it is necessary to prevent L-arginine exportation. The amino acid exporter LysE counteracts the intracellular arginine concentration and reduces the substrate availability by efficiently transporting the substrate arginine from the cell. In addition, the citrulline from arginine biosynthesis is also secreted into the medium by an active LysE exporter. LysE is regulated by the transcriptional activator LysG (Bellmann, A., et al. (2001). “Expression control and specificity of the basic amino acid exporter LysE of Corynebacterium glutamicum.” Microbiology (Reading) 147(Pt 7): 1765-1774).

In a particular embodiment of the present invention a gene coding for protein having the function of an arginine exporter and, optionally, a gene coding for its transcriptional activator in the microorganism of the present invention is inactivated or deleted.

In a more particular embodiment of the present invention the lysEG gene/operon coding for a protein having the function of an arginine exporter LysE, and for its transcriptional activator LysG in the microorganism of the present invention is inactivated or deleted.

In the microorganism of the present invention the gene encoding a protein having L-threonine 3-dehydrogenase (Tdh) activity may further be overexpressed.

In the microorganism of the present invention also the gene encoding a protein having a glycine C-acetyltransferase (Kbl) activity may further be overexpressed.

Overexpression of a gene is generally achieved by increasing the copy number of the gene and/or by functionally linking the gene with a strong promoter and/or by enhancing the ribosomal binding site and/or by codon usage optimization of the start codon or of the whole gene or a combination comprising a selection or all methods mentioned above.

In particular, overexpression of a gene can be achieved by increasing the copy number of the gene and/or by an enhancement of regulatory factors, e.g. by functionally linking the gene with a strong promoter and/or by enhancing the ribosomal binding site and/or by codon usage optimization of the start codon or of the whole gene. The enhancement of such regulatory factors which positively influence gene expression can, for example, be achieved by modifying the promoter sequence upstream of the structural gene in order to increase the effectiveness of the promoter or by completely replacing said promoter with a more effective or a so-called strong promoter. Promoters are located upstream of the gene. A promoter is a DNA sequence consisting of about 40 to 50 base pairs and which constitutes the binding site for an RNA polymerase holoenzyme and the transcriptional start point, whereby the strength of expression of the controlled polynucleotide or gene can be influenced. Generally, it is possible to achieve an overexpression or an increase in the expression of genes in bacteria by selecting strong promoters, for example by replacing the original promoter with strong, native (originally assigned to other genes) promoters or by modifying certain regions of a given, native promoter (for example its so-called −10 and −35 regions) towards a consensus sequence, e.g. as taught by M. Patek et al. (Microbial Biotechnology 6 (2013), 103-117) for C. glutamicum. An example for a “strong” promoter is the superoxide dismutase (sod) promoter (“Psod”; Z. Wang et al., Eng. Life Sci. 2015, 15, 73-82). A “functional linkage” is understood to mean the sequential arrangement of a promoter with a gene, which leads to a transcription of the gene.

The genetic code is degenerated which means that a certain amino acid may be encoded by a number of different triplets. The term codon usage refers to the observation that a certain organism will typically not use every possible codon for a certain amino acid with the same frequency. Instead an organism will typically show certain preferences for specific codons meaning that these codons are found more frequently in the coding sequence of transcribed genes of an organism. If a certain gene foreign to its future host, i.e. from a different species, should be expressed in the future host organism the coding sequence of said gene should then be adjusted to the codon usage of said future host organism (i.e. codon usage optimization).

The microorganism of the present invention may belong to the genus Corynebacterium, preferably Corynebacterium glutamicum (C. glutamicum), or to the genus Enterobacteriaceae, preferably Escherichia coli (E. coli), or to the genus Pseudomonas, preferably Pseudomonas putida (P. putida).

In Corynebacterium glutamicum the gene coding for the protein having the function of an arginine exporter is lysE and the gene coding for the transcriptional activator is lysG. In Escherichia coli the gene coding for the protein having the function of an arginine exporter is argO (ybjE). In Pseudomonas putida the protein having the function of an arginine exporter is lysE.

The protein having the function of an L-arginine:glycine amidinotransferase (AGAT) in the microorganism of the present invention may comprise an amino acid sequence which is at least 80% identical, preferably at least 90% identical to the amino acid sequence according to SEQ ID NO: 44. In a further embodiment of the present invention the amino acid sequence of the L-arginine:glycine amidinotransferase is identical to amino acid sequence according to SEQ ID NO: 44.

The protein having L-threonine 3-dehydrogenase activity in the microorganism of the present invention may comprise an amino acid sequence which is at least 50-80% identical to the amino acid sequence according to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, or SEQ ID NO: 9. In a further embodiment, the protein having L-threonine 3-dehydrogenase activity in the microorganism of the present invention may comprise an amino acid sequence which is identical to one of these sequences.

The protein having a glycine C-acetyltransferase activity in the microorganism of the present invention may comprise an amino acid sequence which is at least 50-80% identical to the amino acid sequence according to SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, or SEQ ID NO: 18. In a further embodiment, the protein having glycine C-acetyltransferase activity in the microorganism of the present invention may comprise an amino acid sequence which is identical to one of these sequences.

The above-mentioned problem is further solved by a method for the fermentative production of guanidino acetic acid (GAA), comprising the steps of cultivating the microorganism according to the present invention as defined above in a suitable medium and accumulating GAA in the medium to form a GAA containing fermentation broth.

The method of the present invention may further comprise the step of isolating GAA from the fermentation broth.

The method according to the present invention may further comprise the step of drying and/or granulating the GAA containing fermentation broth.

The present invention further concerns a microorganism as defined above, further comprising a gene coding for an enzyme having the activity of a guanidinoacetate N-methyltransferase (EC: 2.1.1.2). Preferably, the gene coding for an enzyme having the activity of a guanidinoacetate N-methyltransferase is overexpressed.

The present invention also concerns a method for the fermentative production of creatine, comprising the steps of cultivating the microorganism according to the present invention comprising a gene coding for an enzyme having the activity of a guanidinoacetate N-methyltransferase in a suitable medium and accumulating creatine in the medium to form a creatine containing fermentation broth.

Preferably, the method further comprises isolating creatine from the creatine containing fermentation broth. creatine may be extracted from fermentation broth by isoelectric point method and/or ion exchange method. Alternatively, creatine can be further purified by a method of recrystallization in water.

### BRIEF DESCRIPTION OF THE FIGURES

FIG. 1: Reaction of L-arginine and glycine under the catalytic action of an L-arginine:glycine-amidinotransferase (AGAT; EC 2.1.4.1) to yield L-ornithine and GAA.

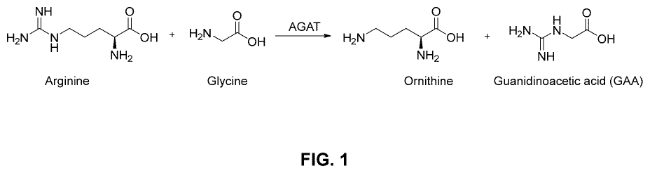

FIG. 2: Schematic depiction of the two distinct routes from L-threonine to glycine. Abbreviations: Tdh—L-threonine 3-dehydrogenase [EC 1.1.1.103], Kbl—glycine C-acetyltransferase [EC 2.3.1.19](alternative name: 2-amino-3-ketobutyrate coenzyme A ligase), Lta—low-specificity L threonine aldolase [EC 4.1.2.48] or L-threonine aldolase [4.1.2.5].

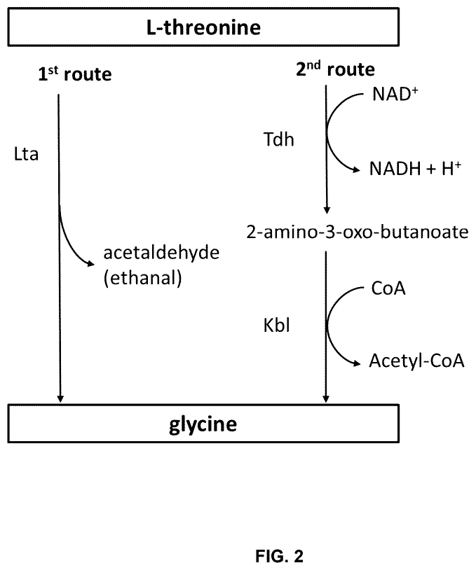

### BRIEF DESCRIPTION OF THE SEQUENCES

SEQ ID NO: 1 Amino acid sequence of an L-threonine 3-dehydrogenase (Tdh; EC 1.1.1.103) from Aliivibrio fischeri (Tdh_Afi) (Example 1).

SEQ ID NO: 2 Amino acid sequence of an L-threonine 3-dehydrogenase (Tdh; EC 1.1.1.103) from Bdellovibrio sp. (Tdh_Bds) (Example 1).

SEQ ID NO: 3 Amino acid sequence of an L-threonine 3-dehydrogenase (Tdh; EC 1.1.1.103) from Bos taurus (Tdh(*S51M)_Bot) (Example 1).

SEQ ID NO: 4 Amino acid sequence of an L-threonine 3-dehydrogenase (Tdh; EC 1.1.1.103) from Caulobacter sp. 17J65-9 (Tdh_Cs17) (Example 1).

SEQ ID NO: 5 Amino acid sequence of an L-threonine 3-dehydrogenase (Tdh; EC 1.1.1.103) from Cupriavidus necator H16 (Tdh_Re:H16) (Example 1).

SEQ ID NO: 6 Amino acid sequence of an L-threonine 3-dehydrogenase (Tdh; EC 1.1.1.103) from Corynebacterium xerosis (Tdh_Cx) (Example 1).

SEQ ID NO: 7 Amino acid sequence of an L-threonine 3-dehydrogenase (Tdh; EC 1.1.1.103) from Chlamydia sp. 32-24 (Tdh_Ch32) (Example 1).

SEQ ID NO: 8 Amino acid sequence of an L-threonine 3-dehydrogenase (Tdh; EC 1.1.1.103) from Cyanobacteria bacterium isolate (MAG with Accession No. JAIEMW010000004.1) (Tdh_Cyb) (Example 1).

SEQ ID NO: 9 Amino acid sequence of an L-threonine 3-dehydrogenase (Tdh; EC 1.1.1.103) from Escherichia coli K12 (Tdh_Ec) (Example 1).

SEQ ID NO: 10 Amino acid sequence of a glycine C-acetyltransferase (Kbl, EC 2.3.1.29) (alternative name: 2-amino-3-ketobutyrate coenzyme A ligase) from Aliivibrio fischeri (Kbl_Afi) (Example 1).

SEQ ID NO: 11 Amino acid sequence of a glycine C-acetyltransferase (Kbl, EC 2.3.1.29) (alternative name: 2-amino-3-ketobutyrate coenzyme A ligase) from Bdellovibrio sp. (Kbl_Bds) (Example 1).

SEQ ID NO: 12 Amino acid sequence of a glycine C-acetyltransferase (Kbl, EC 2.3.1.29) (alternative name: 2-amino-3-ketobutyrate coenzyme A ligase) from Bos taurus (Kbl(*Q21M)_Bot) (Example 1).

SEQ ID NO: 13 Amino acid sequence of a glycine C-acetyltransferase (Kbl, EC 2.3.1.29) (alternative name: 2-amino-3-ketobutyrate coenzyme A ligase) from Caulobacter sp. 17J65-9 (Kbl_Cs17) (Example 1).

SEQ ID NO: 14 Amino acid sequence of a glycine C-acetyltransferase (Kbl, EC 2.3.1.29) (alternative name: 2-amino-3-ketobutyrate coenzyme A ligase) from Cupriavidus necator H16 (Kbl_Re:H16) (Example 1).

SEQ ID NO: 15 Amino acid sequence of a glycine C-acetyltransferase (Kbl, EC 2.3.1.29) (alternative name: 2-amino-3-ketobutyrate coenzyme A ligase) from Corynebacterium xerosis (Kbl_Cx) (Example 1).

SEQ ID NO: 16 Amino acid sequence of a glycine C-acetyltransferase (Kbl, EC 2.3.1.29) (alternative name: 2-amino-3-ketobutyrate coenzyme A ligase) from Chlamydia sp. 32-24 (Kbl_Ch32) (Example 1).

SEQ ID NO: 17 Amino acid sequence of a glycine C-acetyltransferase (Kbl, EC 2.3.1.29) (alternative name: 2-amino-3-ketobutyrate coenzyme A ligase) from Cyanobacteria bacterium isolate (MAG with Accession No. JAIEMW010000004.1) (Kbl_Cyb) (Example 1).

SEQ ID NO: 18 Amino acid sequence of a glycine C-acetyltransferase (Kbl, EC 2.3.1.29) (alternative name: 2-amino-3-ketobutyrate coenzyme A ligase) from Escherichia coli K12 (Kbl_Ec) (Example 1).

SEQ ID NO: 19 DNA sequence of the plasmid pXMJ19[kbl_Afi tdh_Afi](Example 1).

SEQ ID NO: 20 DNA sequence of the plasmid pXMJ19[tdh_Bds kbl_Bds](Example 1).

SEQ ID NO: 21 DNA sequence of the plasmid pXMJ19[kbl(*Q21M)_Bot tdh(*S51M)_Bot](Example 1).

SEQ ID NO: 22 DNA sequence of the plasmid pXMJ19[kbl_Cs17 tdh_Cs17](Example 1).

SEQ ID NO: 23 DNA sequence of the plasmid pXMJ19[kbl_Re:H16 tdh_Re:H16](Example 1).

SEQ ID NO: 24 DNA sequence of the plasmid pXMJ19[tdh_Cx kbl_Cx](Example 1).

SEQ ID NO: 25 DNA sequence of the plasmid pXMJ19[kbl_Ch32 tdh_Ch32](Example 1).

SEQ ID NO: 26 DNA sequence of the plasmid pXMJ19[tdh_Cyb kbl_Cyb](Example 1).

SEQ ID NO: 27 DNA sequence of the plasmid pXMJ19[kbl_Ec tdh_Ec](Example 1).

SEQ ID NO: 28 Synthetic oligonucleotide: DNA sequence of the primer DargR_If (Example 3).

SEQ ID NO: 29 Synthetic oligonucleotide: DNA sequence of the primer DargR_Ir (Example 3).

SEQ ID NO: 30 Synthetic oligonucleotide: DNA sequence of the primer DargR_rf (Example 3).

SEQ ID NO: 31 Synthetic oligonucleotide: DNA sequence of the primer DargR_rr (Example 3).

SEQ ID NO: 32 DNA sequence of the plasmid pK18mobsacB_IBcg0054::Pg3-argFGH (Example 3).

SEQ ID NO: 33 Synthetic DNA fragment for the Pg3-argFGH operon (Example 3).

SEQ ID NO: 34 Synthetic oligonucleotide: DNA sequence of the primer argFGH_f (Example 3).

SEQ ID NO: 35 Synthetic oligonucleotide: DNA sequence of the primer argFGH_r (Example 3).

SEQ ID NO: 36 DNA sequence of the plasmid pK19mobsacB-ΔlysEG (Example 3).

SEQ ID NO: 37 Synthetic oligonucleotide: DNA sequence of the primer PsodcarAB-LA-F (Example 3).

SEQ ID NO: 38 Synthetic oligonucleotide: DNA sequence of the primer PsodcarAB-LA-R (Example 3).

SEQ ID NO: 39 Synthetic oligonucleotide: DNA sequence of the primer PsodcarAB-F (Example 3).

SEQ ID NO: 40 Synthetic oligonucleotide: DNA sequence of the primer PsodcarAB-R (Example 3).

SEQ ID NO: 41 Synthetic oligonucleotide: DNA sequence of the primer PsodcarAB-RA-F (Example 3).

SEQ ID NO: 42 Synthetic oligonucleotide: DNA sequence of the primer PsodcarAB-RA-R (Example 3).

SEQ ID NO: 43 DNA sequence of the Moorea producens gene with locus_tag BJP34_00300. It encodes for an L-arginine:glycine amidinotransferase (Example 3).

SEQ ID NO: 44 Amino acid sequence of the L-arginine:glycine amidinotransferase of Moorea producens (NCBI Accession Number WP_070390602) (Example 3).

SEQ ID NO: 45 DNA sequence of the plasmid pNP-6-36 (Example 3).

SEQ ID NO: 46 DNA sequence of the plasmid pLF338 (Example 3).

SEQ ID NO: 47 Synthetic oligonucleotide: DNA sequence of the primer MW_21_80_fw (Example 3).

SEQ ID NO: 48 Synthetic oligonucleotide: DNA sequence of the primer MW_21_81_rv (Example 3).

SEQ ID NO: 49 Synthetic oligonucleotide: DNA sequence of the primer MW_21_82_fw (Example 3).

SEQ ID NO: 50 Synthetic oligonucleotide: DNA sequence of the primer MW_21_83_rv (Example 3).

SEQ ID NO: 51 Synthetic oligonucleotide: DNA sequence of the primer MW_21_84_fw (Example 3).

SEQ ID NO: 52 Synthetic oligonucleotide: DNA sequence of the primer MW_21_85_rv (Example 3).

### EXPERIMENTAL SECTION

### Materials and Methods

### Chemicals

Unless explicitly stated, all chemicals were ordered from Merck, Sigma Aldrich, or Carl-Roth with analytical grade.

### Molecular Biology Techniques

All oligonucleotide primers were synthesized by Eurofins Genomics Germany GmbH (Ebersberg, Germany). Genomic DNA of C. glutamicum ATCC 13032 was isolated following manufacturer's instructions of the DNeasy Blood & Tissue Kits (Qiagen, Catalog #69504). DNA digestions were routinely performed using either High-Fidelity (HF®) restriction enzymes (NEB) or FastDigest Restriction Enzymes (FD; Thermo Fischer Scientific) following manufacturer's instructions. Polymerase chain reaction (PCR) was used to amplify desired DNA regions with specific DNA oligos. Q5® High-Fidelity 2× Master Mix (NEB Catalog #M0492) and Phusion® High-Fidelity DNA Polymerase (NEB catalog #M0530), which have proof-reading activity, were used following the manufacturer's instructions. PCR and restriction fragments were cleaned up using the QIAquick PCR Purification Kit (Qiagen, Catalog #28106) according to the manufacturer's instructions. When necessary, purification of DNA fragments from an agarose gel was performed using the QIAquick Gel Extraction Kit (Qiagen, Catalog #28706), according to the manufacturer's instructions. Agarose gel electrophoresis was done using 0.8-1.2% agarose (Roth, Catalog #3810.4) dissolved in 1×TAE buffer (Roth, Catalog #CL83.3). Gels were cast with Roti®Gelstain (Roth, Catalog #3865.1) and electrophoresis itself was carried out at 150 V for 25-40 min as needed. DNA assembly of backbone and inserts for cloning of plasmids was done using the NEBuilder® HiFi DNA Assembly Master Mix (NEB, Catalog #E2621). Following DNA assembly, the reaction was transformed into E. coli competent cells to obtain individual clones (see DNA transformation). Correct assemblies were verified for the presence of the insert via a either colony PCR or restriction analysis and sequence integrity was confirmed by DNA sequencing. Taq DNA Polymerase (Qiagen, catalog #201203) or SapphireAmp® Fast PCR Master Mix (Takara, catalog #RR350) were used to confirm the presence of a desired DNA segment from transformed cells following manufacturer's instructions. Plasmid DNA after cloning were routinely isolated using one of the following kits following manufacturer's instructions: QIAprep Spin Miniprep Kit (Qiagen, Catalog #27106), HiSpeed Plasmid Kits (Qiagen, Catalog #12643). DNA sequencing was performed by either Eurofins Genomics Germany GmbH (Ebersberg, Germany) or Sequiserve GmbH (Vaterstetten, Germany).

### Strains and Cultivation Conditions

Corynebacterium glutamicum ATCC13032 (Kinoshita S, Udaka S, Shimono M., J. Gen. Appl. Microbiol. 1957; 3(3): 193-205), the Corynebacterium glutamicum Type Strain/Wildtype, is commercially available at the American Type Culture Collection (ATCC) or at the DSMZ-German Collection of Microorganisms and Cell Cultures GmbH under the deposit no. DSM 20300.

Escherichia coli W3110 (ATCC 27325, K12 W3110) is commercially available e.g. at the DSMZ-German Collection of Microorganisms and Cell Cultures GmbH under the deposit no. DSM 5911.

Corynebacterium glutamicum ATCC 13032 and derivatives thereof were routinely grown at 30° C. in Brain heart infusion (BHI; Merck Millipore, Catalog #1104930500) broth or in CgXII minimal medium (Keilhauer et al., 1993) supplemented with 10 g/L glucose, 1 g/L L-arginine and 0.5 g/L L-threonine, as specified. The exact composition of the CgXII minimal medium without any carbon sources is detailed in Table 1. The genome sequence of C. glutamicum ATCC 13032 can be accessed under NCBI Accession Number: NC_003450. Glycerol stocks of C. glutamicum made by mixing 900 μL of an overnight culture in BHI medium (30° C., 200 rpm, 10 mL medium) with 600 μL of sterile 86% (w/v) glycerol solution and then stored at −80° C. Cultivation in 96-well deep-well plates (DWP) (Axygen, Catalog #P-2ML-SQ-C-S) for glycerol stocks was carried out by inoculating 1.8 mL of medium with a single colony. The plate was then covered using a gas-permeable membrane (M2P Labs, Catalog #F-GP-10) and incubated overnight at 30° C., 1200 rpm shaking in Heidolph incubator 1000. Glycerol stocks were made by mixing 900 μL of the overnight culture with 900 μL of 60% (v/v) glycerol and sealing the plate with aluminum sealing tape (Corning Costar, product #6569) before storing at −80° C. Escherichia coli strains were routinely grown at 37° C. in Lysogeny Broth (LB) medium (Sigma, Catalog #L3022-1 KG) or in SOB medium (Roth, Catalog #AE27.1), unless otherwise stated. Solid medium was supplemented with 1.5% (w/v) agar. Antibiotics were used for selection purposes when needed at the following concentrations: chloramphenicol—34 mg/L for E. coli and 7.5 mg/L for C. glutamicum; kanamycin—25-50 mg/L for E. coli and 15-25 mg/L for C. glutamicum. Induction of genes from pXMJ19 was done by addition of 0.1 or 0.5 mM isopropyl β-D-1-thiogalactopyranoside (IPTG) as final concentration to the medium at the start of cultivation, as stated.

TABLE 1
Composition of CgXII minimal medium without carbon sources

| Component | Final concentration in medium | |
|---|---|---|
| (NH₄)₂SO₄ | 20 | g/L |
| Urea | 5 | g/L |
| K₂HPO₄ | 1 | g/L |
| KH₂PO₄ | 1 | g/L |
| MOPS | 42 | g/L |
| MgSO₄•7 H₂O | 0.25 | g/L |
| CaCl₂•2H₂O | 13.2 | mg/L |
| FeSO₄•7 H₂O | 10 | mg/L |
| MnSO₄•H₂O | 10 | mg/L |
| ZnSO₄•7 H₂O | 1 | mg/L |
| CuSO₄ | 0.2 | mg/L |
| NiCl₂•6 H₂O | 0.02 | mg/L |
| Biotin | 0.2 | mg/L |
| Protocatechuic acid | 2.5 | g/L |

### DNA Transformation

Commercial E. coli chemical or electrocompetent cells were used for cloning and amplification of plasmid DNA (NEB, Catalog #C3019, C3020, C3040) following the manufacturer's instructions. After the recovery period, cells were spread on LB agar plates with the appropriate antibiotic for selection.

E. coli W3110 cells were made chemically competent and transformed as follows. A single colony from plate was used to inoculate 10 mL of SOB and grown overnight at 37° C., 200 rpm as a preculture. A 1:100 dilution in fresh SOB medium was inoculated as the main culture and incubated until it reached an OD₆₀₀ of 0.5-0.9. The culture was then cooled on ice for 15 min and aliquoted to falcon tubes for centrifugation (15 min, 4° C., 1100 g). The supernatant was discarded, and the cells were resuspended in 2×20 mL of ice-cold TfBI (30 mM potassium acetate, 50 mM MnCl₂, 100 mM KCl, 10 mM CaCl₂), 15% (v/v) glycerol) before centrifugation (15 min, 4° C., 1100 g). Next, the supernant as discarded and the cell pellet was resuspended a total of 5 mL of ice-cold TfB II (10 mM MOPS-KOH pH 7.0, 75 mM CaCl₂), 10 mM KCl, 15% (v/v) glycerol). The cell suspension was incubated for 20 min on ice before making 100 μL aliquots. The chemically competent cells were stored at −80° C. For transformation of E. coli W3110 competent cells, aliquots were thawed on ice for 5-15 min and then the DNA was added and mixed gently. The mixture was further incubated on ice for 10 min. Heat shock was done at 42° C. for 45 s. Cells were place on ice immediately after heat shock. The cells were recovered in 700 μL of SOB medium with incubation at 30° C. for 1 h. After the recovery period, cells were spread on appropriate selection agar plates and incubated at 30° C. to get single colonies.

C. glutamicum strains were made electrocompetent using a modified protocol: a 100 mL culture of BHI+0.5 M Sorbitol was inoculated from an overnight preculture grown at 33° C., 250 rpm to an OD₆₀₀ of 0.3 and incubated at 33° C., 130 rpm until it reached an OD₆₀₀ of 1.75. The culture was transferred to 50 mL falcon tubes and collected by centrifugation (3400 g, 10 min, 4° C.). The cell pellet was combined and washed twice with 50 mL of ice-cold Tris-glycerol buffer (1 mM Tris-HCl pH 7.5, 10% (v/v) glycerol), followed by 2 wash steps with 50 mL of ice-cold 10% (v/v) glycerol. The final cell pellet was resuspended in 800 μL of ice-cold 10% (v/v) glycerol. Electrocompetent cells were aliquoted as 150 μL and stored at −80° C. For electroporation, an aliquot of cells was thawed on ice and DNA was added to it. The cells-DNA mixture was transferred to a 2 mm electroporation cuvette (Sigma-Aldrich, Catalog #Z706088-50EA) and a 2.5 kV, 200Ω, 25 μF pulse with a 5 ms time constant was triggered using a Gene Pulser Xcell™ (Bio-Rad). The cuvette was placed on ice briefly and the contents were transferred to 4 mL of BHI (prewarmed to 46° C.) and a 6 min heat shock at 46° C. was applied to the cells. Cells were recovered at 30° C., 200 rpm for 50 min in the BHI medium. After recovery, the cells were spread on BHI agar plates with the appropriate antibiotic for selection and incubated at 30° C. for 2-3 days to get single cell colonies. Correct maintenance of replicative plasmids after transformation was verified by plasmid DNA isolation using the QIAprep Spin Miniprep Kit (Qiagen, Catalog #27106X4) following manufacturer's instructions with the exception that cells resuspended in Buffer P1 and incubated at 37° C. for 2 h before lysis. Isolated plasmid DNA was then verified by restriction analysis.

Allelic Exchange in C. glutamicum

Allele replacements, both for gene deletion and integration of genes at defined loci, were done using pK18mobsacB (NCBI Accession Number: FJ437239) (Schafer et al., 1994) derivative plasmids containing an upstream and downstream homologous region as well as the desired insert in the case of integration. The appropriate plasmid was transformed into C. glutamicum strains by electroporation as previously mentioned. After electroporation, the first recombination event, yielding an intermediate strain, was selected for on BHI agar medium containing kanamycin. Intermediate strains were screened via colony PCR for the single crossover event. Next, positive intermediate strains were counterselected on BHI+10% (w/v) sucrose agar plates without antibiotic to promote the second recombination event. After counterselection, sucrose resistant and kanamycin sensitive colonies were screened by colony PCR and DNA sequencing using appropriate primers outside of the homologous regions to confirm the desired allelic exchange took place.

Cultivation of E. coli and C. glutamicum strains for glycine production as described in Example 2 The preculture was done in 96-well DWP filled with 1.7 mL of the corresponding medium—SOB+20 mM glucose+Chloramphenicol (34 mg/L) for E. coli W3110 derivatives and BHI+Chloramphenicol (7.5 mg/L) for C. glutamicum derivatives. Each well was inoculated from a glycerol stock of the appropriate strain. The plate was then covered using a gas-permeable membrane (M2P Labs, Catalog #F-GP-10) and incubated overnight at 30° C., 1200 rpm shaking in Heidolph incubator 1000. A fresh DWP was pre-filled with 1.45 mL of production medium for E. coli W3110 derivative strains (see Table 2) and 1.5 mL of CgXII medium+2% glucose+2 g/L L-threonine+34 mg/L Chloramphenicol. The basic components of CgIII medium are detailed in Table 1, with the exception that a concentration of 30 mg/L, instead of 2.5 g/L, for protocatechuic acid was used. To the pre-filled DWP, 250 μL of the preculture for E. coli and 200 μL for C. glutamicum strains was used as inoculum resulting in an approx. start OD₆₀₀ of 0.3. After inoculation, the DWP was covered with a gas-permeable membrane and incubated at 30° C., 1200 rpm in a Heidolph incubator 1000. Gene expression was induced with 0.5 mM IPTG after 5.5 h of incubation for both E. coli and C. glutamicum strains. After 50 h, the OD₆₀₀ was measured in a spectrophotometer, the cultures were spun down (2000 g, 15 min, 20° C.), and supernatants were used to measure GAA production.

TABLE 2
Composition of glycine production
medium for E. coli W3110 strains

| | |
|---|---|
| Component | Final concentration in medium |

| | | |
|---|---|---|
| Na₂HPO₄ | 6.78 | g/L |
| KH₂PO₄ | 3 | g/L |
| NaCl | 0.5 | g/L |
| NH₄Cl | 1 | g/L |
| MgSO₄•7 H₂O | 0.25 | g/L |
| Glucose | 20 | g/L |
| HCl (37%) | 36.5 | mg/L |
| MnCl₂•7 H₂O | 1.91 | mg/L |
| ZnSO₄•7 H₂O | 1.87 | mg/L |
| Na₂ EDTA•2 H₂O (Titriplex III) | 0.84 | mg/L |
| H₃BO₃ | 0.3 | mg/L |
| Na₂MoO₄•2 H₂O | 0.25 | mg/L |
| CaCl₂•2H₂O | 4.7 | mg/L |
| FeSO₄•7 H₂O | 17.8 | mg/L |
| CuSO₄•2 H₂O | 0.15 | mg/L |
| L-Threonine | 2 | g/L |
| Chloramphenicol | 34 | mg/L |

Cultivation of C. glutamicum Strains for GAA Production as Described for Example 4

The appropriate C. glutamicum strains were used to inoculate precultures from glycerol stocks. Precultures were done in 10 mL of BHI medium, with the appropriate antibiotic as needed, in 100 mL baffled Erlenmeyer flasks and incubated at 30° C., 200 rpm for 24 h. Precultures were collected by centrifugation (3100 g, 10 min, at room temperature (RT)) and washed with 5 mL of CgXII medium without carbon sources (see Table 1). The washed pellet was resuspended in 2.5 mL of CgXII medium without carbon sources and the OD₆₀₀ of the cell suspension was determined using an Ultraspec 2100 pro (Amersham Biosciences) spectrophotometer. The appropriate volume of washed cells was used to inoculate CgXII medium supplemented with 10 g/L glucose, 1 g/L L-arginine, and 0.5 g/L L-threonine, with the appropriate antibiotic as needed, as the main culture to a start OD₆₀₀ of 0.5. Induction of genes from pXMJ19-derived plasmids was done by addition of 0.1 mM isopropyl β-D-1-thiogalactopyranoside (IPTG) as final concentration to the production medium at the start of cultivation. The main cultures were transferred to a 48-well multitier plate (Beckman Coulter Life Sciences, Catalog #M2P-MTP-48-BOH1) using 800 μL per well and the plate was covered with a sealing foil (Beckman Coulter Life Sciences/m2p Labs, Catalog #F-GPR48-10). The covered plate was placed in a BioLector® I (Beckman Coulter Life Sciences/m2p Labs) for incubation at 1400 rpm, 30° C. for 48 h. After 48 h, the OD₆₀₀ was measured in a spectrophotometer, the cultures were spun down (2000 g, 10 min), and supernatants were used to measure GAA production.

Cultivation of C. glutamicum Strains for GAA Production as Described for Example 5

The appropriate C. glutamicum strains were used to inoculate precultures from glycerol stocks. Precultures were done in 10 mL of BHI medium, with the appropriate antibiotic as needed, in 100 mL baffled Erlenmeyer flasks and incubated at 30° C., 200 rpm for 24 h. Precultures were collected by centrifugation (3100 g, 10 min, at room temperature (RT)) and washed with 5 mL of CgXII medium without carbon sources (see Table 1). The washed pellet was resuspended in 2.5 mL of CgXII medium without carbon sources and the OD₆₀₀ of the cell suspension was determined using an Ultraspec 2100 pro (Amersham Biosciences) spectrophotometer. The appropriate volume of washed cells was used to inoculate CgXII medium supplemented with 8 g/L L-threonine and 2 g/L L-arginine, with the appropriate antibiotic as needed, as the main culture to a start OD₆₀₀ of 0.5. L-threonine was added as the main source of carbon in the medium instead of glucose. Induction of genes from pXMJ19-derived plasmids was done by addition of 0.1 mM isopropyl β-D-1-thiogalactopyranoside (IPTG) as final concentration to the production medium at the start of cultivation. The main cultures were transferred to a 48-well multitier plate (Beckman Coulter Life Sciences, Catalog #M2P-MTP-48-BOH1) using 800 μL per well and the plate was covered with a sealing foil (Beckman Coulter Life Sciences/m2p Labs, Catalog #F-GPR48-10). The covered plate was placed in a BioLector® I (Beckman Coulter Life Sciences/m2p Labs) for incubation at 1400 rpm, 30° C. for 48 h. After 48 h, the OD₆₀₀ was measured in a spectrophotometer, the cultures were spun down (2000 g, 15 min, 14° C.), and supernatants were used to measure GAA production.

### Quantification of GAA

GAA from culture supernatants were quantified by HPLC-UV on a Dionex Ultimate 3000 System (Thermo Scientific). Samples were filtered with Sartorius Minisart NML Plus 0.2 μm (Sartorius AG, Catalog #ST17823-K) and then diluted appropriately in Mobile phase A (see below). Samples (10 μL injection volume) were separated using a HyperCarb 100×4.6 mm 7 μm column (Thermo Scientific catalog no. 35007-104630) at 35° C. Mobile phase A consisted of 2.3 g Ammonium dihydrogen phosphate and 2.6 g di-Ammonium hydrogen phosphate dissolved in 2 L of purified water. Mobile phase B consisted of 2.3 g Ammonium dihydrogen phosphate and 2.6 g di-Ammonium hydrogen phosphate dissolved in 1.25 L of purified water and 0.75 L acetonitrile. A flow rate of 1.0 mL min⁻¹ was maintained constant throughout the run. The column was equilibrated with 100% mobile phase A beforehand and a gradient between phases A and B was used: 0 to 8 min—phase B linear gradient increased from 0-10%; 8 to 10 min—linear gradient increased from 10-40% B; 10 to 11 min—40% B; 11.1 to 13 min—0% B; and 13 to 14 min—constant at 0% B to re-equilibrate the column. The total run duration was 14 min. Under these conditions, GAA had a retention time of 5.8 min. GAA was detected at 200 nm (210 nm as reference) using a UV Detector (ThermoScientific Dionex Ultimate 3000 DAD).

### Experimental Results

Example 1: Cloning of tdh-kbl operons for expression in C. glutamicum and E. coli Glycine is produced biosynthetically by cells in a two-step process using an L-threonine 3-dehydrogenase (EC 1.1.1.103), also called threonine dehydrogenase (Tdh), followed by a glycine C-acetyltransferase (EC 2.3.1.29), alternative name: 2-amino-3-ketobutyrate coenzyme A ligase (Kbl), which convert threonine into glycine and acetyl CoA.

The threonine dehydrogenase gene product is encoded by the tdh gene, while the glycine C-acetyltransferase gene product is encoded by the kbl gene. These genes are naturally encoded as operons in organisms. Although the gene order of the operon may vary depending on the organism source, in the present invention, we collectively refer to them as tdh-kbl operons for simplicity. We selected a total of 9 Tdh and Kbl proteins originating from Aliivibrio fischeri, Bdellovibrio sp., Bos taurus, Caulobacter sp. 17J65-9, Caulobacter sp. 17J65-9, Cupriavidus necator H16, Corynebacterium xerosis, Cyanobacteria bacterium isolate (MAG with Accession No. JAIEMW010000004.1), and Escherichia coli K12 (see Table 3 and Table 4 for full details). The proteins from B. taurus were made into a synthetic operon with the addition of ribosomal binding sequences (RBS) upstream of each gene. For the B. taurus Tdh and Kbl proteins, a shortened version without the (putative) mitochondrial signaling peptide termed Tdh(*S51M)_Bot (SEQ ID NO: 3) and Kbl(*Q21M) Bot (SEQ ID NO: 13), respectively, were used.

TABLE 3
Origin, Accession Number and SEQ ID NO of L-threonine 3-dehydrogenase (EC 1.1.1.103) proteins used in this invention

| | | | | Protein Accession | |
|---|---|---|---|---|---|
| Protein (EC number) | Gene | Organism of origin | Abbreviation | Number | SEQ ID NO: |
| L-threonine 3-dehydrogenase (EC | tdh | Aliivibrio fischeri | Tdh_Afi | WP_063649022.1 | SEQ ID NO: 1 |
| 1.1.1.103) | | | | | |
| L-threonine 3-dehydrogenase (EC | tdh | Bdellovibrio sp. | Tdh_Bds | MBI2521238.1 | SEQ ID NO: 2 |
| 1.1.1.103) | | | | | |
| L-threonine 3-dehydrogenase (EC | tdh | Bos taurus | Tdh(*S51M)_Bot | Uniprot: Q0P5L8 with an | SEQ ID NO: 3 |
| 1.1.1.103) | | | | N-terminal truncation so | |
| | | | | that S51M is the starting | |
| | | | | amino acid. | |
| L-threonine 3-dehydrogenase (EC | tdh | Caulobacter sp. 17J65-9 | Tdh_Cs17 | NEX93760.1 | SEQ ID NO: 4 |
| 1.1.1.103) | | | | | |
| L-threonine 3-dehydrogenase (EC | tdh | Cupriavidus necator H16 | Tdh_Re: H16 | QCC03509.1 | SEQ ID NO: 5 |
| 1.1.1.103) | | | | | |
| L-threonine 3-dehydrogenase (EC | tdh | Corynebacterium xerosis | Tdh_Cx | SLN02629.1 | SEQ ID NO: 6 |
| 1.1.1.103) | | | | | |
| L-threonine 3-dehydrogenase (EC | tdh | Chlamydia sp. 32-24 | Tdh_Ch32 | OJU80485.1 | SEQ ID NO: 7 |
| 1.1.1.103) | | | | | |
| L-threonine 3-dehydrogenase (EC | tdh | Cyanobacteria bacterium | Tdh_Cyb | MBY0450473.1 | SEQ ID NO: 8 |
| 1.1.1.103) | | isolate (MAG with Accession | | | |
| | | No. JAIEMW010000004.1) | | | |
| L-threonine 3-dehydrogenase (EC | tdh | Escherichia coli K12 | Tdh_Ec | P07913.1 | SEQ ID NO: 9 |
| 1.1.1.103) | | | | | |

TABLE 4
Origin, Accession Number and SEQ ID NO of glycine C-acetyltransferase (EC 2.3.1.29) (alternative
name: 2-amino-3-ketobutyrate coenzyme A ligase) proteins used in this invention

| Protein (EC number) | Gene | Organism of origin | Abbreviation | Protein Accession Number | SEQ ID NO: |
|---|---|---|---|---|---|
| glycine C-acetyltransferase | kbl | Aliivibrio fischeri | Kbl_Afi | MUJ39181.1 | SEQ ID NO: 10 |
| (EC 2.3.1.29) | | | | | |
| glycine C-acetyltransferase | kbl | Bdellovibrio sp. | Kbl_Bds | MBI2521239.1 | SEQ ID NO: 11 |
| (EC 2.3.1.29) | | | | | |
| glycine C-acetyltransferase | kbl | Bos taurus | Kbl(*Q21M)_Bot | NP_001039569.1; Uniprot - | SEQ ID NO: 12 |
| (EC 2.3.1.29) | | | | Q2KIR8 with an N-terminal | |
| | | | | truncation so that Q21M is | |
| | | | | the starting amino acid. | |
| glycine C-acetyltransferase | kbl | Caulobacter sp. 17J65-9 | Kbl_Cs17 | WP_163261703.1 | SEQ ID NO: 13 |
| (EC 2.3.1.29) | | | | | |
| glycine C-acetyltransferase | kbl | Cupriavidus necator H16 | Kbl_Re: H16 | WP_011616818.1 | SEQ ID NO: 14 |
| (EC 2.3.1.29) | | | | | |
| glycine C-acetyltransferase | kbl | Corynebacterium xerosis | Kbl_Cx | SLN02628.1 | SEQ ID NO: 15 |
| (EC 2.3.1.29) | | | | | |
| glycine C-acetyltransferase | kbl | Chlamydia sp. 32-24 | Kbl_Ch32 | OJU80486.1 | SEQ ID NO: 16 |
| (EC 2.3.1.29) | | | | | |
| glycine C-acetyltransferase | kbl | Cyanobacteria bacterium | Kbl_Cyb | MBY0450474.1 | SEQ ID NO: 17 |
| (EC 2.3.1.29) | | isolate (MAG with Accession | | | |
| | | No. JAIEMW010000004.1) | | | |
| glycine C-acetyltransferase | kbl | Escherichia coli K12 | Kbl_Ec | P0AB77.1 | SEQ ID NO: 18 |
| (EC 2.3.1.29) | | | | | |

The DNA sequence of the various tdh-kbl operons was lightly codon optimized by exchanging all rare codons towards more frequently used ones in C. glutamicum and/or E. coli. Each gene with necessary cloning overlaps was ordered as linear DNA strings from GeneArt (Thermo Fischer Scientific). Each operon was cloned into pXJM19 (Jakoby et al., 1999), a well-known E. coli-C. glutamicum shuttle vector, yielding the corresponding tdh-kbl expression plasmid. For example, the expression plasmid pXMJ19[kbl_Afi tdh_Afi] corresponds to the tdh-kbl operon from A. fischeri cloned in pXMJ19. The various tdh-kbl operons used as well as the origin of the operon, cloning strategy, and SEQ ID NO of the cloned expression plasmids are given in Table 5.

TABLE 5
Cloning and SEQ ID NO of plasmids used to express tdh-kbl operons in E. coli and C. glutamicum

| | | | SEQ ID |
|---|---|---|---|
| | | | NO of |
| Plasmid Name | Organism of origin | Cloning strategy | Plasmid |
| pXMJ19[kbl_Afi | Aliivibrio fischeri | Backbone: HindIII, SalI linearized pXMJ19. Inserts: Afkbl-string | SEQ ID |
| tdh_Afi] | | (GeneArt); Aftdh-string (GeneArt). Assembled via HiFi Assembly mix. | NO: 19 |
| pXMJ19[tdh_Bds | Bdellovibrio sp. | Backbone: HindIII, SalI linearized pXMJ19. Inserts: Bdello-tdh-string | SEQ ID |
| kbl_Bds] | | (GeneArt); Bdello-kbl-string (GeneArt). Assembled via HiFi Assembly | NO: 20 |
| | | mix. | |
| pXMJ19[kbl(*Q21M)_Bot | Bos taurus | Backbone: HindIII, SalI linearized pXMJ19. Inserts: Bt-tdh_S51M-string | SEQ ID |
| tdh(*S51M)_Bot] | | (GeneArt); Bt-kbl_Q21M-string (GeneArt). Assembled via HiFi | NO: 21 |
| | | Assembly mix. | |
| pXMJ19[kbl_Cs17 | Caulobacter sp. 17J65-9 | Backbone: HindIII, SalI linearized pXMJ19. Inserts: Caulo-tdh-string | SEQ ID |
| tdh_Cs17] | | (GeneArt); Caulo-kbl-string (GeneArt). Assembled via HiFi Assembly | NO: 22 |
| | | mix. | |
| pXMJ19[kbl_Re: H16 | Cupriavidus necator H16 | Backbone: HindIII, SalI linearized pXMJ19. Inserts: Cn-tdh-string_new | SEQ ID |
| tdh_Re: H16] | | (GeneArt); Cn-kbl-string_new (GeneArt). Assembled via HiFi Assembly | NO: 23 |
| | | mix. | |
| pXMJ19[tdh_Cx | Corynebacterium xerosis | Backbone: HindIII, SalI linearized pXMJ19. Inserts: Cxer-tdh-string | SEQ ID |
| kbl_Cx] | | (GeneArt); Cxer-kbl-string (GeneArt). Assembled via HiFi Assembly | NO: 24 |
| | | mix. | |
| pXMJ19[kbl_Ch32 | Chlamydia sp. 32-24 | Backbone: HindIII, SalI linearized pXMJ19. Inserts: Chlam-tdh-string | SEQ ID |
| tdh_Ch32] | | (GeneArt); Chlam-kbl-string (GeneArt). Assembled via HiFi Assembly | NO: 25 |
| | | mix. | |
| pXMJ19[tdh_Cyb | Cyanobacteria bacterium | Backbone: HindIII, SalI linearized pXMJ19. Inserts: Cyano-tdh3-string | SEQ ID |
| kbl_Cyb] | isolate (MAG | (GeneArt); Cyano-kbl-string (GeneArt). Assembled via HiFi Assembly | NO: 26 |
| | with Accession | mix. | |
| | No. JAIEMW010000004.1) | | |
| pXMJ19[kbl_Ec | Escherichia coli K12 | Backbone: HindIII, SalI linearized pXMJ19. Inserts: Ec-tdh3-string | SEQ ID |
| tdh_Ec] | | (GeneArt); Ec-kbl-string (GeneArt). Assembled via HiFi Assembly mix. | NO: 27 |

Example 2: Impact of implementation of Tdh-Kbl activities on glycine production from L-threonine A wildtype strain of E. coli was used as a platform for glycine production from L-threonine by overexpression of tdh-kbl operons leading to enhanced L-threonine 3-dehydrogenase (EC 1.1.1.103) and glycine C-acetyltransferase (EC 2.3.1.29) activity. E. coli W3110 (available as DSM 5911 and ATCC 27325) was transformed with the plasmids listed in Table 5 as well as the empty vector, pXJM19, as a reference. Subsequently, these strains were tested for glycine production in minimal medium supplemented with L-threonine as described in the Materials and Methods section. The introduction of Tdh and Kbl proteins was enough to generate an increased amount of glycine compared to the starting strain as shown in Table 6.

TABLE 6
Glycine production of E. coli W3110
with enhanced Tdh and Kbl activities

| Strain | Glycine (mg/L) |
|---|---|
| W3110 + pXMJ19 | n.d. |
| W3110 + pXMJ19[kbl_Afi tdh_Afi] | 80 |
| W3110 + pXMJ19[tdh_Bds kbl_Bds] | 20 |
| W3110 + pXMJ19[kbl(*Q21M)_Bot tdh(*S51M)_Bot] | 25 |
| W3110 + pXMJ19[kbl_Cs17 tdh_Cs17] | 50 |
| W3110 + pXMJ19[kbl_Re: H16 tdh_Re:H16] | 50 |
| W3110 + pXMJ19[tdh_Cx kbl_Cx] | 10 |
| W3110 + pXMJ19[kbl_Ch32 tdh_Ch32] | 20 |
| W3110 + pXMJ19[tdh_Cyb kbl_Cyb] | 45 |
| W3110 + pXMJ19[kbl_Ec tdh_Ec] | 110 |

n.d.—not detected.

Table 6 shows that E. coli W3110 strains with the various Tdh and Kbl proteins produced between 10 and 110 mg/L of glycine from L-threonine while no glycine was detected for the reference strain W3110+pXJM19. Thus, we conclude that the introduction or enhancement of Tdh and Kbl activity improves glycine production in microorganisms, such as E. coli.

### Example 3: Construction of LF-S-677a for Chromosomal Expression of an AGAT and Increased L-Arginine Availability in C. glutamicum

3.1 Chromosomal Deletion of the argR Gene in C. glutamicum ATCC 13032 Derivative Strains

To improve intracellular L-Arginine formation and L-Arginine recycling from L-Ornithine, the argR gene coding for the central repressor protein ArgR controlling the L-arginine biosynthetic pathway was inactivated. Inactivation was achieved via allele replacement with the plasmid pK18mobsacB_DargR.

pK18mobsacB_DargR was Cloned as Follows:

Backbone: XbaI linearized pK18mobsacB. Insert(s): PCR of ATCC 13032 genomic DNA with oligos DargR_If (SEQ ID NO: 28) and DargR_Ir (SEQ ID NO: 29), PCR of ATCC 13032 genomic DNA with oligos DargR_rf (SEQ ID NO: 30) and DargR_rr (SEQ ID NO: 31). Assembled via NEBuilder HiFi DNA Assembly.

3.2 Chromosomal Expression of the Arginine Biosynthesis Genes argF, argG, argH Under the Control of the Strong Constitutive Promoter Pg3 in C. glutamicum ATCC 13032 Derivative Strains

To enhance the activities of ArgF, ArgG, and ArgH, additional copies of the corresponding genes were inserted into the genome. This was achieved via allele replacement with the plasmid pK18mobsacB_IBcg0054::Pg3-argFGH (SEQ ID NO: 32).

pK18mobsacB_IBcg0054::Pg3-argFGH (SEQ ID NO: 32) was Cloned as Follows:

First, a synthetic operon consisting of Pg3, argF, argG, argH and flanking regions for genomic integration (SEQ ID NO: 33) was designed. The DNA sequence was ordered for gene synthesis from Invitrogen/GeneArt (Thermo Fisher Scientific, Waltham, USA) and it was delivered as part of a cloning plasmid with an ampicillin resistance gene (designated as pMA-RQ_argFGH). Backbone: EcoRI, HindIII linearized pK18mobsacB. Insert(s): PCR of PCR with pMA-RQ_argFGH with oligos argFGH_f (SEQ ID NO: 34) and argFGH_r (SEQ ID NO: 35). Assembled via NEBuilder HiFi DNA Assembly.

3.3 Chromosomal Deletion of the Genes lysE and lysG in C. glutamicum ATCC 13032 Derivative Strains

The gene lysE codes for an exporter protein that catalyzes the efflux of L-Lysine, L-arginine and L-citrulline in Corynebacterium glutamicum. The expression of lysE is positively regulated by the gene product of lysG. Both genes are located next to each other but are transcribed divergently. In order to inactivate the transporter protein LysE and the positive regulator protein LysG, the plasmid pK19mobsacB-ΔlysEG (SEQ ID NO: 36) was constructed as described for pK18mobsacB-ΔlysEG (Vrljic et al., 1996). Deletion of lysEG was achieved via allele replacement using the reconstructed pK19mobsacB-ΔlysEG.

3.4 Chromosomal Insertion of the Sod Promoter Upstream of the carAB Operon in C. glutamicum ATCC 13032 Derivative Strains

To improve the production of L-arginine, the strong sod-promoter was inserted upstream of the carAB operon in the genome. This was achieved via allele replacement with the plasmid pK18mobsacB_PsodcarAB.

pK18mobsacB_PsodcarAB was Cloned as Follows:

Backbone: EcoRI, HindIII linearized pK18mobsacB. Insert(s): PCR of ATCC 13032 genomic DNA with oligos PsodcarAB-LA-F (SEQ ID NO: 37) and PsodcarAB-LA-R (SEQ ID NO: 38), PCR of ATCC 13032 genomic DNA with oligos PsodcarAB-F (SEQ ID NO: 39) and PsodcarAB-R (SEQ ID NO: 40), PCR of ATCC 13032 genomic DNA with oligos PsodcarAB-RA-F (SEQ ID NO: 41) and PsodcarAB-RA-R (SEQ ID NO: 42). Assembled via NEBuilder HiFi DNA Assembly.

3.5 Chromosomal Insertion of the Gene AGAT-Mp Coding for an L-Arginine:Glycine Amidinotransferase (AGAT; EC 2.1.4.1) from Moorea producens

A heterologous gene encoding for an L-arginine:glycine amidinotransferase (AGAT), EC 2.1.4.1, is required for the formation of guanidinoacetic acid (GAA) from of L-arginine and glycine. Moorea producens is a filamentous cyanobacterium. The genome of the M. producens strain PAL-8-15-08-1 can be accessed under Genbank accession Number CP017599.1 (Leao et al., 2017). It contains an open reading frame coding for a L-arginine:glycine amidinotransferase (AGAT, EC 2.1.4.1; locus_tag BJP34_00300 shown in SEQ ID NO: 43). SEQ ID NO: 44 shows the corresponding amino acid sequence (NCBI Accession Number WP_070390602). The AGAT-Mp expression cassette was assembled into a plasmid as an intermediate step to facilitate future cloning yielding pNP-6-36 (SEQ ID NO: 45). pNP-6-36 contains a promoter sequence, ribosomal binding site, and a codon-optimized version of the AGAT-Mp gene. The corresponding gene product has the same amino acid sequence given in SEQ ID NO: 44. The AGAT-Mp expression cassette was integrated at the intergenic region of NCg10013_NCg10014 into the genome of C. glutamicum via allele replacement using pLF338 (SEQ ID NO: 46). pLF338 is a pK18mobsacB derivative containing the homologous regions needed for integration at the intergenic region of NCg10013_NCg10014 as well as the expression cassette for the AGAT-Mp gene.

The cloning of pLF388 was done in multiple parts. First, pLF337 was constructed as a pK18mobsacB derivative containing the homologous regions for integration into the genome. pLF337 was cloned as follows:

Backbone: EcoRI linearized pK18mobsacB. Insert(s): PCR of ATCC 13032 genomic DNA with oligos MW_21_80_fw (SEQ ID NO: 47) and MW_21_81_rv (SEQ ID NO: 48), PCR of ATCC 13032 genomic DNA with oligos MW 21_82_fw (SEQ ID NO: 49) and MW_21_83_rv (SEQ ID NO: 50).

Assembled via NEBuilder HiFi DNA Assembly.

Finally, the AGAT-Mp expression cassette from pNP-6-36 was cloned into pLF337 yielding pLF338 as the plasmid for chromosomal integration of the AGAT in C. glutamicum.

pLF338 was Cloned as Follows:

Backbone: AscI linearized pLF337. Insert(s): PCR of pNP-6-36 with oligos MW_21_84_fw (SEQ ID NO: 51) and MW_21_85_rv (SEQ ID NO: 52). Assembled via NEBuilder HiFi DNA Assembly.

### Example 4: Impact of Implementation of Tdh-Kbl Activities on GAA Production from Glucose, L-Threonine, and L-Arginine

The C. glutamicum strain LF-S-677a (genotype: ATCC 13032 ΔargR IBcg0054::Pg3-argFGH ΔlysEG Psod-carAB intNCgl0013-NCgl0014::{P2947MU}[agat_Mpr]), whose construction was described in Example 3, served as our basis strain. This strain has an increased ability to provide L-arginine (through deletion or attenuation of ArgR and LysEG, enhancement of ArgF, ArgG, and ArgH and of CarAB) and was equipped with a heterologous L-arginine:glycine amidinotransferase (AGAT-Mp). This basis strain was further modified by overexpression of the tdh-kbl_Afi operon with pXMJ19[kbl_Afi tdh_Afi](SEQ ID NO: 19); resulting in enhanced L-threonine 3-dehydrogenase (EC 1.1.1.103) and glycine C-acetyltransferase (EC 2.3.1.29) activities. We tested these strains for GAA production in a production minimal medium supplemented with 10 g/L glucose, 1 g/L L-arginine, and 0.5 g/L L-threonine (see full description in Materials and Methods section). Surprisingly, the introduction of Tdh and Kbl proteins was enough to generate an increased amount of GAA production compared to the starting strain as shown in Table 7.

TABLE 7
GAA production of C. glutamicum LF-S-677a strains with enhanced
Tdh and Kbl activities from glucose, L threonine, and L-arginine

| | GAA produced | | |
|---|---|---|---|
| Strain | (mg/L) | OD₆₀₀ | Yield GAA/OD₆₀₀ (a.u.) |
| LF-S-677a | 89 | 19.2 | 4.6 |
| LF-S-677a + | 128 | 19.2 | 6.7 |
| pXMJ19[kbl_Afi | | | |
| tdh_Afi] | | | |

Table 7 shows that C. glutamicum LF-S-677a+pXMJ19[kbl_Afi tdh_Afi], which has enhanced Tdh and Kbl activities, can produce 128 mg/L of GAA compared to only 89 mg/L of GAA in the reference strain, LF-S-677a, from glucose, L-threonine, and L-arginine. The Yield of GAA/OD₆₀₀ was similarly higher in the strain with Tdh and Kbl compared to the parent (6.7 versus 4.6, respectively). Thus, we conclude that the introduction of Tdh and Kbl activity improves GAA production in a strain already modified to have a combination of AGAT activity, increased ability to provide L-arginine, and reduced L-arginine export.

### Example 5: Impact of Implementation of Tdh-Kbl Activities on GAA Production from L-Threonine and L-Arginine

The C. glutamicum strain LF-S-677a (genotype: ATCC 13032 ΔargR IBcg0054::Pg3-argFGH ΔlysEG Psod-carAB intNCgl0013-NCg10014::{P2947MU}[agat_Mpr]), whose construction was described in Example 3, served as our basis strain. This strain has an increased ability to provide L-arginine (through deletion or attenuation of ArgR and LysEG, enhancement of ArgF, ArgG, and ArgH and of CarAB) and was equipped with a heterologous L-arginine:glycine amidinotransferase (AGAT-Mp). This basis strain was further modified by overexpression of the tdh-kbl_Afi operon with pXMJ19[kbl_Afi tdh_Afi](SEQ ID NO: 19); resulting in enhanced L-threonine 3-dehydrogenase (EC 1.1.1.103) and glycine C-acetyltransferase (EC 2.3.1.29) activities. We tested these strains for GAA production in a production minimal medium supplemented with 8 g/L L-threonine and 2 g/L L-arginine (see full description in Materials and Methods section). The introduction of Tdh and Kbl proteins was enough to generate an increased amount of GAA compared to the starting strain as shown in Table 8.

TABLE 8
GAA production of C. glutamicum LF-S-677a strains with enhanced
Tdh and Kbl activities from L-threonine and L-arginine

| | GAA produced | | Yield GAA/OD₆₀₀ |
|---|---|---|---|
| Strain | (mg/L) | OD₆₀₀ | (a.u.) |
| LF-S-677a | 19 | 3.0 | 6.3 |
| LF-S-677a + | 68 | 3.2 | 21.3 |
| pXMJ19[kbl_Afi | | | |
| tdh_Afi] | | | |

Table 8 shows that C. glutamicum LF-S-677a+pXMJ19[kbl_Afi tdh_Afi], which has enhanced Tdh and Kbl activities, can produce 68 mg/L of GAA compared to only 19 mg/L of GAA in the reference strain, LF-S-677a, from L-threonine and L-arginine. The Yield of GAA/OD₆₀₀ was similarly higher in the strain with Tdh and Kbl compared to the parent (21.3 versus 6.3, respectively). Thus, we conclude that the introduction of Tdh and Kbl activity improves GAA production in a strain already modified to have an AGAT activity.

### CITATIONS

Jakoby, M., Ngouoto-Nkili, C.-E., & Burkovski, A. (1999). Construction and application of new Corynebacterium glutamicum vectors. Biotechnology Techniques, 13(6), 437-441. https://doi.org/10.1023/A:1008968419217
Keilhauer, C., Eggeling, L., & Sahm, H. (1993). Isoleucine synthesis in Corynebacterium glutamicum: molecular analysis of the ilvB-ilvN-ilvC operon. J Bacteriol, 175(17), 5595-5603. https://doi.org/10.1128/jb.175.17.5595-5603.1993
Leao, T., Castelao, G., Korobeynikov, A., Monroe, E. A., Podell, S., Glukhov, E., Allen, E. E., Gerwick, W. H., & Gerwick, L. (2017). Comparative genomics uncovers the prolific and distinctive metabolic potential of the cyanobacterial genus Moorea. Proc Natl Acad Sci USA, 114(12), 3198-3203. https://doi.org/10.1073/pnas.1618556114
Liu, J. Q., Nagata, S., Dairi, T., Misono, H., Shimizu, S., & Yamada, H. (1997). The GLY1 gene of Saccharomyces cerevisiae encodes a low-specific L-threonine aldolase that catalyzes cleavage of L-allo-threonine and L-threonine to glycine-expression of the gene in Escherichia coli and purification and characterization of the enzyme. Eur J Biochem, 245(2), 289-293. https://doi.org/10.1111/j.1432-1033.1997.00289.x
Schafer, A., Tauch, A., Jager, W., Kalinowski, J., Thierbach, G., & Puhler, A. (1994). Small mobilizable multi-purpose cloning vectors derived from the Escherichia coli plasmids pK18 and pK19: selection of defined deletions in the chromosome of Corynebacterium glutamicum. Gene, 145(1), 69-73. https://doi.org/10.1016/0378-1119(94)90324-7
Vrljic, M., Sahm, H., & Eggeling, L. (1996). A new type of transporter with a new type of cellular function: L-lysine export from Corynebacterium glutamicum. Mol Microbiol, 22(5), 815-826. https://doi.org/10.1046/j.1365-2958.1996.01527.x

1. A microorganism having an increased ability to produce L-arginine from L-ornithine compared with the ability of a wildtype microorganism or of a non-modified microorganism, the microorganism comprising:
at least one heterologous gene coding for a protein having the function of a L-arginine:glycine amidinotransferase,
at least one heterologous gene encoding a protein having L-threonine 3-dehydrogenase activity, and
at least one heterologous gene encoding a protein having a glycine C-acetyltransferase activity.

2. The microorganism of claim 1, wherein the increased ability to produce L-arginine from L-ornithine compared with the ability of the wildtype microorganism or of the non-modified microorganism, is effected by an inactivated or deleted gene coding for an arginine responsive repressor protein ArgR.

3. The microorganism of claim 1, wherein the increased ability to produce L-arginine from L-omithine compared with the ability of the wildtype microorganism or of the non-modified microorganism, is effected by an overexpressed gene coding for an enzyme having the function of a carbamoylphosphate synthase.

4. The microorganism of claim 1, wherein the increased ability to produce L-arginine from L-omithine compared with the ability of the wildtype microorganism or of the non-modified microorganism, is effected by at least one or more overexpressed genes selected from the group consisting of a gene coding for a protein having the function of an omithine carbamoyltransferase, a gene coding for a protein having the function of an argininosuccinate synthetase, and a gene coding for a protein having the function of an argininosuccinate lyase.

5. The microorganism of claim 1, wherein a gene coding for a protein having the function of an arginine exporter is inactivated or deleted.

6. The microorganism of claim 1, wherein the at least one heterologous gene encoding the protein having L-threonine 3-dehydrogenase activity is overexpressed.

7. The microorganism of claim 1, wherein the at least one heterologous gene encoding the protein having a glycine C-acetyltransferase activity is overexpressed.

8. The microorganism of claim 1, wherein the microorganism belongs to at least one of the genuses selected from the group consisting of: Corynebacterium, Enterobacteriaceae, and Pseudomonas.

9. The microorganism of claim 8, wherein the microorganism is Corynebacterium glutamicum.

10. A method for the fermentative production of guanidino acetic acid (GAA), the method comprising:
cultivating the microorganism as defined in claim 1 in a medium, and
accumulating GAA in the medium to form a GAA containing fermentation broth.

11. The method of claim 10, further comprising:
isolating the GAA from the GAA containing fermentation broth.

12. The microorganism of claim 1, further comprising:
a gene coding for an enzyme having the activity of a guanidinoacetate N-methyltransferase.

13. The microorganism of claim 12, wherein the gene coding for the enzyme having the activity of the guanidinoacetate N-methyltransferase is overexpressed.

14. A method for the fermentative production of creatine, the method comprising:
cultivating the microorganism of claim 1 in a medium, and
accumulating creatine in the medium to form a creatine containing fermentation broth.

15. The method of claim 14, further comprising: isolating the creatine from the creatine containing fermentation broth.


SEQUENCE LISTING (52 sequences)

SEQ ID NO: 1 - protein, 343 aa, Aliivibrio fischeri
MKIKALSKLKPEEGIWLTEVEKPEMGHNDLLIRIKKTAICGTDVHIYNWDEWSQKTIPVPMVVGHEYVGEVVGIGQEVRGFEIGDRVSGEGHITCGHCRNCRGGRTHLCRNTTGVGVNRTGAFSEYLVIPAFNAFKIPAGISDDLASIFDPFGNAVHTALSFDLVGEDVLITGAGPIGIMAAAVAKHVGARHVVITDVNEYRLDLAKKMGVTRAVNVMNEKLEDVMSDLGMTEGFDVGLEMSGNPSAFNSMLTNMNHGGKISLLGIPPSDMTVDWNQVIFKGLVIKGIYGREMFETWYKMASLIQSGLDLTPIITHHYKIDDFQAGFDMMRSGMSGKVILDWE

SEQ ID NO: 2 - protein, 341 aa, Bdellovibrio sp.
MKALVKKYNQPGLWMEKVDIPSIGIDDVLIKIHKTAICGTDIHIYNWDEWAQKTIPYPMVVGHEYVGTIAAIGSNVKGFEIGEVVSGEGHIVCGHCRNCRAGRRHLCMNTRGVGVNRQGSFAEFLSIPASNVWRCDPNIPQDVLACFDPFGNAVHTTLSYDLVGEDVLITGAGPIGIMAVAIARHAGARHVVITDVNPYRLGLAKKMGATRAVNVQEEKLSNVIKDLKMTEGFDIGLEMSGNVQAFKDMIDHMVHGGKIALLGILPNGTGIDWDKVVFNSLNIKGIYGREMFETWYQMTVLLQSGLDISPVITHRFPIDDFEKGFELMKSGQSGKVILNWI

SEQ ID NO: 3 - protein, 323 aa, Bos taurus
MEADQQRVLITGGLGQLGVGLASFLRKRFGKDNVILSDIRKPPEHVFLSGPFIYSDILDYKNLREIVVNNRITWLFHYSALLSAVGEANVSLARAVNITGLHNVLDVAAEHGLRLFVPSTIGAFGPTSPRNPTPDLCIQRPRTIYGVSKVHAELMGEYYYYRYGLDFRCLRYPGIISADSQPGGGTTDYAVQIFHEAVKNGRFECNLKPDTRLPMMYIDDCLRATLEVMEAPAESLSMRTYNISAMSFTPEELAQEVLKHVPELQVTYNVDPVRQAIADSWPMNFDDSNARKDWGWKHDFDLPELVTTMLNFHGSESRVAQAN

SEQ ID NO: 4 - protein, 349 aa, Caulobacter sp.
MTSTMKALAKMKPAEGLELIEAPIPTPGPEDVLIRVKRTAICGTDIHIWNWDEWSQKNVPVPMITGHEFSGEIVEIGKDVDRPLKIGQRVSAEGHVIDLNSFAARAGHFHLDPNTRGIGVNRQGAFAEYVVAPAFNVIPLPDDVSFEVASMLDPFGNAVHTAQQFDLVGEDVLVTGAGPIGIMAAAVARRAGARTVVLTDVNEYRLDLARKVADVRTVNVAKEDLKDVMKELGIQVGFDVALEMSGAPSAFNQCVDNLIMGGGLALLGIPSRPMETDWGKIILKALTIKGVYGREMFDTWRKMLGLLKAGVDLTPLITHRLAYDRYVEGFEAMKSGRSGKVVLDWDKAA

SEQ ID NO: 5 - protein, 318 aa, Cupriavidus necator
MEAGKPKILIVGANGQIGSELALALAERYGRTNVITSDVVPTGRHVHLTHEMLNATDRGELATVVERHGITQVYLLAAALSATGEKAPQWAWNLNMTSLLNVLELARQTGLERVFWPSSIAAFGPTTPAGQTPQKTVMEPTTVYGISKQAGEGWCRWYHANHGVDVRSVRYPGLISHKTPPGGGTTDYAVDIFHAAVTGEPYTCFLKEDEALPMMYMPDAIRATIELMEAPADKLSERGSYNIAGMSFTPAQIAAAIREQVPGFQIRYEPDYRQAIAQGWPDSIDDSVARADWGWKAQYGLKEMVADMLANLKATLAG

SEQ ID NO: 6 - protein, 355 aa, Corynebacterium xerosis
MMRALRKTEPGPGLTLTEVPEPACGPWDVKIRVLRAGICGTDLHILGWDDSAEAMCDTVPFTPGHEFYGEVVEAGSEVPDARIGDRVSGEGHVVCGVCRNCRAGRKQMCIRTRSVGVQRDGAFAEYVVIPHLNVWVHEHDSRTEVITPELGALFDPFGNAVHTALKFPLVGEDVLITGAGPIGLMAAAVARHAGARYVAITDVHEHRLEMARAGGVDLALNVATTTMTQAQRELGMKEGFDVGLEISGQASALQEMISNMNHGGRIALLGLPARHFELDWTTVITHMLTLQGIYGREMYETWNAMSAMISTSPLLRERVLATVSDVVPASQWESAYEIARSGRGGKVLLDWETIG

SEQ ID NO: 7 - protein, 340 aa, Chlamydia sp.
MKALVKKYSKPGLWMDEVPVPEVGDDEVLIKVKKTSICGTDVHIYKWDSWAQKTIPVPMVVGHEFMGTIEALGKNVKHLQVGQRVSGEGHITCGQCPPCKMGNKHLCMNTLGVGVNRHGCFAEYFNLPAENVFLLPDSVKDNVASIFDPYGNAVHTTLSFEVTGEDVLITGAGPIGIMAAAIAQKAGARHVVITDVNEYRLDLAKKMGATTTVNVAKQDIKEVMKELGITHGFTVGLEMSGHPNGLSTLLETAQHGAKIALLGILPPHTTVDWDLVIFKMLMVKGIYGREVFRTWYKMTNLLESGLNLEPIITHHFPIDDYEKGFEVMLSGKSGKVILDW

SEQ ID NO: 8 - protein, 359 aa, Cyanobacteria bacterium
MPAPSSTLAKTLPQTMQAIQKPAAASGFALIEAERPEPGPDDVLIQVSLASICGTDLHITQWDEWSASRIKPPLIYGHEFCGVVAAIGRNVDNVKVGDFVSAEMHLPCRTCRQCMTGNFHICEKVEIVGIDRNGCFANYVVIPRWQIIKLPASITPEVGACLDSLGNAVHAVSKGNVSGKTVYVVGCGPIGLFSIRVSKALGATRVFASDISPYRLEMAKTAGATHVLDARECNPSDWMLEATQGRGVDVVLEMSGNERAINDAFQGLALGGTMVLLGIPQGNVSLNLNRDVIFKEATVLGVNGREMFQTWFLMLELLESGRLNIDFIMTHRFALSEFETAIELIASGKSGKILLEPSF

SEQ ID NO: 9 - protein, 341 aa, Escherichia coli
MKALSKLKAEEGIWMTDVPVPELGHNDLLIKIRKTAICGTDVHIYNWDEWSQKTIPVPMVVGHEYVGEVVGIGQEVKGFKIGDRVSGEGHITCGHCRNCRGGRTHLCRNTIGVGVNRPGCFAEYLVIPAFNAFKIPDNISDDLAAIFDPFGNAVHTALSFDLVGEDVLVSGAGPIGIMAAAVAKHVGARNVVITDVNEYRLELARKMGITRAVNVAKENLNDVMAELGMTEGFDVGLEMSGAPPAFRTMLDTMNHGGRIAMLGIPPSDMSIDWTKVIFKGLFIKGIYGREMFETWYKMAALIQSGLDLSPIITHRFSIDDFQKGFDAMRSGQSGKVILSWD

SEQ ID NO: 10 - protein, 398 aa, Aliivibrio fischeri
MSSAFYTQIQNQIEEVKNEGLYKSERIITSAQKASVSISTGQEVLNFCANNYLGLANHPALIEAAKDGMDEHGFGMASVRFICGTQDSHKELEQKLSTFLGKEDTILYTSCFDANAGLFETILGAEDAIISDALNHASIIDGVRLCKAMRFRYANNNMTELEEQLIAAKEAGARHTLIVTDGVFSMDGVVANLPAICDLADKYDALVMVDDSHAVGFMGENGTGTHEYHDVIDRIDIITGTLGKAMGGASGGYTSGKKEVIDWLRQRSRPYLFSNSVAPAIVSASIRVLDLLAESGDLRSKLWENSAHFRTRMEAAGFTMGGADHAIIPIMLGDAKIAVEFAERALEKGIYVIAFSFPVVPKGQARIRTQMSAAHTREQLDRAIDAFIEVGKDMEIIK

SEQ ID NO: 11 - protein, 398 aa, Bdellovibrio sp.
MFNQMKDYFIKEMAEIKEQGLYKSERLINTPQSSHIGVVGGQEVINLCANNYLGLANHPDVLKAAKASYDKWGYGLSSVRFICGTQEIHKTLERKISEFLGTEDTILYSSCFDANGGLFETILGEEDAIISDELNHASIIDGIRLCKAKRFKYKNNDPNDLESKLKEADQSGARFKMVASDGVFSMDGYIANLKAVCELAQKYQCLVMVDDSHAVGFIGQNGRGTHEHCKVMGQVDIITGTLGKALGGASGGYTSGRKEIIEKLRQRSRPYLFSNTLAPSIVCASLKVLELLSQSDELRTKLRQNTQFFRTEMTKAGFNILKGEHPIVPVMLGDAILAQKIAARLLELGVYVVGFYFPVVPKGQARIRTQVSAAHSRTDLEFAIAQFIQVKREFSAGV

SEQ ID NO: 12 - protein, 399 aa, Bos taurus
MSALAQLRGILEEELESIRGAGTWKSERVITSRQGPHIHVDGAPGGIINFCANNYLGLSSHPEVIQAGLRTLKEFGAGLSSVRFICGTQSIHKDLEAKIARFHQREDAILYPSCFDANTGLFEALLTSEDAVLSDELNHASIIDGIRLCKAHKYRYRHLDMADLEAKLQEAQKHRLRLVATDGAFSMDGDIAPLQEICRLASQYGALVFVDESHATGFLGATGRGTDELLGVMDQVTIINSTLGKALGGASGGYTTGPGALVSLLRQRARPYLFSNSLPPAAVGCASKALDLLMESNAIVQSMAAKTLRFRSQMEAAGFTISGANHPICPVMLGDARLALNIADDMLKRGIFVIGFSYPVVPKGKARIRVQISAVHSEEDIDRCVEAFVEVGRLHGALP

SEQ ID NO: 13 - protein, 404 aa, Caulobacter sp.
MREDFYGRVRSELADIDAQGLTKPERVITSRQGADIRVKLPGGGEREVLNFCANNYLGLAGDERVTCAAIEASERWGAGTASVRFICGTMDIHKQLERAIADYLGYEDSILFAAAFDANGGVFEPLLGEEDAIVSDSLNHASIIDGVRLCKAKRYRFANSDMADLEAKLAEAKAAGARQIMIATDGAFSMDGYIAKLDQLCALADQYDALVMVDDCHATGFLGPQGRGSGAWNGVEKRVDIVTGTFGKALGGAMGGFICARKEVVDLLRQRARPYLFSNALSPAVCGASLEAIRIAAGSEGDALRKKLFDNAARFRAGMAAAGFKLLEGEHPIIPVMLGDAKLAQDMAAKLLEEGIYVIGFSFPVVPRGAARIRTQMSAAHEPHHVDAAIAAFTKVGRELGVIS

SEQ ID NO: 14 - protein, 399 aa, Cupriavidus necator
MSNAEAFYASIRTELESIRAAGLFKNERVIATPQGARVRTTDGREVINLCANNYLGLSSHPQVIEAAHEALRTHGFGLSSVRFICGTQDLHKTLEARLSAFLGTEDTILYGSAFDANGGLFETLLGAEDAVISDALNHASIIDGVRLSKARRYRYQHNDMDDLRVQLEQARADGARYTLVFSDGVFSMDGTVARLDEMRAICDEYGALLGIDECHATGFMGQRGRGTHEARGVFGKIDIITGTLGKALGGASGGFTSARKEVVALLRQRSRPYLFSNTVAPAIVGASIAVLDILEASTELRDRLEGNTRFFRAGLDRLGFDVKAGDHPIIPIMVYDADKAQQLAQRLLELGVYVVGFFYPVVPKGQARIRVQMSALHDEAALQAALDAFGQAGRELGLI

SEQ ID NO: 15 - protein, 325 aa, Corynebacterium xerosis
MASVRFICGTQDLHLELERAVSTFLGTEETILFSSCFDANGAVFEPLFGTQDAIISDALNHASLIDGIRLSKAARYRYRNADLEDLRTRLDATRDQNDGAGARRTIIVTDGVFSMDGYLAPLDGICDLADEYGALVLVDDSHAVGFMGATGAGTPEHFGVSNRVDLYTGTFGKALGGASGGYVSGRREIVAMLRQKGRPYLFSNSLAPSIVAATLAALDLVAGSAELRATLFRNAELFRRRMSEEGFDLLDGEHAIVPVMFGDAALAGRIADAMLDHGVFVTAFSYPVVPQGKARIRVQLSAAHTEADVEAAVQAFIASRAAVQG

SEQ ID NO: 16 - protein, 395 aa, Chlamydia sp.
MKESFYTHINQELENIKNLGLYKKERIITSPQQSEIAISNGQHVLNFCANNYLGLANNQQLKEAAKESLDKYGFGLSSVRFICGTQTIHKELEDKLSSFLGMEDTILYSSCWDANGGLFEILLGPEDAVISDSLNHASIIDGIRLCKAKRLRYENNNMKDLEEKLKEADGTRFKLVATDGVFSMDGTIANLQGICDLADKYNALVMVDDSHAVGFIGKKGRGTHEFCNVMGRVDIITGTLGKALGGASGGYTSAKKEVIDLLRQRSRPYLFSNTLAPSIAGASLQALKLIESSDDLRQKLIENSEYFREKMTTLGFTLPPGEHPIIPVMLGDAKLAQEFAAKMLEEGIYVIGFFYPVVPQGKARIRTQMSAVHSKQDLDKAIAAFEKVGKSLQVI

SEQ ID NO: 17 - protein, 407 aa, Cyanobacteria bacterium
MTSTNQAETPQALKSHRLDFLEAECQELKEQHLFQSLRVMSSAQGAKIQLDGKSVINLSSNNYLGLNTHPKLITAAKKAIDDYGVGTGSVRTIIGTMDIHQAFEKRLAQFKHTEATLVLQSGFTANNAAVTSILGEGDVVISDSLNHASIIDACRLLKKVPRYVYAHADMTELEARLKETQSARRRLVVTDGVFSMDGDIAPLPEIVALCEQYNAIVMVDDAHASGVFGKNGRGTVDHFNLHGRVDIQIGTLSKAIGALGGYVACTQAMRDIMINRARPFLFSTSAPPSVVMTCMAALDLLETDTALIDQLWYNTRYFKAGLKSIGYNVQSDSPIIPVIAGTSEKAHRLSERLFEEGVYVRSIVYPTVALDKARVRVILSATHTQTQLDQAIAAFEKVGKELDLIGP

SEQ ID NO: 18 - protein, 398 aa, Escherichia coli
MRGEFYQQLTNDLETARAEGLFKEERIITSAQQADITVADGSHVINFCANNYLGLANHPDLIAAAKAGMDSHGFGMASVRFICGTQDSHKELEQKLAAFLGMEDAILYSSCFDANGGLFETLLGAEDAIISDALNHASIIDGVRLCKAKRYRYANNDMQELEARLKEAREAGARHVLIATDGVFSMDGVIANLKGVCDLADKYDALVMVDDSHAVGFVGENGRGSHEYCDVMGRVDIITGTLGKALGGASGGYTAARKEVVEWLRQRSRPYLFSNSLAPAIVAASIKVLEMVEAGSELRDRLWANARQFREQMSAAGFTLAGADHAIIPVMLGDAVVAQKFARELQKEGIYVTGFFYPVVPKGQARIRTQMSAAHTPEQITRAVEAFTRIGKQLGVIA

SEQ ID NO: 19 - DNA, 8843 bp, synthetic construct
  source 1..8843: mol_type = other DNA
gagctgttgacaattaatcatcggctcgtataatgtgtggaattgtgagcggataacaatttcacacaggaaacagaattaattaagcttaacataactaaaaaaaggaaccgcaatgtcttctgcattctacacacagattcaaaaccaaattgaagaagtaaaaaacgaaggtttatataaatcagaacgtattattacttcagctcaaaaagcatcagtttctatttctactggccaagaagtactaaatttctgtgcaaataactacttaggtttagctaaccaccctgctcttattgaagcagcaaaagatggaatggatgagcatggctttggtatggcatcagttcgttttatttgtggtactcaagattcacataaagaattagaacaaaagctatctactttcttaggcaaagaagacaccattctttacacatcatgttttgatgcgaatgctggtttgtttgaaaccattctaggcgcagaagatgcaattatctcagacgcactaaaccacgcatcaatcattgatggtgtgcgtctatgtaaagcaatgcgtttccgctacgcaaacaacaacatgactgaacttgaagaacaattaattgcagcaaaagaagctggcgctcgtcatacgcttatcgtaacggatggtgtgttctcaatggatggcgttgttgcaaaccttcctgcaatctgtgacttagcagataaatacgacgcccttgtgatggttgatgattctcacgcagttggctttatgggcgaaaacggtactggtactcatgagtatcacgatgtaatcgaccgtatcgatatcatcacaggcacacttggtaaagcaatgggtggcgcatcaggcggctacacttcaggtaaaaaagaagtgattgactggcttcgtcagcgttctcgcccttacctattctcaaactctgttgctccggccattgtatctgcatctattcgtgttttagatcttctggctgaatcaggtgacctgcgttctaaattatgggagaactcagcgcatttccgtacacgtatggaagcagcaggctttactatgggcggtgctgatcacgcaattattccaatcatgcttggagatgcaaaaatcgctgttgagtttgctgaacgtgcactagaaaaagggatttatgttattgcattctctttcccagtagtaccaaaaggccaagctcgtattcgtacacaaatgtccgctgctcacactcgtgaacagctagatcgtgcaattgatgcctttatcgaagtaggtaaagacatggagatcattaaataatgaaaattaaagcgctttcaaaattaaaacctgaagaaggtatctggctaactgaggttgaaaaacctgagatgggtcataacgatcttcttatccgcattaagaaaactgccatctgtggtaccgatgtacatatctacaactgggacgaatggtcacaaaaaacaattccagtacctatggttgttggtcacgagtatgtaggtgaagttgttggtattggtcaagaagtgcgtggttttgaaattggagaccgcgtttcaggtgaaggtcacatcacttgtggtcattgtcgtaactgtcgtggcggtcgtactcacctttgtcgtaatacaacaggcgtaggtgtaaaccgtactggtgctttttctgaatacctagttattccagctttcaatgcatttaagatccctgctggtatttcagatgacttagcatcaatctttgacccatttggaaacgcagttcacactgctctatcatttgatttagttggtgaagacgttctaatcactggtgctggtcctattggcatcatggctgcagcagttgcaaagcatgtaggtgctcgtcacgtagttattacagacgtaaatgaataccgcttagaccttgctaagaaaatgggcgtaactcgagcagtaaacgtaatgaacgaaaagcttgaagatgtgatgtctgatcttggtatgacagaaggctttgatgttggtttagaaatgtcaggtaacccatcagcgtttaatagcatgctaactaacatgaaccatggcggtaaaatttctcttctaggtattccaccatcagatatgactgttgattggaaccaagtaatctttaaaggtctagttatcaaaggcatttatggccgtgaaatgtttgaaacctggtataagatggcaagcttaattcaatctggcctagatctaacaccaattatcacgcaccactacaagattgatgacttccaagcaggcttcgatatgatgcgctctggtatgtctggtaaagttattctagattgggaataagtcgactctagaggatccccgggtaccgagctcgaattcagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttggggtgggcgaagaactccagcatgagatccccgcgctggaggatcatccagccattcggggtcgttcactggttcccctttctgatttctggcatagaagaacccccgtgaactgtgtggttccgggggttgctgatttttgcgagacttctcgcgcaattccctagcttaggtgaaaacaccatgaaacactagggaaacacccatgaaacacccattagggcagtagggcggcttcttcgtctagggcttgcatttgggcggtgatctggtctttagcgtgtgaaagtgtgtcgtaggtggcgtgctcaatgcactcgaacgtcacgtcatttaccgggtcacggtgggcaaagagaactagtgggttagacattgttttcctcgttgtcggtggtggtgagcttttctagccgctcggtaaacgcggcgatcatgaactcttggaggttttcaccgttctgcatgcctgcgcgcttcatgtcctcacgtagtgccaaaggaacgcgtgcggtgaccacgacgggcttagcctttgcctgcgcttctagtgcttcgatggtggcttgtgcctgcgcttgctgcgcctgtagtgcctgttgagcttcttgtagttgctgttctagctgtgccttggttgccatgctttaagactctagtagctttcctgcgatatgtcatgcgcatgcgtagcaaacattgtcctgcaactcattcattatgtgcagtgctcctgttactagtcgtacatactcatatttacctagtctgcatgcagtgcatgcacatgcagtcatgtcgtgctaatgtgtaaaacatgtacatgcagattgctgggggtgcagggggcggagccaccctgtccatgcggggtgtggggcttgccccgccggtacagacagtgagcaccggggcacctagtcgcggataccccccctaggtatcggacacgtaaccctcccatgtcgatgcaaatctttaacattgagtacgggtaagctggcacgcatagccaagctaggcggccaccaaacaccactaaaaattaatagtccctagacaagacaaacccccgtgcgagctaccaactcatatgcacgggggccacataacccgaaggggtttcaattgacaaccatagcactagctaagacaacgggcacaacacccgcacaaactcgcactgcgcaaccccgcacaacatcgggtctaggtaacactgagtaacactgaaatagaagtgaacacctctaaggaaccgcaggtcaatgagggttctaaggtcactcgcgctagggcgtggcgtaggcaaaacgtcatgtacaagatcaccaatagtaaggctctggcggggtgccataggtggcgcagggacgaagctgttgcggtgtcctggtcgtctaacggtgcttcgcagtttgagggtctgcaaaactctcactctcgctgggggtcacctctggctgaattggaagtcatgggcgaacgccgcattgagctggctattgctactaagaatcacttggcggcgggtggcgcgctcatgatgtttgtgggcactgttcgacacaaccgctcacagtcatttgcgcaggttgaagcgggtattaagactgcgtactcttcgatggtgaaaacatctcagtggaagaaagaacgtgcacggtacggggtggagcacacctatagtgactatgaggtcacagactcttgggcgaacggttggcacttgcaccgcaacatgctgttgttcttggatcgtccactgtctgacgatgaactcaaggcgtttgaggattccatgttttcccgctggtctgctggtgtggttaaggccggtatggacgcgccactgcgtgagcacggggtcaaacttgatcaggtgtctacctggggtggagacgctgcgaaaatggcaacctacctcgctaagggcatgtctcaggaactgactggctccgctactaaaaccgcgtctaaggggtcgtacacgccgtttcagatgttggatatgttggccgatcaaagcgacgccggcgaggatatggacgctgttttggtggctcggtggcgtgagtatgaggttggttctaaaaacctgcgttcgtcctggtcacgtggggctaagcgtgctttgggcattgattacatagacgctgatgtacgtcgtgaaatggaagaagaactgtacaagctcgccggtctggaagcaccggaacgggtcgaatcaacccgcgttgctgttgctttggtgaagcccgatgattggaaactgattcagtctgatttcgcggttaggcagtacgttctcgattgcgtggataaggctaaggacgtggccgctgcgcaacgtgtcgctaatgaggtgctggcaagtctgggtgtggattccaccccgtgcatgatcgttatggatgatgtggacttggacgcggttctgcctactcatggggacgctactaagcgtgatctgaatgcggcggtgttcgcgggtaatgagcagactattcttcgcacccactaaaagcggcataaaccccgttcgatattttgtgcgatgaatttatggtcaatgtcgcgggggcaaactatgatgggtcttgttgttggcgtcccggaaaacgattccgaagcccaacctttcatagaaggcggcggtggaatcgaaatctcgtgatggcaggttgggcgtcgcttggtcggtcatttcgaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgtcgaagctcggcggatttgtcctactcaagctgatccgacaaaatccacacattatcccaggtgtccggatcggtcaaatacgctgccagctcatagaccgtatccaaagcatccggggctgatccccggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcgatggtgtcaacgtaaatgccgcttcgccttcgcgcgcgaattgcaagctgatccgggcttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaat

SEQ ID NO: 20 - DNA, 8840 bp, synthetic construct
  source 1..8840: mol_type = other DNA
gagctgttgacaattaatcatcggctcgtataatgtgtggaattgtgagcggataacaatttcacacaggaaacagaattaattaagcttaatgaaaaatatggcatcacaaaaaatgaaagcattggtaaaaaaatacaatcagccaggattgtggatggagaaagtggatatccccagcattgggatcgacgatgttctgattaaaattcacaaaaccgccatttgcggaactgacatccacatttataactgggatgaatgggcgcagaaaaccattccctacccaatggtggtcggtcatgaatatgttggcacgattgccgcaattggaagcaatgtcaaaggttttgagattggcgaagttgtcagcggggaggggcatattgtgtgcggccattgtcgcaactgtcgggccggacgccgccatctttgcatgaatacccgtggagtgggagtaaaccgtcaaggatcgttcgccgaattcttatctatccctgccagtaatgtatggcgctgtgatcccaacattccccaagacgttctggcgtgttttgatccctttggcaacgccgtccatacgaccctttcttatgatctggtaggtgaagatgtgctcatcacaggggccggccccattggtattatggccgtcgccatcgcccgtcatgcaggggcccgtcatgtcgttatcacggacgtcaatccctaccgccttggtctggcaaaaaaaatgggagccacccgagcggttaatgttcaagaagaaaaattatcaaacgtcattaaagatctaaaaatgactgaaggttttgatatcgggcttgagatgtcggggaacgttcaggcctttaaagatatgatcgaccatatggttcacggtggaaaaatcgccctgcttggtattctgccaaacggcacgggcattgattgggacaaagtggtcttcaacagtctcaatatcaaaggtatttacggtcgtgaaatgtttgaaacctggtatcagatgacagttctcttgcaatcgggactagatattagtcctgtcattacccatcgttttcccattgacgattttgaaaaaggttttgaactgatgaagtcaggtcagtcaggaaaagtaattttaaactggatttaattatgtttaaccaaatgaaagattatttcatcaaagagatggccgaaattaaagagcaaggattgtataaatcggaacgccttatcaacaccccccaaagttcccatatcggagttgtcggaggacaggaagttattaatctctgcgccaataattatttgggcctggcaaatcatcccgatgtcctcaaagccgccaaggcaagctacgataagtggggctatggactttcttcggtgcgatttatctgtggcactcaggaaatccacaaaactttagaacgcaaaatttctgaatttctgggaactgaggacaccattctttattcatcatgctttgatgccaatggtggactttttgaaacaatcctgggtgaagaagatgccatcatcagcgatgaactcaaccatgccagcattattgacggtattcgcctttgtaaggccaagcgctttaaatataaaaataatgatcccaatgatttagaatcaaaactcaaagaagctgaccagtccggcgcgcgttttaaaatggttgccagcgatggtgttttctccatggatggatatattgctaacctcaaagcagtatgtgaactggcccagaagtatcagtgtttggtgatggtggatgattctcatgccgtgggattcatcgggcaaaatggtcgaggcacccacgaacattgcaaagttatgggtcaagttgatatcatcaccggcaccctcggtaaggccttgggaggtgcgagcggaggctacaccagcgggcgcaaagaaatcatcgaaaagttacgtcaacgttcacgcccttacttattttccaataccctcgctcccagtatcgtctgtgcttctttgaaagttttggaactgttaagtcagagcgatgaattgcgaaccaaactacgacagaacacccaatttttccgtacagaaatgaccaaggccggttttaacatcttaaaaggcgaacatccgatcgtgcctgttatgttaggtgacgccattctggcacagaaaattgctgcgcgcttattagaattgggagtctatgtcgttggcttttatttcccggttgttcccaagggacaggcccgcatccgcacccaagtttctgccgcccactcgcgcacagatttagaattcgccatcgcccaatttattcaagtgaagcgtgaattctcagcaggagtataagtcgactctagaggatccccgggtaccgagctcgaattcagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttggggtgggcgaagaactccagcatgagatccccgcgctggaggatcatccagccattcggggtcgttcactggttcccctttctgatttctggcatagaagaacccccgtgaactgtgtggttccgggggttgctgatttttgcgagacttctcgcgcaattccctagcttaggtgaaaacaccatgaaacactagggaaacacccatgaaacacccattagggcagtagggcggcttcttcgtctagggcttgcatttgggcggtgatctggtctttagcgtgtgaaagtgtgtcgtaggtggcgtgctcaatgcactcgaacgtcacgtcatttaccgggtcacggtgggcaaagagaactagtgggttagacattgttttcctcgttgtcggtggtggtgagcttttctagccgctcggtaaacgcggcgatcatgaactcttggaggttttcaccgttctgcatgcctgcgcgcttcatgtcctcacgtagtgccaaaggaacgcgtgcggtgaccacgacgggcttagcctttgcctgcgcttctagtgcttcgatggtggcttgtgcctgcgcttgctgcgcctgtagtgcctgttgagcttcttgtagttgctgttctagctgtgccttggttgccatgctttaagactctagtagctttcctgcgatatgtcatgcgcatgcgtagcaaacattgtcctgcaactcattcattatgtgcagtgctcctgttactagtcgtacatactcatatttacctagtctgcatgcagtgcatgcacatgcagtcatgtcgtgctaatgtgtaaaacatgtacatgcagattgctgggggtgcagggggcggagccaccctgtccatgcggggtgtggggcttgccccgccggtacagacagtgagcaccggggcacctagtcgcggataccccccctaggtatcggacacgtaaccctcccatgtcgatgcaaatctttaacattgagtacgggtaagctggcacgcatagccaagctaggcggccaccaaacaccactaaaaattaatagtccctagacaagacaaacccccgtgcgagctaccaactcatatgcacgggggccacataacccgaaggggtttcaattgacaaccatagcactagctaagacaacgggcacaacacccgcacaaactcgcactgcgcaaccccgcacaacatcgggtctaggtaacactgagtaacactgaaatagaagtgaacacctctaaggaaccgcaggtcaatgagggttctaaggtcactcgcgctagggcgtggcgtaggcaaaacgtcatgtacaagatcaccaatagtaaggctctggcggggtgccataggtggcgcagggacgaagctgttgcggtgtcctggtcgtctaacggtgcttcgcagtttgagggtctgcaaaactctcactctcgctgggggtcacctctggctgaattggaagtcatgggcgaacgccgcattgagctggctattgctactaagaatcacttggcggcgggtggcgcgctcatgatgtttgtgggcactgttcgacacaaccgctcacagtcatttgcgcaggttgaagcgggtattaagactgcgtactcttcgatggtgaaaacatctcagtggaagaaagaacgtgcacggtacggggtggagcacacctatagtgactatgaggtcacagactcttgggcgaacggttggcacttgcaccgcaacatgctgttgttcttggatcgtccactgtctgacgatgaactcaaggcgtttgaggattccatgttttcccgctggtctgctggtgtggttaaggccggtatggacgcgccactgcgtgagcacggggtcaaacttgatcaggtgtctacctggggtggagacgctgcgaaaatggcaacctacctcgctaagggcatgtctcaggaactgactggctccgctactaaaaccgcgtctaaggggtcgtacacgccgtttcagatgttggatatgttggccgatcaaagcgacgccggcgaggatatggacgctgttttggtggctcggtggcgtgagtatgaggttggttctaaaaacctgcgttcgtcctggtcacgtggggctaagcgtgctttgggcattgattacatagacgctgatgtacgtcgtgaaatggaagaagaactgtacaagctcgccggtctggaagcaccggaacgggtcgaatcaacccgcgttgctgttgctttggtgaagcccgatgattggaaactgattcagtctgatttcgcggttaggcagtacgttctcgattgcgtggataaggctaaggacgtggccgctgcgcaacgtgtcgctaatgaggtgctggcaagtctgggtgtggattccaccccgtgcatgatcgttatggatgatgtggacttggacgcggttctgcctactcatggggacgctactaagcgtgatctgaatgcggcggtgttcgcgggtaatgagcagactattcttcgcacccactaaaagcggcataaaccccgttcgatattttgtgcgatgaatttatggtcaatgtcgcgggggcaaactatgatgggtcttgttgttggcgtcccggaaaacgattccgaagcccaacctttcatagaaggcggcggtggaatcgaaatctcgtgatggcaggttgggcgtcgcttggtcggtcatttcgaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgtcgaagctcggcggatttgtcctactcaagctgatccgacaaaatccacacattatcccaggtgtccggatcggtcaaatacgctgccagctcatagaccgtatccaaagcatccggggctgatccccggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcgatggtgtcaacgtaaatgccgcttcgccttcgcgcgcgaattgcaagctgatccgggcttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaat

SEQ ID NO: 21 - DNA, 8812 bp, synthetic construct
  source 1..8812: mol_type = other DNA
gagctgttgacaattaatcatcggctcgtataatgtgtggaattgtgagcggataacaatttcacacaggaaacagaattaattaagcttaatttgccctgtctggagaatcgcaatgtcagcgctggctcagctgcgtggcatcctggaggaggagctggaaagcatccgcggagctggcacctggaagagcgagcgggtcatcacgtcccgtcaggggccgcacatccacgtggacggcgccccgggaggaattattaacttctgtgccaacaactacctgggcctgagcagccaccctgaggtcattcaggcaggtctgcggaccctaaaggagtttggagccggccttagctcagtccgcttcatctgtggcacccagagcatccacaaagacctagaagcaaagatcgcccgctttcaccaacgcgaggatgccatcctctatcccagctgttttgatgccaacaccggcctctttgaggcactgctgacttcggaggacgcagtcctgtcggatgagctgaaccatgcctccatcatcgatggcatccgtctgtgcaaggcccacaagtaccgctaccgccacctggatatggctgatctggaggccaagctgcaggaggcccagaagcatcggctgcgcctggtggccactgatggggccttttccatggatggtgacatcgcacccctgcaggagatctgccgccttgcctctcaatatggtgccttagtctttgtggacgaaagccacgccactggcttcctgggggccacaggacggggcacagatgagctgctgggcgtgatggaccaagtcaccatcatcaactccacactggggaaggccctgggtggagcatcagggggctacacgactgggcctggggccctggtatccctgctacggcagcgtgcccggccctacctcttctccaacagcctgccccctgctgctgtcggctgtgcctccaaggccctggacctgctcatggagagcaacgccatcgtccagtctatggcggccaagaccctgcgattccgcagtcagatggaggctgctggcttcaccatctcaggagccaatcaccccatctgccctgtgatgctgggtgacgctcggctggccttaaacattgcggatgacatgcttaagcgtggcatctttgtcattgggttcagctaccctgtggtccccaaaggcaaggcccgtatccgagtccagatctcagcagtgcacagtgaggaggacattgaccgctgtgtggaggccttcgtggaggtggggcgactgcacggcgcgctgccttgaacgtacttaggaaggagatatagatatggaagcagaccagcagcgtgtcttaattacagggggtcttggccagcttggagtgggtcttgctagctttctgcgcaaacgatttgggaaggacaatgtgattctatctgacattcggaagcccccggaacacgtctttcttagtggcccgtttatttattccgacatcctggattacaagaatcttcgggagattgtggtgaacaaccgcatcacgtggctgttccattacagcgccctgctcagcgcagtgggagaagcaaacgtgtccctggcccgtgccgtgaacatcactggactgcacaatgtcctggacgttgccgcagagcacggtctgcgattgtttgtgccaagcaccatcggggcgtttggacctacttctccacggaacccaacccctgacctctgcatccagcgtccccgcaccatctatggggtgtccaaggtccacgctgagcttatgggcgagtattattattaccggtatggattagactttcggtgcctgcgctatcctggaatcatttctgctgattcccagcctggaggaggaacaactgactatgcggtccagattttccatgaagctgtcaagaatggccgttttgagtgcaacctgaaacccgacacgcgcctcccaatgatgtacattgacgactgcctccgggccaccctggaggtcatggaggccccagcagagtccctgtccatgcgtacctacaacatcagcgccatgagcttcacccccgaggagctggcccaggaggtgctcaagcatgtcccagaactccaggtcacctacaatgttgatcctgtccgacaggccatcgcggatagttggccaatgaactttgatgatagcaacgctcggaaggactggggatggaagcacgattttgacctcccagagctggtgacaacgatgttgaacttccacggttctgagagccgcgttgcccaggctaactgagtcgactctagaggatccccgggtaccgagctcgaattcagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttggggtgggcgaagaactccagcatgagatccccgcgctggaggatcatccagccattcggggtcgttcactggttcccctttctgatttctggcatagaagaacccccgtgaactgtgtggttccgggggttgctgatttttgcgagacttctcgcgcaattccctagcttaggtgaaaacaccatgaaacactagggaaacacccatgaaacacccattagggcagtagggcggcttcttcgtctagggcttgcatttgggcggtgatctggtctttagcgtgtgaaagtgtgtcgtaggtggcgtgctcaatgcactcgaacgtcacgtcatttaccgggtcacggtgggcaaagagaactagtgggttagacattgttttcctcgttgtcggtggtggtgagcttttctagccgctcggtaaacgcggcgatcatgaactcttggaggttttcaccgttctgcatgcctgcgcgcttcatgtcctcacgtagtgccaaaggaacgcgtgcggtgaccacgacgggcttagcctttgcctgcgcttctagtgcttcgatggtggcttgtgcctgcgcttgctgcgcctgtagtgcctgttgagcttcttgtagttgctgttctagctgtgccttggttgccatgctttaagactctagtagctttcctgcgatatgtcatgcgcatgcgtagcaaacattgtcctgcaactcattcattatgtgcagtgctcctgttactagtcgtacatactcatatttacctagtctgcatgcagtgcatgcacatgcagtcatgtcgtgctaatgtgtaaaacatgtacatgcagattgctgggggtgcagggggcggagccaccctgtccatgcggggtgtggggcttgccccgccggtacagacagtgagcaccggggcacctagtcgcggataccccccctaggtatcggacacgtaaccctcccatgtcgatgcaaatctttaacattgagtacgggtaagctggcacgcatagccaagctaggcggccaccaaacaccactaaaaattaatagtccctagacaagacaaacccccgtgcgagctaccaactcatatgcacgggggccacataacccgaaggggtttcaattgacaaccatagcactagctaagacaacgggcacaacacccgcacaaactcgcactgcgcaaccccgcacaacatcgggtctaggtaacactgagtaacactgaaatagaagtgaacacctctaaggaaccgcaggtcaatgagggttctaaggtcactcgcgctagggcgtggcgtaggcaaaacgtcatgtacaagatcaccaatagtaaggctctggcggggtgccataggtggcgcagggacgaagctgttgcggtgtcctggtcgtctaacggtgcttcgcagtttgagggtctgcaaaactctcactctcgctgggggtcacctctggctgaattggaagtcatgggcgaacgccgcattgagctggctattgctactaagaatcacttggcggcgggtggcgcgctcatgatgtttgtgggcactgttcgacacaaccgctcacagtcatttgcgcaggttgaagcgggtattaagactgcgtactcttcgatggtgaaaacatctcagtggaagaaagaacgtgcacggtacggggtggagcacacctatagtgactatgaggtcacagactcttgggcgaacggttggcacttgcaccgcaacatgctgttgttcttggatcgtccactgtctgacgatgaactcaaggcgtttgaggattccatgttttcccgctggtctgctggtgtggttaaggccggtatggacgcgccactgcgtgagcacggggtcaaacttgatcaggtgtctacctggggtggagacgctgcgaaaatggcaacctacctcgctaagggcatgtctcaggaactgactggctccgctactaaaaccgcgtctaaggggtcgtacacgccgtttcagatgttggatatgttggccgatcaaagcgacgccggcgaggatatggacgctgttttggtggctcggtggcgtgagtatgaggttggttctaaaaacctgcgttcgtcctggtcacgtggggctaagcgtgctttgggcattgattacatagacgctgatgtacgtcgtgaaatggaagaagaactgtacaagctcgccggtctggaagcaccggaacgggtcgaatcaacccgcgttgctgttgctttggtgaagcccgatgattggaaactgattcagtctgatttcgcggttaggcagtacgttctcgattgcgtggataaggctaaggacgtggccgctgcgcaacgtgtcgctaatgaggtgctggcaagtctgggtgtggattccaccccgtgcatgatcgttatggatgatgtggacttggacgcggttctgcctactcatggggacgctactaagcgtgatctgaatgcggcggtgttcgcgggtaatgagcagactattcttcgcacccactaaaagcggcataaaccccgttcgatattttgtgcgatgaatttatggtcaatgtcgcgggggcaaactatgatgggtcttgttgttggcgtcccggaaaacgattccgaagcccaacctttcatagaaggcggcggtggaatcgaaatctcgtgatggcaggttgggcgtcgcttggtcggtcatttcgaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgtcgaagctcggcggatttgtcctactcaagctgatccgacaaaatccacacattatcccaggtgtccggatcggtcaaatacgctgccagctcatagaccgtatccaaagcatccggggctgatccccggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcgatggtgtcaacgtaaatgccgcttcgccttcgcgcgcgaattgcaagctgatccgggcttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaat

SEQ ID NO: 22 - DNA, 8898 bp, synthetic construct
  source 1..8898: mol_type = other DNA
gagctgttgacaattaatcatcggctcgtataatgtgtggaattgtgagcggataacaatttcacacaggaaacagaattaattaagctttcccgcccgacgaaaggacgcccgcatgcgtgaagatttctacggccgggttcgcagcgaactcgccgacatcgatgcccagggcctgaccaagcccgagcgcgtgatcacctcccgccagggcgccgacatccgtgtgaagctgccgggtggcggtgaacgtgaggtgctgaacttctgcgccaacaactatctgggcctggccggcgacgaacgcgtgacctgcgccgcgattgaggcttccgagcgttggggtgccggtacggcgtctgttcgtttcatctgcggcaccatggacatccacaagcagctggagcgggccatcgccgactacctcggctatgaggactccatcctgttcgcggccgccttcgacgccaacggcggggtgttcgagccgttgctgggcgaggaagacgccatcgtctccgacagcctgaaccacgcctcgatcatcgacggcgtgcgcctgtgcaaggccaagcgctatcgcttcgccaacagcgacatggccgatctggaagccaagctggccgaggccaaggccgcaggcgcacgtcagatcatgatcgccaccgacggcgccttctcgatggacggctacatcgccaagctggaccagctgtgcgcgctcgccgaccagtacgacgccctggtcatggtcgacgactgccacgccaccggcttcctcggtccacagggccgtggttcgggtgcatggaacggtgtggagaagcgcgtcgacatcgtcaccggcaccttcggcaaggcgctgggtggggccatgggtggcttcatctgtgcacgcaaggaagtggtcgacctgctgcgccagcgcgcacgtccgtacctgttctccaacgcgctgtcgccggccgtctgcggtgccagcctggaagcgatccgcatcgcagcaggttcggaaggcgacgcgttgcgcaagaagctgttcgacaacgccgcccgcttccgtgcaggcatggccgcggcaggcttcaagctgctggaaggcgaacatccgatcatcccggtgatgctgggcgacgccaagctggcccaggacatggccgccaagctgctggaggagggcatctacgtgatcggcttctccttcccggtggtgccgcgtggtgccgcacgtatccgcacccagatgtccgcggcccatgagccgcaccacgtcgacgccgcgatcgcagcattcaccaaggtcggccgtgaattgggcgtgatctcctgatcaagtagaggttctcccatgacgtcgaccatgaaggcgctggccaagatgaagccggccgagggcctggagctgatcgaggctccgatcccgaccccgggcccagaggacgttctgatccgcgtgaagcggaccgccatctgcggcactgacatccacatctggaactgggacgagtggtcccagaagaacgtgccggtgccgatgatcaccggccacgagttctccggcgagatcgtcgagatcggcaaggacgtcgaccgtccgctgaagatcggccagcgcgtctcggccgaaggccacgtcatcgacctgaacagcttcgccgcccgggcaggtcacttccacctggacccgaatacccgcggcatcggcgtgaaccggcagggtgccttcgccgagtacgtggtggccccagcattcaacgtgatcccgctgcccgacgacgtgtcgttcgaggtggcgtccatgctcgacccgttcggcaacgccgtgcacaccgcccagcagttcgacttggttggcgaggacgtgctggtcaccggggcaggtccgatcggtatcatggccgcagccgtggcacgtcgcgcaggtgcccgcactgttgtgctgaccgacgtcaacgagtaccgcctggacctggcccgcaaggttgccgacgtgcgcactgtcaacgtcgccaaggaagacctcaaggacgtcatgaaggagctgggcatccaggtcggcttcgacgtggctctggagatgtcgggcgcgccgtcggccttcaaccagtgcgtcgacaacctgatcatgggcggcgggctggccctgttgggcatcccgtcccgtccaatggagaccgactggggcaagatcatcctcaaggccctgaccatcaaaggggtctacggccgcgagatgttcgacacctggcgcaagatgctgggcctgctgaaggccggcgtcgacctgaccccgctgatcacccaccggctggcttacgaccgctacgtcgaaggctttgaggcgatgaagtcgggccgctcgggcaaggtcgtgctcgactgggacaaggctgcgtaagtcgactctagaggatccccgggtaccgagctcgaattcagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttggggtgggcgaagaactccagcatgagatccccgcgctggaggatcatccagccattcggggtcgttcactggttcccctttctgatttctggcatagaagaacccccgtgaactgtgtggttccgggggttgctgatttttgcgagacttctcgcgcaattccctagcttaggtgaaaacaccatgaaacactagggaaacacccatgaaacacccattagggcagtagggcggcttcttcgtctagggcttgcatttgggcggtgatctggtctttagcgtgtgaaagtgtgtcgtaggtggcgtgctcaatgcactcgaacgtcacgtcatttaccgggtcacggtgggcaaagagaactagtgggttagacattgttttcctcgttgtcggtggtggtgagcttttctagccgctcggtaaacgcggcgatcatgaactcttggaggttttcaccgttctgcatgcctgcgcgcttcatgtcctcacgtagtgccaaaggaacgcgtgcggtgaccacgacgggcttagcctttgcctgcgcttctagtgcttcgatggtggcttgtgcctgcgcttgctgcgcctgtagtgcctgttgagcttcttgtagttgctgttctagctgtgccttggttgccatgctttaagactctagtagctttcctgcgatatgtcatgcgcatgcgtagcaaacattgtcctgcaactcattcattatgtgcagtgctcctgttactagtcgtacatactcatatttacctagtctgcatgcagtgcatgcacatgcagtcatgtcgtgctaatgtgtaaaacatgtacatgcagattgctgggggtgcagggggcggagccaccctgtccatgcggggtgtggggcttgccccgccggtacagacagtgagcaccggggcacctagtcgcggataccccccctaggtatcggacacgtaaccctcccatgtcgatgcaaatctttaacattgagtacgggtaagctggcacgcatagccaagctaggcggccaccaaacaccactaaaaattaatagtccctagacaagacaaacccccgtgcgagctaccaactcatatgcacgggggccacataacccgaaggggtttcaattgacaaccatagcactagctaagacaacgggcacaacacccgcacaaactcgcactgcgcaaccccgcacaacatcgggtctaggtaacactgagtaacactgaaatagaagtgaacacctctaaggaaccgcaggtcaatgagggttctaaggtcactcgcgctagggcgtggcgtaggcaaaacgtcatgtacaagatcaccaatagtaaggctctggcggggtgccataggtggcgcagggacgaagctgttgcggtgtcctggtcgtctaacggtgcttcgcagtttgagggtctgcaaaactctcactctcgctgggggtcacctctggctgaattggaagtcatgggcgaacgccgcattgagctggctattgctactaagaatcacttggcggcgggtggcgcgctcatgatgtttgtgggcactgttcgacacaaccgctcacagtcatttgcgcaggttgaagcgggtattaagactgcgtactcttcgatggtgaaaacatctcagtggaagaaagaacgtgcacggtacggggtggagcacacctatagtgactatgaggtcacagactcttgggcgaacggttggcacttgcaccgcaacatgctgttgttcttggatcgtccactgtctgacgatgaactcaaggcgtttgaggattccatgttttcccgctggtctgctggtgtggttaaggccggtatggacgcgccactgcgtgagcacggggtcaaacttgatcaggtgtctacctggggtggagacgctgcgaaaatggcaacctacctcgctaagggcatgtctcaggaactgactggctccgctactaaaaccgcgtctaaggggtcgtacacgccgtttcagatgttggatatgttggccgatcaaagcgacgccggcgaggatatggacgctgttttggtggctcggtggcgtgagtatgaggttggttctaaaaacctgcgttcgtcctggtcacgtggggctaagcgtgctttgggcattgattacatagacgctgatgtacgtcgtgaaatggaagaagaactgtacaagctcgccggtctggaagcaccggaacgggtcgaatcaacccgcgttgctgttgctttggtgaagcccgatgattggaaactgattcagtctgatttcgcggttaggcagtacgttctcgattgcgtggataaggctaaggacgtggccgctgcgcaacgtgtcgctaatgaggtgctggcaagtctgggtgtggattccaccccgtgcatgatcgttatggatgatgtggacttggacgcggttctgcctactcatggggacgctactaagcgtgatctgaatgcggcggtgttcgcgggtaatgagcagactattcttcgcacccactaaaagcggcataaaccccgttcgatattttgtgcgatgaatttatggtcaatgtcgcgggggcaaactatgatgggtcttgttgttggcgtcccggaaaacgattccgaagcccaacctttcatagaaggcggcggtggaatcgaaatctcgtgatggcaggttgggcgtcgcttggtcggtcatttcgaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgtcgaagctcggcggatttgtcctactcaagctgatccgacaaaatccacacattatcccaggtgtccggatcggtcaaatacgctgccagctcatagaccgtatccaaagcatccggggctgatccccggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcgatggtgtcaacgtaaatgccgcttcgccttcgcgcgcgaattgcaagctgatccgggcttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaat

SEQ ID NO: 23 - DNA, 8771 bp, synthetic construct
  source 1..8771: mol_type = other DNA
gagctgttgacaattaatcatcggctcgtataatgtgtggaattgtgagcggataacaatttcacacaggaaacagaattaattaagcttctcgcacaccaacgggagccaagccatgtcgaatgccgaggcattctatgcatccatccgtaccgaactcgaaagcatccgcgcggcggggctgttcaagaacgagcgcgtgatcgccacgccgcagggtgcacgggtgcgcaccaccgatggtcgcgaggtcatcaacctgtgcgccaacaactaccttggcttgtcgtcgcacccgcaggtgatcgaggccgcgcatgaagcgctgcgcacgcatggcttcgggctgagctcggtgcgctttatctgcggcacgcaagacctgcacaagacgctggaggcgcggctgtcggccttcctcggcaccgaggacaccatcctctatggctcggccttcgatgccaatggcggtttgttcgaaacgctgctgggcgcggaagacgcggtcatcagcgatgcgctcaaccatgcctcgatcatcgacggcgtccgcctgtccaaggcgcgccgctaccgctaccagcacaacgacatggacgacctgcgcgtgcaactggagcaggcgcgggccgatggtgcacgctacacgctggtgttttccgacggcgtgttctcgatggacggcaccgtggcgcggctcgacgagatgcgcgcgatctgcgacgagtacggcgcattgttgggcatcgatgaatgccatgccaccggtttcatgggccagcgcgggcgtggtacgcatgaggcgcgcggtgtgttcggcaagatcgacatcattaccggcacgctgggcaaggcgctgggtggcgcctctggtggtttcaccagcgcgcgcaaggaagtggtggcactgctgcgccagcgttcgcggccgtatttgttctccaacaccgtggcgccggccatcgtgggtgcgtcgatcgcggtgctcgatattctggaagccagcaccgagctgcgcgaccggctggagggcaatacccgcttcttccgcgcgggactggaccggttgggcttcgacgtcaaggcgggcgaccacccgatcatcccgatcatggtctacgacgccgacaaggcgcagcaactggcgcagcgcctgctggagctgggcgtgtacgtggtcggcttcttctacccggtcgtacccaagggccaggcgcgcatccgcgtgcagatgagcgcgctgcatgacgaggcagcactgcaggcggcgctggatgccttcggccaggctggccgtgaactggggctgatctgatggaagctggcaaaccgaagatcctgattgtcggcgccaacggccagatcgggtctgaactggcactggcgctggccgagcgctacgggcgcaccaacgtgatcacctccgacgtggtgcccaccggtcgtcatgtgcatctgacgcacgagatgctcaacgccaccgaccgcggcgagctggccaccgtggtcgaacgtcatggcatcacccaggtctacctgctggccgccgcgctgtctgccactggcgaaaaggcaccacagtgggcctggaacctcaatatgaccagcctgctcaatgtgctggagctggcgcggcagaccgggctggagcgggtgttctggccaagctcgattgcagccttcggcccgaccacgcctgcaggacagacaccgcagaagaccgtgatggagcccaccacggtctacggcatctccaagcaggcgggcgagggttggtgccgctggtatcacgccaaccacggcgtggatgtgcgcagcgttcgctatccgggcctgatctcgcacaagacgccacccggcggcggaaccaccgactatgcggtcgatatcttccatgcggcggtgacgggcgagccatacacctgcttcctgaaggaagacgaagccctgccgatgatgtatatgcccgatgcgatccgcgccaccatcgaactgatggaagccccggcggacaagctgagcgagcgcggcagctacaacatcgccggcatgagcttcacgcccgcgcagatcgccgcagccatccgcgagcaggttccaggcttccagatccgctatgaacccgactatcgccaggcgattgcgcagggctggccggattcgatcgatgattcggtcgcgcgcgcggattgggggtggaaggcccagtatggactgaaggagatggtcgcggacatgcttgccaacctgaaggccacgctggcgggctgagtcgactctagaggatccccgggtaccgagctcgaattcagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttggggtgggcgaagaactccagcatgagatccccgcgctggaggatcatccagccattcggggtcgttcactggttcccctttctgatttctggcatagaagaacccccgtgaactgtgtggttccgggggttgctgatttttgcgagacttctcgcgcaattccctagcttaggtgaaaacaccatgaaacactagggaaacacccatgaaacacccattagggcagtagggcggcttcttcgtctagggcttgcatttgggcggtgatctggtctttagcgtgtgaaagtgtgtcgtaggtggcgtgctcaatgcactcgaacgtcacgtcatttaccgggtcacggtgggcaaagagaactagtgggttagacattgttttcctcgttgtcggtggtggtgagcttttctagccgctcggtaaacgcggcgatcatgaactcttggaggttttcaccgttctgcatgcctgcgcgcttcatgtcctcacgtagtgccaaaggaacgcgtgcggtgaccacgacgggcttagcctttgcctgcgcttctagtgcttcgatggtggcttgtgcctgcgcttgctgcgcctgtagtgcctgttgagcttcttgtagttgctgttctagctgtgccttggttgccatgctttaagactctagtagctttcctgcgatatgtcatgcgcatgcgtagcaaacattgtcctgcaactcattcattatgtgcagtgctcctgttactagtcgtacatactcatatttacctagtctgcatgcagtgcatgcacatgcagtcatgtcgtgctaatgtgtaaaacatgtacatgcagattgctgggggtgcagggggcggagccaccctgtccatgcggggtgtggggcttgccccgccggtacagacagtgagcaccggggcacctagtcgcggataccccccctaggtatcggacacgtaaccctcccatgtcgatgcaaatctttaacattgagtacgggtaagctggcacgcatagccaagctaggcggccaccaaacaccactaaaaattaatagtccctagacaagacaaacccccgtgcgagctaccaactcatatgcacgggggccacataacccgaaggggtttcaattgacaaccatagcactagctaagacaacgggcacaacacccgcacaaactcgcactgcgcaaccccgcacaacatcgggtctaggtaacactgagtaacactgaaatagaagtgaacacctctaaggaaccgcaggtcaatgagggttctaaggtcactcgcgctagggcgtggcgtaggcaaaacgtcatgtacaagatcaccaatagtaaggctctggcggggtgccataggtggcgcagggacgaagctgttgcggtgtcctggtcgtctaacggtgcttcgcagtttgagggtctgcaaaactctcactctcgctgggggtcacctctggctgaattggaagtcatgggcgaacgccgcattgagctggctattgctactaagaatcacttggcggcgggtggcgcgctcatgatgtttgtgggcactgttcgacacaaccgctcacagtcatttgcgcaggttgaagcgggtattaagactgcgtactcttcgatggtgaaaacatctcagtggaagaaagaacgtgcacggtacggggtggagcacacctatagtgactatgaggtcacagactcttgggcgaacggttggcacttgcaccgcaacatgctgttgttcttggatcgtccactgtctgacgatgaactcaaggcgtttgaggattccatgttttcccgctggtctgctggtgtggttaaggccggtatggacgcgccactgcgtgagcacggggtcaaacttgatcaggtgtctacctggggtggagacgctgcgaaaatggcaacctacctcgctaagggcatgtctcaggaactgactggctccgctactaaaaccgcgtctaaggggtcgtacacgccgtttcagatgttggatatgttggccgatcaaagcgacgccggcgaggatatggacgctgttttggtggctcggtggcgtgagtatgaggttggttctaaaaacctgcgttcgtcctggtcacgtggggctaagcgtgctttgggcattgattacatagacgctgatgtacgtcgtgaaatggaagaagaactgtacaagctcgccggtctggaagcaccggaacgggtcgaatcaacccgcgttgctgttgctttggtgaagcccgatgattggaaactgattcagtctgatttcgcggttaggcagtacgttctcgattgcgtggataaggctaaggacgtggccgctgcgcaacgtgtcgctaatgaggtgctggcaagtctgggtgtggattccaccccgtgcatgatcgttatggatgatgtggacttggacgcggttctgcctactcatggggacgctactaagcgtgatctgaatgcggcggtgttcgcgggtaatgagcagactattcttcgcacccactaaaagcggcataaaccccgttcgatattttgtgcgatgaatttatggtcaatgtcgcgggggcaaactatgatgggtcttgttgttggcgtcccggaaaacgattccgaagcccaacctttcatagaaggcggcggtggaatcgaaatctcgtgatggcaggttgggcgtcgcttggtcggtcatttcgaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgtcgaagctcggcggatttgtcctactcaagctgatccgacaaaatccacacattatcccaggtgtccggatcggtcaaatacgctgccagctcatagaccgtatccaaagcatccggggctgatccccggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcgatggtgtcaacgtaaatgccgcttcgccttcgcgcgcgaattgcaagctgatccgggcttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaat

SEQ ID NO: 24 - DNA, 8954 bp, synthetic construct
  source 1..8954: mol_type = other DNA
gagctgttgacaattaatcatcggctcgtataatgtgtggaattgtgagcggataacaatttcacacaggaaacagaattaattaagcttacgaccgacgaggagttctgccgcgatgatgcgtgcactgcgcaagaccgagcccggtcccgggctgaccctgaccgaggtcccagaaccggcctgcggaccatgggacgtgaagatccgggtcctgcgtgcagggatctgcggcacagacctgcacatcctgggctgggacgattccgcggaggcgatgtgcgacacggtcccattcaccccaggacacgagttctacggcgaggtcgtcgaggcaggttccgaggtccccgacgctcgcatcggcgaccgggtctccggagagggacatgtcgtgtgcggggtgtgccgcaactgtcgtgccggacgcaagcagatgtgcatccgcacccgcagcgtcggagtacagcgcgacggagccttcgcagaatacgtcgtgatcccccatctgaacgtgtgggtgcacgagcacgacagccgcaccgaggtgatcaccccggagctgggcgcgttgttcgaccccttcggcaacgccgtccatacggcgctgaagttccccctcgtcggggaggacgtgctgatcaccggcgccggaccgatcggactgatggccgcagcagtagcacggcatgcaggagcacgctatgtcgcgatcaccgatgtccacgagcatcgcctggagatggcccgagccggcggtgtggatctcgccctgaacgtcgccaccaccacgatgacccaggcgcagcgcgaactcggcatgaaggagggcttcgacgtgggactggagatctccggccaggccagcgccctacaggagatgatctcgaacatgaaccacggcggccgtatcgccctgctcggcctgccagcacgacacttcgagctggactggaccaccgtcatcacccacatgctcaccctgcagggcatctacggccgggagatgtacgagacctggaacgcgatgagcgccatgatctccaccagcccgctgctgcgcgaacgggtgctcgccaccgtctccgacgtcgtcccggcatcgcaatgggagtcggcctacgagatcgcacgctccggacgtggaggaaaggtcctgctggactgggagaccatcggctgaagcacatacctacgtcaccaccccctcatccctcgcacttcgatggaaggaccaccgtgtacacggacctcaaggaccagctgaccgccgagctcgccgagatcgagcaggccggcaccttcaagcacgaacgggtcatctcgaccccgcagagcaaccgcatcaccgccgggccgttgggtcgtgacggggcggaagtgctgaacttctgcgccaacaactatctcgggctggccgatcatccggatctgctcgccgcggcgaagaaggcgctggacgagcgcggcttcggcatggcgtcggtgcggttcatctgcggcacccaagatctgcacctcgagctcgagcgggcagtctccaccttcctcggcaccgaggagacgatcctgttctcgtcctgcttcgacgccaacggcgcggtgttcgagccgctgttcggcacgcaggatgcgatcatctccgatgcactgaaccatgcctcgctcatcgacgggatccgcctgtccaaggcagcccggtaccgctaccgcaatgccgatctcgaggacctgcgcacccggctggacgcgacccgtgaccagaacgacggggcaggtgcccgtcgtacgatcatcgtcaccgacggcgtgttctccatggacggctatctcgctcccctcgacggcatctgcgatctcgccgacgaatacggcgccctggtgctggtggacgactcccacgccgtcggtttcatgggcgccactggtgcagggacccccgagcacttcggcgtctcgaaccgcgtggatctctacaccggaaccttcggcaaggcgctgggtggggcaagcggcgggtacgtttctggtcgccgtgagatcgtcgcgatgctgcgccagaaaggtcgtccctacctgttctccaactccctggccccctcgatcgtcgccgccaccctggcagcactcgacctggtcgcaggtagcgccgagctgcgcgcgactctgttccgcaacgcagagctgttccgtcgccggatgagcgaggagggcttcgatctgctggacggcgagcacgcgatcgtgcccgtgatgtttggcgatgcagcactcgcaggtcgcattgcagacgccatgctcgaccacggcgtgttcgtcaccgccttctcctatcccgtggtcccccagggcaaggcccgcatccgcgtgcagctctccgcagcccacacggaagcagacgtcgaggccgcggttcaggcattcatcgcctcacgcgcagctgttcagggctgagtcgactctagaggatccccgggtaccgagctcgaattcagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttggggtgggcgaagaactccagcatgagatccccgcgctggaggatcatccagccattcggggtcgttcactggttcccctttctgatttctggcatagaagaacccccgtgaactgtgtggttccgggggttgctgatttttgcgagacttctcgcgcaattccctagcttaggtgaaaacaccatgaaacactagggaaacacccatgaaacacccattagggcagtagggcggcttcttcgtctagggcttgcatttgggcggtgatctggtctttagcgtgtgaaagtgtgtcgtaggtggcgtgctcaatgcactcgaacgtcacgtcatttaccgggtcacggtgggcaaagagaactagtgggttagacattgttttcctcgttgtcggtggtggtgagcttttctagccgctcggtaaacgcggcgatcatgaactcttggaggttttcaccgttctgcatgcctgcgcgcttcatgtcctcacgtagtgccaaaggaacgcgtgcggtgaccacgacgggcttagcctttgcctgcgcttctagtgcttcgatggtggcttgtgcctgcgcttgctgcgcctgtagtgcctgttgagcttcttgtagttgctgttctagctgtgccttggttgccatgctttaagactctagtagctttcctgcgatatgtcatgcgcatgcgtagcaaacattgtcctgcaactcattcattatgtgcagtgctcctgttactagtcgtacatactcatatttacctagtctgcatgcagtgcatgcacatgcagtcatgtcgtgctaatgtgtaaaacatgtacatgcagattgctgggggtgcagggggcggagccaccctgtccatgcggggtgtggggcttgccccgccggtacagacagtgagcaccggggcacctagtcgcggataccccccctaggtatcggacacgtaaccctcccatgtcgatgcaaatctttaacattgagtacgggtaagctggcacgcatagccaagctaggcggccaccaaacaccactaaaaattaatagtccctagacaagacaaacccccgtgcgagctaccaactcatatgcacgggggccacataacccgaaggggtttcaattgacaaccatagcactagctaagacaacgggcacaacacccgcacaaactcgcactgcgcaaccccgcacaacatcgggtctaggtaacactgagtaacactgaaatagaagtgaacacctctaaggaaccgcaggtcaatgagggttctaaggtcactcgcgctagggcgtggcgtaggcaaaacgtcatgtacaagatcaccaatagtaaggctctggcggggtgccataggtggcgcagggacgaagctgttgcggtgtcctggtcgtctaacggtgcttcgcagtttgagggtctgcaaaactctcactctcgctgggggtcacctctggctgaattggaagtcatgggcgaacgccgcattgagctggctattgctactaagaatcacttggcggcgggtggcgcgctcatgatgtttgtgggcactgttcgacacaaccgctcacagtcatttgcgcaggttgaagcgggtattaagactgcgtactcttcgatggtgaaaacatctcagtggaagaaagaacgtgcacggtacggggtggagcacacctatagtgactatgaggtcacagactcttgggcgaacggttggcacttgcaccgcaacatgctgttgttcttggatcgtccactgtctgacgatgaactcaaggcgtttgaggattccatgttttcccgctggtctgctggtgtggttaaggccggtatggacgcgccactgcgtgagcacggggtcaaacttgatcaggtgtctacctggggtggagacgctgcgaaaatggcaacctacctcgctaagggcatgtctcaggaactgactggctccgctactaaaaccgcgtctaaggggtcgtacacgccgtttcagatgttggatatgttggccgatcaaagcgacgccggcgaggatatggacgctgttttggtggctcggtggcgtgagtatgaggttggttctaaaaacctgcgttcgtcctggtcacgtggggctaagcgtgctttgggcattgattacatagacgctgatgtacgtcgtgaaatggaagaagaactgtacaagctcgccggtctggaagcaccggaacgggtcgaatcaacccgcgttgctgttgctttggtgaagcccgatgattggaaactgattcagtctgatttcgcggttaggcagtacgttctcgattgcgtggataaggctaaggacgtggccgctgcgcaacgtgtcgctaatgaggtgctggcaagtctgggtgtggattccaccccgtgcatgatcgttatggatgatgtggacttggacgcggttctgcctactcatggggacgctactaagcgtgatctgaatgcggcggtgttcgcgggtaatgagcagactattcttcgcacccactaaaagcggcataaaccccgttcgatattttgtgcgatgaatttatggtcaatgtcgcgggggcaaactatgatgggtcttgttgttggcgtcccggaaaacgattccgaagcccaacctttcatagaaggcggcggtggaatcgaaatctcgtgatggcaggttgggcgtcgcttggtcggtcatttcgaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgtcgaagctcggcggatttgtcctactcaagctgatccgacaaaatccacacattatcccaggtgtccggatcggtcaaatacgctgccagctcatagaccgtatccaaagcatccggggctgatccccggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcgatggtgtcaacgtaaatgccgcttcgccttcgcgcgcgaattgcaagctgatccgggcttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaat

SEQ ID NO: 25 - DNA, 8842 bp, synthetic construct
  source 1..8842: mol_type = other DNA
gagctgttgacaattaatcatcggctcgtataatgtgtggaattgtgagcggataacaatttcacacaggaaacagaattaattaagctttggccactatttaaaaggaactcttatgaaagaatcgttctacactcacatcaatcaagaattagaaaatattaagaacttaggactttataagaaggaacggatcatcacttctccccaacaatctgaaattgcaatttccaatggtcagcatgtattaaatttttgcgcaaacaattacttaggattagcgaataaccagcaactaaaagaggctgcaaaagaaagtcttgataagtatggatttggcctttcctccgttcgctttatttgtggtacccaaaccattcataaagaattagaagacaaactatcctcttttttaggaatggaagatacaatcttgtactcttcttgctgggatgccaatggtggattatttgaaatccttttaggtcctgaagacgctgtaattagcgattctttaaatcacgccagtattattgatggtatccgtttatgtaaggcaaaacggttacgctacgaaaacaataatatgaaagatttagaagagaagctaaaggaagctgatggaacccgctttaaattagtagcgacagatggcgtattctcaatggatggtactattgccaatttacaaggcatttgtgatttagcagataaatataatgcgcttgtcatggtagacgattctcatgcggttggattcattggaaaaaaagggcgcggtactcatgaattttgcaatgtaatgggacgtgtagacattattacgggtactcttggaaaagcgttaggtggcgcctcaggcggttatacttctgctaaaaaagaagtcatcgatcttcttcgtcaacgttcacgcccttatcttttttcaaatactttagcgccatcaattgctggagcttctttacaagctttaaaactgattgaaagtagtgatgatttacgccaaaaactaattgaaaatagcgagtattttagagagaaaatgacaactcttgggtttactctccctcctggcgaacatcccattattccagtaatgctaggcgacgctaagctcgctcaagaatttgcagcaaaaatgctagaagagggaatttatgtaattggtttcttctatcctgtagtacctcaaggaaaagcccgtattcgcacgcagatgtctgctgttcattcgaaacaagatttagataaagccatcgctgcctttgaaaaagttggtaaaagtttacaagttatttaaaagaaaaaggaatgctatgaaagcattagttaaaaaatactctaagcctggactttggatggatgaagttccggtcccagaagttggagatgatgaagttttaattaaagtaaaaaaaacgtctatttgcggtacagatgttcatatttacaaatgggactcttgggcgcaaaaaacaattcctgttcctatggttgtcggtcacgaatttatgggaacaattgaagcgcttggcaaaaacgtaaagcatttacaagtaggtcaacgcgtcagcggcgaaggacacatcacttgtggacagtgccccccttgtaaaatgggaaataaacatttatgtatgaatacattgggtgttggtgttaaccgtcacggctgttttgctgaatactttaaccttccagctgaaaacgtctttcttttaccagatagcgtaaaagacaacgtagcttctatatttgatccttacggcaatgcagttcacacaacgttatcttttgaagtaactggtgaagatgttttgattacaggtgctggcccaatcggtatcatggcagcagctattgctcaaaaagctggagctcgtcacgtcgttattactgatgtaaatgaatatcgccttgatttagctaaaaaaatgggagctaccactaccgttaacgtggcaaaacaagacattaaagaagttatgaaagagcttgggattacgcacggatttactgtcggtcttgagatgtctggccacccaaacggcttaagcactcttttagaaacagctcagcatggcgcaaaaattgccttacttggcattttaccacctcacacaactgtagattgggatttagtcattttcaaaatgttaatggtcaaaggaatttatggacgagaagtctttcgtacctggtataaaatgaccaatcttttggaaagcggtcttaatttagaaccaatcattacccatcatttcccaattgatgattatgaaaagggatttgaagtcatgttatctggtaaatctggcaaagtcattcttgattggtaagtcgactctagaggatccccgggtaccgagctcgaattcagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttggggtgggcgaagaactccagcatgagatccccgcgctggaggatcatccagccattcggggtcgttcactggttcccctttctgatttctggcatagaagaacccccgtgaactgtgtggttccgggggttgctgatttttgcgagacttctcgcgcaattccctagcttaggtgaaaacaccatgaaacactagggaaacacccatgaaacacccattagggcagtagggcggcttcttcgtctagggcttgcatttgggcggtgatctggtctttagcgtgtgaaagtgtgtcgtaggtggcgtgctcaatgcactcgaacgtcacgtcatttaccgggtcacggtgggcaaagagaactagtgggttagacattgttttcctcgttgtcggtggtggtgagcttttctagccgctcggtaaacgcggcgatcatgaactcttggaggttttcaccgttctgcatgcctgcgcgcttcatgtcctcacgtagtgccaaaggaacgcgtgcggtgaccacgacgggcttagcctttgcctgcgcttctagtgcttcgatggtggcttgtgcctgcgcttgctgcgcctgtagtgcctgttgagcttcttgtagttgctgttctagctgtgccttggttgccatgctttaagactctagtagctttcctgcgatatgtcatgcgcatgcgtagcaaacattgtcctgcaactcattcattatgtgcagtgctcctgttactagtcgtacatactcatatttacctagtctgcatgcagtgcatgcacatgcagtcatgtcgtgctaatgtgtaaaacatgtacatgcagattgctgggggtgcagggggcggagccaccctgtccatgcggggtgtggggcttgccccgccggtacagacagtgagcaccggggcacctagtcgcggataccccccctaggtatcggacacgtaaccctcccatgtcgatgcaaatctttaacattgagtacgggtaagctggcacgcatagccaagctaggcggccaccaaacaccactaaaaattaatagtccctagacaagacaaacccccgtgcgagctaccaactcatatgcacgggggccacataacccgaaggggtttcaattgacaaccatagcactagctaagacaacgggcacaacacccgcacaaactcgcactgcgcaaccccgcacaacatcgggtctaggtaacactgagtaacactgaaatagaagtgaacacctctaaggaaccgcaggtcaatgagggttctaaggtcactcgcgctagggcgtggcgtaggcaaaacgtcatgtacaagatcaccaatagtaaggctctggcggggtgccataggtggcgcagggacgaagctgttgcggtgtcctggtcgtctaacggtgcttcgcagtttgagggtctgcaaaactctcactctcgctgggggtcacctctggctgaattggaagtcatgggcgaacgccgcattgagctggctattgctactaagaatcacttggcggcgggtggcgcgctcatgatgtttgtgggcactgttcgacacaaccgctcacagtcatttgcgcaggttgaagcgggtattaagactgcgtactcttcgatggtgaaaacatctcagtggaagaaagaacgtgcacggtacggggtggagcacacctatagtgactatgaggtcacagactcttgggcgaacggttggcacttgcaccgcaacatgctgttgttcttggatcgtccactgtctgacgatgaactcaaggcgtttgaggattccatgttttcccgctggtctgctggtgtggttaaggccggtatggacgcgccactgcgtgagcacggggtcaaacttgatcaggtgtctacctggggtggagacgctgcgaaaatggcaacctacctcgctaagggcatgtctcaggaactgactggctccgctactaaaaccgcgtctaaggggtcgtacacgccgtttcagatgttggatatgttggccgatcaaagcgacgccggcgaggatatggacgctgttttggtggctcggtggcgtgagtatgaggttggttctaaaaacctgcgttcgtcctggtcacgtggggctaagcgtgctttgggcattgattacatagacgctgatgtacgtcgtgaaatggaagaagaactgtacaagctcgccggtctggaagcaccggaacgggtcgaatcaacccgcgttgctgttgctttggtgaagcccgatgattggaaactgattcagtctgatttcgcggttaggcagtacgttctcgattgcgtggataaggctaaggacgtggccgctgcgcaacgtgtcgctaatgaggtgctggcaagtctgggtgtggattccaccccgtgcatgatcgttatggatgatgtggacttggacgcggttctgcctactcatggggacgctactaagcgtgatctgaatgcggcggtgttcgcgggtaatgagcagactattcttcgcacccactaaaagcggcataaaccccgttcgatattttgtgcgatgaatttatggtcaatgtcgcgggggcaaactatgatgggtcttgttgttggcgtcccggaaaacgattccgaagcccaacctttcatagaaggcggcggtggaatcgaaatctcgtgatggcaggttgggcgtcgcttggtcggtcatttcgaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgtcgaagctcggcggatttgtcctactcaagctgatccgacaaaatccacacattatcccaggtgtccggatcggtcaaatacgctgccagctcatagaccgtatccaaagcatccggggctgatccccggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcgatggtgtcaacgtaaatgccgcttcgccttcgcgcgcgaattgcaagctgatccgggcttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaat

SEQ ID NO: 26 - DNA, 8960 bp, synthetic construct
  source 1..8960: mol_type = other DNA
gagctgttgacaattaatcatcggctcgtataatgtgtggaattgtgagcggataacaatttcacacaggaaacagaattaattaagcttaaaccccatgaaagaaataccgcccatgcccgcaccgagttcgacccttgccaaaaccctgccgcagaccatgcaggcgatccaaaaacccgctgcagcctctggctttgcgctcatcgaagctgaacgccctgagccgggtccggatgatgtgctgattcaagtgtctctggcatccatctgcgggactgatttacacatcacccagtgggacgagtggagtgcttctcggattaagccccccctgatttacggacacgagttttgcggtgtggtcgcagccattggccgtaatgtggacaacgtcaaagtgggcgactttgtctctgcggaaatgcacctgccgtgccgcacatgccggcaatgtatgaccggaaatttccatatttgcgaaaaagtggaaattgtgggcatcgaccgcaacggctgctttgccaattatgtagtcattccgcgctggcaaatcatcaaactccccgcttcaatcacacccgaggtaggggcctgtttagactctttgggtaatgcggttcatgccgtctccaaaggcaatgtttccggaaagaccgtctatgttgttggctgcggacccattggcttgttttccatccgtgtgagtaaagcgctgggcgctacccgggtgtttgcctcggatatttctccgtaccgcctagaaatggcgaagacggctggcgctacacacgtccttgatgcccgcgaatgcaacccttctgactggatgctagaggccacgcaaggtcgcggggtcgatgtggtgctggaaatgtccgggaatgagcgggcgatcaacgatgcctttcaaggtctggccttgggcggaacgatggtactgctcggcattccacaaggcaatgtttcgctgaatttaaaccgcgatgtgatctttaaagaagccactgtccttggggttaacggtcgagagatgtttcaaacttggttcttgatgttagaactccttgaatcgggacgactgaatattgattttattatgacccaccgctttgcgttgagtgaatttgagacggccattgagctgatcgcctctggcaaaagcggcaaaatcttgctcgaaccttctttttaaatcccttcttttaaaacgacacacgaaggacactgcccgccatgacaagcacaaatcaagcagaaacccctcaggcattaaaaagccaccgtctggactttctggaagcagagtgccaggaacttaaagagcaacatctttttcagtcgcttcgggtaatgagttcagcacaaggggccaaaattcagctagacggcaaatcggtcattaatttatcttccaataactatctgggtctcaacacccaccccaaactgattaccgcggccaaaaaagccatcgatgactacggcgtgggcaccggttctgttcgcaccattattggcaccatggatatccaccaggcctttgaaaaacgactcgcccagtttaagcatactgaggcaacgcttgtgcttcaatctggctttacggccaacaatgcggctgttacctccattttgggcgaaggggatgttgtgatcagcgacagcctgaatcatgccagcatcattgatgcctgccgtctgctgaagaaggttcctcgttatgtctatgcccatgcggatatgactgagctggaagcacggctaaaagaaacacaatctgcccgtcgccggctggtggtgacagatggggtgttttcgatggacggagacattgccccgctaccagagattgtggcgctgtgtgaacagtacaacgcgattgtgatggtggatgacgcccacgccagtggggtattcggtaaaaacggtcgcggtactgtcgatcattttaatctccacggccgtgtggatatccaaattggcacgctatccaaagccattggcgccctgggcggttacgtggcatgtacgcaagccatgcgcgatatcatgatcaaccgggctaggccctttttgttcagcacctctgcccccccttcggtcgtgatgacctgtatggcagcacttgatctgcttgaaactgacacggcgctgatcgatcagctttggtacaacacccgctatttcaaggctgggctcaagtccatcggttacaacgttcaaagcgattcacccattatcccggtgattgcaggcaccagtgaaaaagcccaccgtctcagtgagcggctctttgaagaaggggtctatgttcgatcgattgtttaccccaccgttgctcttgataaggcccgggttcgagtgattttaagcgccacccacacgcaaacacagctcgatcaggccatcgctgcctttgaaaaagtcggaaaagagttggatctgattggcccataagtcgactctagaggatccccgggtaccgagctcgaattcagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttggggtgggcgaagaactccagcatgagatccccgcgctggaggatcatccagccattcggggtcgttcactggttcccctttctgatttctggcatagaagaacccccgtgaactgtgtggttccgggggttgctgatttttgcgagacttctcgcgcaattccctagcttaggtgaaaacaccatgaaacactagggaaacacccatgaaacacccattagggcagtagggcggcttcttcgtctagggcttgcatttgggcggtgatctggtctttagcgtgtgaaagtgtgtcgtaggtggcgtgctcaatgcactcgaacgtcacgtcatttaccgggtcacggtgggcaaagagaactagtgggttagacattgttttcctcgttgtcggtggtggtgagcttttctagccgctcggtaaacgcggcgatcatgaactcttggaggttttcaccgttctgcatgcctgcgcgcttcatgtcctcacgtagtgccaaaggaacgcgtgcggtgaccacgacgggcttagcctttgcctgcgcttctagtgcttcgatggtggcttgtgcctgcgcttgctgcgcctgtagtgcctgttgagcttcttgtagttgctgttctagctgtgccttggttgccatgctttaagactctagtagctttcctgcgatatgtcatgcgcatgcgtagcaaacattgtcctgcaactcattcattatgtgcagtgctcctgttactagtcgtacatactcatatttacctagtctgcatgcagtgcatgcacatgcagtcatgtcgtgctaatgtgtaaaacatgtacatgcagattgctgggggtgcagggggcggagccaccctgtccatgcggggtgtggggcttgccccgccggtacagacagtgagcaccggggcacctagtcgcggataccccccctaggtatcggacacgtaaccctcccatgtcgatgcaaatctttaacattgagtacgggtaagctggcacgcatagccaagctaggcggccaccaaacaccactaaaaattaatagtccctagacaagacaaacccccgtgcgagctaccaactcatatgcacgggggccacataacccgaaggggtttcaattgacaaccatagcactagctaagacaacgggcacaacacccgcacaaactcgcactgcgcaaccccgcacaacatcgggtctaggtaacactgagtaacactgaaatagaagtgaacacctctaaggaaccgcaggtcaatgagggttctaaggtcactcgcgctagggcgtggcgtaggcaaaacgtcatgtacaagatcaccaatagtaaggctctggcggggtgccataggtggcgcagggacgaagctgttgcggtgtcctggtcgtctaacggtgcttcgcagtttgagggtctgcaaaactctcactctcgctgggggtcacctctggctgaattggaagtcatgggcgaacgccgcattgagctggctattgctactaagaatcacttggcggcgggtggcgcgctcatgatgtttgtgggcactgttcgacacaaccgctcacagtcatttgcgcaggttgaagcgggtattaagactgcgtactcttcgatggtgaaaacatctcagtggaagaaagaacgtgcacggtacggggtggagcacacctatagtgactatgaggtcacagactcttgggcgaacggttggcacttgcaccgcaacatgctgttgttcttggatcgtccactgtctgacgatgaactcaaggcgtttgaggattccatgttttcccgctggtctgctggtgtggttaaggccggtatggacgcgccactgcgtgagcacggggtcaaacttgatcaggtgtctacctggggtggagacgctgcgaaaatggcaacctacctcgctaagggcatgtctcaggaactgactggctccgctactaaaaccgcgtctaaggggtcgtacacgccgtttcagatgttggatatgttggccgatcaaagcgacgccggcgaggatatggacgctgttttggtggctcggtggcgtgagtatgaggttggttctaaaaacctgcgttcgtcctggtcacgtggggctaagcgtgctttgggcattgattacatagacgctgatgtacgtcgtgaaatggaagaagaactgtacaagctcgccggtctggaagcaccggaacgggtcgaatcaacccgcgttgctgttgctttggtgaagcccgatgattggaaactgattcagtctgatttcgcggttaggcagtacgttctcgattgcgtggataaggctaaggacgtggccgctgcgcaacgtgtcgctaatgaggtgctggcaagtctgggtgtggattccaccccgtgcatgatcgttatggatgatgtggacttggacgcggttctgcctactcatggggacgctactaagcgtgatctgaatgcggcggtgttcgcgggtaatgagcagactattcttcgcacccactaaaagcggcataaaccccgttcgatattttgtgcgatgaatttatggtcaatgtcgcgggggcaaactatgatgggtcttgttgttggcgtcccggaaaacgattccgaagcccaacctttcatagaaggcggcggtggaatcgaaatctcgtgatggcaggttgggcgtcgcttggtcggtcatttcgaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgtcgaagctcggcggatttgtcctactcaagctgatccgacaaaatccacacattatcccaggtgtccggatcggtcaaatacgctgccagctcatagaccgtatccaaagcatccggggctgatccccggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcgatggtgtcaacgtaaatgccgcttcgccttcgcgcgcgaattgcaagctgatccgggcttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaat

SEQ ID NO: 27 - DNA, 8847 bp, synthetic construct
  source 1..8847: mol_type = other DNA
gagctgttgacaattaatcatcggctcgtataatgtgtggaattgtgagcggataacaatttcacacaggaaacagaattaattaagcttaatttgccctgtctggagaatcgcaatgcgtggagaattttatcagcagttaaccaacgatctggaaaccgcacgggcggaagggttgtttaaagaagagcgcattattacgtctgcgcagcaagcagatatcactgtggctgatggaagccacgtcattaacttttgtgccaacaactatctcgggctggcgaatcatcctgatctgattgcggcggcaaaggcgggaatggattctcacggtttcggcatggcttcggtgcgttttatttgcggcactcaggacagccataaagagcttgaacaaaaactggcggccttcctggggatggaagatgcgattctctactcttcctgctttgatgctaacggtggcctgtttgaaacgcttctgggtgcggaagacgccattatctccgacgcactgaaccacgcgtctattattgatggtgtgcgtctgtgcaaagctaaacgctatcgctatgccaacaacgatatgcaggagctggaagcacgtctgaaagaagcgcgtgaagccggtgcgcgtcatgtgctgatcgccaccgatggtgtgttctcaatggacggcgtgattgccaacctgaagggtgtttgcgatctggcagataaatatgatgccctggtgatggtagacgactcccacgcggtcggttttgtcggtgaaaatggtcgtggttcccatgaatactgcgatgtgatgggccgggtcgatattatcaccggtacgcttggtaaagcgctgggcggggcttctggtggttataccgcggcgcgcaaagaagtggttgagtggctgcgccagcgttctcgtccgtacctgttctccaactcgctggcaccggccattgttgccgcgtccatcaaagtactggagatggtcgaagcgggcagcgaactgcgtgaccgtctgtgggcgaacgcgcgtcagttccgtgagcaaatgtcggcggcgggctttaccctggcgggagccgatcacgccattattccggtcatgcttggtgatgcggtagtggcgcagaaatttgcccgtgagctgcaaaaagagggcatttacgttaccggtttcttctatccggtcgttccgaaaggtcaggcgcgtattcgtacccagatgtctgcggcgcatacccctgagcaaattacgcgtgcagtagaagcatttacgcgtattggtaaacaactgggcgttatcgcctgaggatgtgagatgaaagcgttatccaaactgaaagcggaagagggcatctggatgaccgacgttcctgtaccggaactcgggcataacgatctgctgattaaaatccgtaaaacagccatctgcgggactgacgttcacatctataactgggatgagtggtcgcaaaaaaccatcccggtgccgatggtcgtgggccatgaatatgtcggtgaagtggtaggtattggtcaggaagtgaaaggcttcaagatcggcgatcgcgtttctggcgaaggccatatcacctgtggtcattgccgcaactgtcgtggtggtcgtacccatttgtgccgcaacacgattggcgttggtgttaatcgcccgggctgctttgccgaatatctggtgatcccggcattcaacgccttcaaaatccccgacaatatttccgatgacttagccgcaatttttgatcccttcggtaacgccgtgcataccgcgctgtcgtttgatctggtgggcgaagatgtgctggtttctggtgcaggcccgattggtattatggcagcggcggtggcgaaacacgttggtgcacgcaatgtggtgatcactgatgttaacgaataccgccttgagctggcgcgtaaaatgggtatcacccgtgcggttaacgtcgccaaagaaaatctcaatgacgtgatggcggagttaggcatgaccgaaggttttgatgtcggtctggaaatgtccggtgcgccgccagcgtttcgtaccatgcttgacaccatgaatcacggcggccgtattgcgatgctgggtattccgccgtctgatatgtctatcgactggaccaaagtgatctttaaaggcttgttcattaaaggtatttacggtcgtgagatgtttgaaacctggtacaagatggcggcgctgattcagtctggcctcgatctttcgccgatcattacccatcgtttctctatcgatgatttccagaagggctttgacgctatgcgttcgggccagtccgggaaagttattctgagctgggattaagtcgactctagaggatccccgggtaccgagctcgaattcagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttggggtgggcgaagaactccagcatgagatccccgcgctggaggatcatccagccattcggggtcgttcactggttcccctttctgatttctggcatagaagaacccccgtgaactgtgtggttccgggggttgctgatttttgcgagacttctcgcgcaattccctagcttaggtgaaaacaccatgaaacactagggaaacacccatgaaacacccattagggcagtagggcggcttcttcgtctagggcttgcatttgggcggtgatctggtctttagcgtgtgaaagtgtgtcgtaggtggcgtgctcaatgcactcgaacgtcacgtcatttaccgggtcacggtgggcaaagagaactagtgggttagacattgttttcctcgttgtcggtggtggtgagcttttctagccgctcggtaaacgcggcgatcatgaactcttggaggttttcaccgttctgcatgcctgcgcgcttcatgtcctcacgtagtgccaaaggaacgcgtgcggtgaccacgacgggcttagcctttgcctgcgcttctagtgcttcgatggtggcttgtgcctgcgcttgctgcgcctgtagtgcctgttgagcttcttgtagttgctgttctagctgtgccttggttgccatgctttaagactctagtagctttcctgcgatatgtcatgcgcatgcgtagcaaacattgtcctgcaactcattcattatgtgcagtgctcctgttactagtcgtacatactcatatttacctagtctgcatgcagtgcatgcacatgcagtcatgtcgtgctaatgtgtaaaacatgtacatgcagattgctgggggtgcagggggcggagccaccctgtccatgcggggtgtggggcttgccccgccggtacagacagtgagcaccggggcacctagtcgcggataccccccctaggtatcggacacgtaaccctcccatgtcgatgcaaatctttaacattgagtacgggtaagctggcacgcatagccaagctaggcggccaccaaacaccactaaaaattaatagtccctagacaagacaaacccccgtgcgagctaccaactcatatgcacgggggccacataacccgaaggggtttcaattgacaaccatagcactagctaagacaacgggcacaacacccgcacaaactcgcactgcgcaaccccgcacaacatcgggtctaggtaacactgagtaacactgaaatagaagtgaacacctctaaggaaccgcaggtcaatgagggttctaaggtcactcgcgctagggcgtggcgtaggcaaaacgtcatgtacaagatcaccaatagtaaggctctggcggggtgccataggtggcgcagggacgaagctgttgcggtgtcctggtcgtctaacggtgcttcgcagtttgagggtctgcaaaactctcactctcgctgggggtcacctctggctgaattggaagtcatgggcgaacgccgcattgagctggctattgctactaagaatcacttggcggcgggtggcgcgctcatgatgtttgtgggcactgttcgacacaaccgctcacagtcatttgcgcaggttgaagcgggtattaagactgcgtactcttcgatggtgaaaacatctcagtggaagaaagaacgtgcacggtacggggtggagcacacctatagtgactatgaggtcacagactcttgggcgaacggttggcacttgcaccgcaacatgctgttgttcttggatcgtccactgtctgacgatgaactcaaggcgtttgaggattccatgttttcccgctggtctgctggtgtggttaaggccggtatggacgcgccactgcgtgagcacggggtcaaacttgatcaggtgtctacctggggtggagacgctgcgaaaatggcaacctacctcgctaagggcatgtctcaggaactgactggctccgctactaaaaccgcgtctaaggggtcgtacacgccgtttcagatgttggatatgttggccgatcaaagcgacgccggcgaggatatggacgctgttttggtggctcggtggcgtgagtatgaggttggttctaaaaacctgcgttcgtcctggtcacgtggggctaagcgtgctttgggcattgattacatagacgctgatgtacgtcgtgaaatggaagaagaactgtacaagctcgccggtctggaagcaccggaacgggtcgaatcaacccgcgttgctgttgctttggtgaagcccgatgattggaaactgattcagtctgatttcgcggttaggcagtacgttctcgattgcgtggataaggctaaggacgtggccgctgcgcaacgtgtcgctaatgaggtgctggcaagtctgggtgtggattccaccccgtgcatgatcgttatggatgatgtggacttggacgcggttctgcctactcatggggacgctactaagcgtgatctgaatgcggcggtgttcgcgggtaatgagcagactattcttcgcacccactaaaagcggcataaaccccgttcgatattttgtgcgatgaatttatggtcaatgtcgcgggggcaaactatgatgggtcttgttgttggcgtcccggaaaacgattccgaagcccaacctttcatagaaggcggcggtggaatcgaaatctcgtgatggcaggttgggcgtcgcttggtcggtcatttcgaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgtcgaagctcggcggatttgtcctactcaagctgatccgacaaaatccacacattatcccaggtgtccggatcggtcaaatacgctgccagctcatagaccgtatccaaagcatccggggctgatccccggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcgatggtgtcaacgtaaatgccgcttcgccttcgcgcgcgaattgcaagctgatccgggcttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaat

SEQ ID NO: 28 - DNA, 44 bp, synthetic construct
  source 1..44: mol_type = other DNA
tcgagctcggtacccggggatcctctggctgatgatgatctcac

SEQ ID NO: 29 - DNA, 45 bp, synthetic construct
  source 1..45: mol_type = other DNA
ctagggatagtagacacttcacaagtgtcttacctcggctggttg

SEQ ID NO: 30 - DNA, 47 bp, synthetic construct
  source 1..47: mol_type = other DNA
cttgtgaagtgtctactatccctagaattactcagcgggcgcaccac

SEQ ID NO: 31 - DNA, 46 bp, synthetic construct
  source 1..46: mol_type = other DNA
agcttgcatgcctgcaggtcgactctgcacgacggttcagctcttc

SEQ ID NO: 32 - DNA, 11554 bp, synthetic construct
  source 1..11554: mol_type = other DNA
cgataagctagcttcacgctgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgctagaggatcgatcctttttaacccatcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaaaagtaaatcgcgcgggtttgttactgataaagcaggcaagacctaaaatgtgtaaagggcaaagtgtatactttggcgtcaccccttacatattttaggtctttttttattgtgcgtaactaacttgccatcttcaaacaggagggctggaagaagcagaccgctaacacagtacataaaaaaggagacatgaacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagtttctgaatttgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaancgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccgagcttcctgctgaacatcaaaggcaagaaaacatctgttgtcaaagacagcatccttgaacaaggacaattaacagttaacaaataaaaacgcaaaagaaaatgccgatgggtaccgagcgaaatgaccgaccaagcgacgcccaacctgccatcacgagatttcgattccaccgccgccttctatgaaaggttgggcttcggaatcgttttccgggacgccctcgcggacgtgctcatagtccacgacgcccgtgattttgtagccctggccgacggccagcaggtaggccgacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgccccgctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgctgcttccctgctgttttgtggaatatctaccgactggaaacaggcaaatgcaggaaattactgaactgaggggacaggcgagagacgatgccaaagagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagggctggatgatcctccagcgcggggatctcatgctggagttcttcgcccaccccaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattgcgtggcaatcaggctttacctcagcgaagaattcgacgacgccacccgcgtctacaccgtccgatcctttgatgccgcaactgaaagcatcgtggtggatgtggttcaacaccaccacgaaagccccatgatgcgctggtcagacaccgtaaaaatcaacgacaccctcgtgctcaccggaccccgcccacactttgtcatccccgaaggcgaacaagcagcactcttccttgatgacaccgccatccccgctctcgccgctattttggatcaatggccaacagatcttcgtggcaaaggatgggttgtcactgacgatcccgcagccttcgatgaactacccagcatcgacggactggaactgaacctgctcgcgccgggatcagatccaactgttcagccacttgcccaacaggcatatgacctggaaaacccagaaacttacgtggtgtgggcagccggcgagcgagatgaaataaaatccatccgcaggcacttccgcaagcaggtgggattggaaaaagatgcagtggccgtgtttgggtactggaaatacaacaccaccaacactcagatcgatgcagtccgcaaagaaaactacatgaagatgctctctgaagggctacagctggaaaacttcgacgacctctcattggagatttaaggggtcgagtttttagaatgggtgtttgcattgattggcgtctgatgagctcttagaagcgattctgggagggcatgatttttgggattcggagctgaatgtggactgattttgggggaaccagactggcgtgcccaaatgtcgtgtcttcagtcctaggtcaatattacaggccataaactaatgcttatcgacgtacccccgcacgtgcccactccgcgtgagcggtacttgcggcatcgacacgtgcgcgttgctccgcaacccgcacgcccgcggttccaccgcgcgttgcacgggaagccactgcaccatcaatggtgagcacttcccgtacctctggggtcagacgtgcatcaacgccactgagttcttcatcagtgagatcgataaggtccactcccctggactccgcgatccgcacgcaagcgccggatgcttcgtgtgcctcacggaatggaacgccctggcgcaccatccactcagccaagtcggtggcaagggtgaaacctgctggtgcaagttcacgcatgcgctcggtgttgaaggtcaaggtggaaaccaaaccagtcattgcagggagcagcaggttgagctgcgccacggaatctacgattggttccttatcttcctgcaggtcgcggttgtacgctaaaggctgtgccttcagggtagccagcagaccggtgaggttaccgatcaagcgaccagacttgccacgggtcagctctgccacgtcagggttcttcttctgcggcatgattgagctgcctgtggaccaggaatcagacaaggtgatgtaaccaaattctggggtgcaccatgcgatgatttcttcagccaagcgggacatatccactgcaagctgcgccagcacgaaggcggtttcagatgcgaaatcgcgggagctggtggcatcaatggagttatctgctgcggaatcaaagccgagttcttcagcgattgcttcagggttgagcttcaaagaggaaccagcaagtgcgccggaaccgtaaggagacaccgcaagacgcttgtccaggtcacggatacgatcaatatcgcgcagcaaaggctgtgcgtgtgccagcagctggtgtgccagaaggaccggctgagctgcctggaagtgggtcttgcctggcatgatcgcgcctgcatgtgccttagcttgggcgctgagggcgtcgacaagctcggttgttcccagcgcgatgtcgcgcactgcgtcgcggacccacatgcggaacagggttgccacctggtcgttgcgggaacgaccagcgcgcagacggccgcccacctcaggaccaacgcggtcaatcagaccgcgttccatcgcgccgtgcacatcctcatcagaaggcagcggaccgaaggttccgtcggcgacatccttgcccagctgatcaagcccagccagcatggtggctagatcttcatcagaaagtagatctgcttggtgcaaaaccttggcgtgtgccttggaggccaacacatcataaggggccaaaacccagtcgaaatgagtggagacactcaaggcgaacatggcctcggagggtccaccggagaagcggccgccccacagcgcaccttcattggttccgtgctgttccatgtggtgttcttctactttctaaatacttgaagttctagtctggatgcttgaaaaggtggcttagttgttgccagcttcgcgatcgcgcttgttagcgatcttggaggacagaccgtgcagctggacaaagcccttagccagggtctggtcgaaggtgtcgccggtgtcgtaggtagccaggttgaagtcgtacagggagtggctggaacgacgaccattgatggtgatggaacctgcgtgcagaaccatgcggatatcgccggtgacgtgctcctgggtggaatcaatgaacgcgtccagggagcgcttcagaggtccgaaccacaggccgtcgtatacttcctcagcccaacgtgcgtcaacgccgcgcttgtagcgagccagttcgcgctcgatggtgacatcttccaaagcctcgtgagccttaatcagtgcgattgcgcctggtgcttcgtagatttcgcgggacttgatgcccacgagacggtcctcaaccatgtcaaggcggccaacgccctgtgcgcctgcacgacggttcagctcttcaatagcctgcagtacggagactggacggccatcgatggagactggcttgccaccctcgaaggagatgatgacctcatctggagcgttacccagagctggatcctcggtgtatgcgtagatgtccttggttggagcattccacagatcttccaggtaaccggtctcaatagcgcggccccagacgttctggtcgatggagaatggggacttcacggactgctcgattggaacgttgttctcctcggcgaaggcgatagccttgtcgcgggtccatgcgaagtcacgagcaggtgcaatgatctccaggtttggatcggtgtccatgaagccgacctcgaaacgaacctggtcgttgcccttaccagtgcagccgtgtgcaacgtgggtaccgttgaactgcttgccagcctcaacgaggtgcttgacgatcagtgggcgggagattgcagaaaccagtgggtactgcttcatgtacatgccgtttgccttgatggttggcaggcagtactcctcagcgaactcatcctttgcatcaacaacgatggactcagctgcaccggcatccaatgcacgctggcgaacgttgtccatgttctctccaccctggcccaggtcgagggaaactgcgatgacttcaccatcaatcatcttcttcaggtatggaattgccacagtggtgtccagaccgccggagtatgcaagaacgatgcggttagtcatgaggtgtgctccttcattttaagttccttgccagccgtgttattcttacctcggctggttggccagcagccacaccagcagtgctttctgagcgtggaggcggttttctgcttcatcgaaaactttggacgctggtccatcaatcacggaggctgccacttctttgccacggtaggcaggaaggcagtgcaggaagatggcgccgtcgttagctttcgccatgacctcatcgttgacctggtaaggaacgaaaggtgtggtgcgatcgatgccgtcgttttccatacccatggatacccaggtatcggtgatgacaacatcggcgccggcaacctcgtcgaggctgtcggtgacaacaaccttcgcgccggtttcctggccacgcttttccgcgcgctccacgaattccgcacgaggctggaacccttcaggagcgatgatggaaatatccatgcccgcggtggcaaagccaatcatgtaggagttggccatgttgttgtcgccatcgcccaggtacacagccttcttacccttaaggcctgctgggccttcttcagggctgaggttttccacgatagtctgcagatcagccagaatctggcatgggtgcagatcatcggacaaggagttcaccagcggcacagtggacgtctccgccatggcgtggaaattgctgtgtgcgtaggtgcgccacacaattgcttccacgtagcgggacaatacagctgcggtgtcctgcagggactcgcccttacccatctgtgagctaccggaatccacgacgatggcgtgtccacccaaatgagcgatgcccgcgtcgaaggagaagcgagtacgagttgaagtcttatcaaaaagaactgcaacggactttggtccctcgagtggacgctccgaaaacggcgctgccttgagctttgcggctagggtcaaaacctctgcctgctctgcaggggtgagatcatcatcagccagaaaatggcgaacctgtggttgtgaagtcataagtttgagtcctttatgcgattgtctcggcagttcgattgtaggaaatgcaatgtgtcaagcagttaggtcaatcagagcatcggtcactcaagaattcttgagctgacagaacccaagggcgagtatggcgaattttttcaactcacgcaccaaacctactcacattggataattcgtcgagtcaactccgtgtcaacctgtcctcaactttagcggggaaacaaagatttcgcagcagatgcaaaatcatacagacacattcaaaaatggacattgaccccaaacgggggcatttccaacacttgcgctaaatagtttttgcagggaacgaacctgaaatcgttcgcactacgatctgctcccccgaatggggttaattatgaactacattacacccccgtgaatccacaaattaaagtctcagatttgaagccaatgtgatgcaactcacactggcttcaatggtaatcccaataggctcggtcgtttttaggcttcaatgcatgcctgtctggtttctcggctctcaaaccgaacaggagtgcccacggctggcaaaaagttcagttcttgggcacgctagtctggttttacctttttgagtgccccgaactgccaaacgagagtgcccatgaggggctaaatctggatccgcccgcacaaaacaactgccgcagcacaacgaatcaacgctgtgctgcggcagtcacttttttgaaggattaacgccaacccatggtcatgagtaggccgatgatcatcagaccgaagccgatgccatagttccatgcaccaagatcagccatgaatgggatctgtgggcccacgaggtagttaatgatcaaccaggctaggccgacgatcatgaaggcaaacatgatgaccttgtaccacattggggttccggcggaattgatcttaaccggggtgcggtttgcgcttgggttgcttgaaaccggtgcggtctcgtttttagttactcttgcctttggcattgttcacaccttcataatttattgtcttggaaaaaccccagcaactggtacttatcttgtttgagcttaacgtatcagttgccgggccagctaccttgacggtttccttgttggctattgcacgagtgcagcgagatcgaattcgaagagtcgcacttgcacttgggcgtctttgcggaagagggttgcaggggttggggattggaatccgattttgttttgatccacgagggctgcggtgtggatggggtcgccgacgatcagggattgatctggggcggtccagccagcagcgcggagggcactgatggcctgttcggtgaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcg

SEQ ID NO: 33 - DNA, 6026 bp, synthetic construct
  source 1..6026: mol_type = other DNA
ttccggctcgtatgttgtgtggaattgtgagcggataacaatttcacacaggaaacagctatgaccatgattacgaattgcgtggcaatcaggctttacctcagcgaagaattcgacgacgccacccgcgtctacaccgtccgatcctttgatgccgcaactgaaagcatcgtggtggatgtggttcaacaccaccacgaaagccccatgatgcgctggtcagacaccgtaaaaatcaacgacaccctcgtgctcaccggaccccgcccacactttgtcatccccgaaggcgaacaagcagcactcttccttgatgacaccgccatccccgctctcgccgctattttggatcaatggccaacagatcttcgtggcaaaggatgggttgtcactgacgatcccgcagccttcgatgaactacccagcatcgacggactggaactgaacctgctcgcgccgggatcagatccaactgttcagccacttgcccaacaggcatatgacctggaaaacccagaaacttacgtggtgtgggcagccggcgagcgagatgaaataaaatccatccgcaggcacttccgcaagcaggtgggattggaaaaagatgcagtggccgtgtttgggtactggaaatacaacaccaccaacactcagatcgatgcagtccgcaaagaaaactacatgaagatgctctctgaagggctacagctggaaaacttcgacgacctctcattggagatttaaggggtcgagtttttagaatgggtgtttgcattgattggcgtctgatgagctcttagaagcgattctgggagggcatgatttttgggattcggagctgaatgtggactgattttgggggaaccagactggcgtgcccaaatgtcgtgtcttcagtcctaggtcaatattacaggccataaactaatgcttatcgacgtacccccgcacgtgcccactccgcgtgagcggtacttgcggcatcgacacgtgcgcgttgctccgcaacccgcacgcccgcggttccaccgcgcgttgcacgggaagccactgcaccatcaatggtgagcacttcccgtacctctggggtcagacgtgcatcaacgccactgagttcttcatcagtgagatcgataaggtccactcccctggactccgcgatccgcacgcaagcgccggatgcttcgtgtgcctcacggaatggaacgccctggcgcaccatccactcagccaagtcggtggcaagggtgaaacctgctggtgcaagttcacgcatgcgctcggtgttgaaggtcaaggtggaaaccaaaccagtcattgcagggagcagcaggttgagctgcgccacggaatctacgattggttccttatcttcctgcaggtcgcggttgtacgctaaaggctgtgccttcagggtagccagcagaccggtgaggttaccgatcaagcgaccagacttgccacgggtcagctctgccacgtcagggttcttcttctgcggcatgattgagctgcctgtggaccaggaatcagacaaggtgatgtaaccaaattctggggtgcaccatgcgatgatttcttcagccaagcgggacatatccactgcaagctgcgccagcacgaaggcggtttcagatgcgaaatcgcgggagctggtggcatcaatggagttatctgctgcggaatcaaagccgagttcttcagcgattgcttcagggttgagcttcaaagaggaaccagcaagtgcgccggaaccgtaaggagacaccgcaagacgcttgtccaggtcacggatacgatcaatatcgcgcagcaaaggctgtgcgtgtgccagcagctggtgtgccagaaggaccggctgagctgcctggaagtgggtcttgcctggcatgatcgcgcctgcatgtgccttagcttgggcgctgagggcgtcgacaagctcggttgttcccagcgcgatgtcgcgcactgcgtcgcggacccacatgcggaacagggttgccacctggtcgttgcgggaacgaccagcgcgcagacggccgcccacctcaggaccaacgcggtcaatcagaccgcgttccatcgcgccgtgcacatcctcatcagaaggcagcggaccgaaggttccgtcggcgacatccttgcccagctgatcaagcccagccagcatggtggctagatcttcatcagaaagtagatctgcttggtgcaaaaccttggcgtgtgccttggaggccaacacatcataaggggccaaaacccagtcgaaatgagtggagacactcaaggcgaacatggcctcggagggtccaccggagaagcggccgccccacagcgcaccttcattggttccgtgctgttccatgtggtgttcttctactttctaaatacttgaagttctagtctggatgcttgaaaaggtggcttagttgttgccagcttcgcgatcgcgcttgttagcgatcttggaggacagaccgtgcagctggacaaagcccttagccagggtctggtcgaaggtgtcgccggtgtcgtaggtagccaggttgaagtcgtacagggagtggctggaacgacgaccattgatggtgatggaacctgcgtgcagaaccatgcggatatcgccggtgacgtgctcctgggtggaatcaatgaacgcgtccagggagcgcttcagaggtccgaaccacaggccgtcgtatacttcctcagcccaacgtgcgtcaacgccgcgcttgtagcgagccagttcgcgctcgatggtgacatcttccaaagcctcgtgagccttaatcagtgcgattgcgcctggtgcttcgtagatttcgcgggacttgatgcccacgagacggtcctcaaccatgtcaaggcggccaacgccctgtgcgcctgcacgacggttcagctcttcaatagcctgcagtacggagactggacggccatcgatggagactggcttgccaccctcgaaggagatgatgacctcatctggagcgttacccagagctggatcctcggtgtatgcgtagatgtccttggttggagcattccacagatcttccaggtaaccggtctcaatagcgcggccccagacgttctggtcgatggagaatggggacttcacggactgctcgattggaacgttgttctcctcggcgaaggcgatagccttgtcgcgggtccatgcgaagtcacgagcaggtgcaatgatctccaggtttggatcggtgtccatgaagccgacctcgaaacgaacctggtcgttgcccttaccagtgcagccgtgtgcaacgtgggtaccgttgaactgcttgccagcctcaacgaggtgcttgacgatcagtgggcgggagattgcagaaaccagtgggtactgcttcatgtacatgccgtttgccttgatggttggcaggcagtactcctcagcgaactcatcctttgcatcaacaacgatggactcagctgcaccggcatccaatgcacgctggcgaacgttgtccatgttctctccaccctggcccaggtcgagggaaactgcgatgacttcaccatcaatcatcttcttcaggtatggaattgccacagtggtgtccagaccgccggagtatgcaagaacgatgcggttagtcatgaggtgtgctccttcattttaagttccttgccagccgtgttattcttacctcggctggttggccagcagccacaccagcagtgctttctgagcgtggaggcggttttctgcttcatcgaaaactttggacgctggtccatcaatcacggaggctgccacttctttgccacggtaggcaggaaggcagtgcaggaagatggcgccgtcgttagctttcgccatgacctcatcgttgacctggtaaggaacgaaaggtgtggtgcgatcgatgccgtcgttttccatacccatggatacccaggtatcggtgatgacaacatcggcgccggcaacctcgtcgaggctgtcggtgacaacaaccttcgcgccggtttcctggccacgcttttccgcgcgctccacgaattccgcacgaggctggaacccttcaggagcgatgatggaaatatccatgcccgcggtggcaaagccaatcatgtaggagttggccatgttgttgtcgccatcgcccaggtacacagccttcttacccttaaggcctgctgggccttcttcagggctgaggttttccacgatagtctgcagatcagccagaatctggcatgggtgcagatcatcggacaaggagttcaccagcggcacagtggacgtctccgccatggcgtggaaattgctgtgtgcgtaggtgcgccacacaattgcttccacgtagcgggacaatacagctgcggtgtcctgcagggactcgcccttacccatctgtgagctaccggaatccacgacgatggcgtgtccacccaaatgagcgatgcccgcgtcgaaggagaagcgagtacgagttgaagtcttatcaaaaagaactgcaacggactttggtccctcgagtggacgctccgaaaacggcgctgccttgagctttgcggctagggtcaaaacctctgcctgctctgcaggggtgagatcatcatcagccagaaaatggcgaacctgtggttgtgaagtcataagtttgagtcctttatgcgattgtctcggcagttcgattgtaggaaatgcaatgtgtcaagcagttaggtcaatcagagcatcggtcactcaagaattcttgagctgacagaacccaagggcgagtatggcgaattttttcaactcacgcaccaaacctactcacattggataattcgtcgagtcaactccgtgtcaacctgtcctcaactttagcggggaaacaaagatttcgcagcagatgcaaaatcatacagacacattcaaaaatggacattgaccccaaacgggggcatttccaacacttgcgctaaatagtttttgcagggaacgaacctgaaatcgttcgcactacgatctgctcccccgaatggggttaattatgaactacattacacccccgtgaatccacaaattaaagtctcagatttgaagccaatgtgatgcaactcacactggcttcaatggtaatcccaataggctcggtcgtttttaggcttcaatgcatgcctgtctggtttctcggctctcaaaccgaacaggagtgcccacggctggcaaaaagttcagttcttgggcacgctagtctggttttacctttttgagtgccccgaactgccaaacgagagtgcccatgaggggctaaatctggatccgcccgcacaaaacaactgccgcagcacaacgaatcaacgctgtgctgcggcagtcacttttttgaaggattaacgccaacccatggtcatgagtaggccgatgatcatcagaccgaagccgatgccatagttccatgcaccaagatcagccatgaatgggatctgtgggcccacgaggtagttaatgatcaaccaggctaggccgacgatcatgaaggcaaacatgatgaccttgtaccacattggggttccggcggaattgatcttaaccggggtgcggtttgcgcttgggttgcttgaaaccggtgcggtctcgtttttagttactcttgcctttggcattgttcacaccttcataatttattgtcttggaaaaaccccagcaactggtacttatcttgtttgagcttaacgtatcagttgccgggccagctaccttgacggtttccttgttggctattgcacgagtgcagcgagatcgaattcgaagagtcgcacttgcacttgggcgtctttgcggaagagggttgcaggggttggggattggaatccgattttgttttgatccacgagggctgcggtgtggatggggtcgccgacgatcagggattgatctggggcggtccagccagcagcgcggagggcactgatggcctgttcggtgaagcttggcactggccgtcgttttacaacgtcgtgactgggaaaaccctggcgttacccaacttaatcgc

SEQ ID NO: 34 - DNA, 18 bp, synthetic construct
  source 1..18: mol_type = other DNA
tccggctcgtatgttgtg

SEQ ID NO: 35 - DNA, 21 bp, synthetic construct
  source 1..21: mol_type = other DNA
tggcactggccgtcgttttac

SEQ ID NO: 36 - DNA, 6544 bp, synthetic construct
  source 1..6544: mol_type = other DNA
cgataagctagcttcacgctgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgctagaggatcgatcctttttaacccatcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaaaagtaaatcgcgcgggtttgttactgataaagcaggcaagacctaaaatgtgtaaagggcaaagtgtatactttggcgtcaccccttacatattttaggtctttttttattgtgcgtaactaacttgccatcttcaaacaggagggctggaagaagcagaccgctaacacagtacataaaaaaggagacatgaacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagtttctgaatttgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccgagcttcctgctgaacatcaaaggcaagaaaacatctgttgtcaaagacagcatccttgaacaaggacaattaacagttaacaaataaaaacgcaaaagaaaatgccgatgggtaccgagcgaaatgaccgaccaagcgacgcccaacctgccatcacgagatttcgattccaccgccgccttctatgaaaggttgggcttcggaatcgttttccgggacgccctcgcggacgtgctcatagtccacgacgcccgtgattttgtagccctggccgacggccagcaggtaggccgacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgccccgctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgctgcttccctgctgttttgtggaatatctaccgactggaaacaggcaaatgcaggaaattactgaactgaggggacaggcgagagacgatgccaaagagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagggctggatgatcctccagcgcggggatctcatgctggagttcttcgcccaccccaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagaggatccccatttgctgaaggtgttactctgcctggcccaattcctgcgggcgaagaagtgaaaaaccctgaaccttttcagaagtaactaaggccgcaatccctcgattgctgcatcaacgacggcgtctgtgagtctagctagagatctagattccaggcgccatcgttgccaatacatcggtgtgtcaatgggtatctcatcgaggaggatcacttctcctgcttttagcatgggagcagcttgggtttcgggaagaagtccccaaccaaggcgcgtttgtgtttatcggcggcgtcggcgcgcaatacggcgacaccggacggtggattttcgccgctggcgcgttcgcggcaagcctgatctggttcccgctggtgggtttcggcgcagcagcattgtcacgcccgctgtccagccccaaggtgtggcgctggatcaacgtcgtcgtggcagttgtgatgaccgcattggccatcaaactgatgttgatgggttagttttcgcgggttttggaatcggtggccttcgcccaaatgttgatgccggcgtcgtgggaaatctcatcgatcgcctccaactcggcgtcagaaaactccaagttgttgagtgaatcaaggctgttgtccagctgctcaactgacgaagcaccaatcaatgcactggtcacggtatccgcgccgtactctccttgctcgcgcagcacccatgcaagcgccatctgcgcaagtgactgcccgcgttcctgggcgatgtcattgagcttgcggaccatatcaatattgttcacgttcaacatgccctcagacagggacttaccctggctggcgcgggaaccctctggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcg

SEQ ID NO: 37 - DNA, 46 bp, synthetic construct
  source 1..46: mol_type = other DNA
ggaaacagctatgacatgattacgcggttatcgcggaatccgtatg

SEQ ID NO: 38 - DNA, 25 bp, synthetic construct
  source 1..25: mol_type = other DNA
ttaagcgttttgtgcaactccgtct

SEQ ID NO: 39 - DNA, 50 bp, synthetic construct
  source 1..50: mol_type = other DNA
agacggagttgcacaaaacgcttaaaccctacttagctgccaattattcc

SEQ ID NO: 40 - DNA, 50 bp, synthetic construct
  source 1..50: mol_type = other DNA
ggtaggtggtggtgtctttactcatgggtaaaaaatcctttcgtaggttt

SEQ ID NO: 41 - DNA, 25 bp, synthetic construct
  source 1..25: mol_type = other DNA
atgagtaaagacaccaccacctacc

SEQ ID NO: 42 - DNA, 46 bp, synthetic construct
  source 1..46: mol_type = other DNA
gttgtaaaacgacggccagtgccaccggtgatgtggttcttcactg

SEQ ID NO: 43 - DNA, 1146 bp, Moorea producens
  source 1..1146: mol_type = genomic DNA
atgtcggaaaaaattgttaattcctggaatgaatgggatgaattggaagaaatggtggtaggaattgcagactatgctagctttgaaccaaaagaaccagggaatcatccgaaattaagaaatcaaaatttagcggaaatcattcctttccccagtggacctaaagaccctaaagtccttgaaaaagctaatgaagaattaaatggactggcttatttattaaaagaccacgatgtgatagtaagaagacccgaaaaaattgattttactaaatctctaaaaacaccttactttgaagttgcaaatcaatactgtggagtctgtcctcgggatgtcatgattacctttgggaatgaaatcatggaagcgactatgtcgaagagagctagattttttgaatacttaccttaccggaaattggtctatgaatattggaataaagacgagcatatgatttggaatgctgcgcctaaaccgactatgcaggatagtatgtatctagagaatttctgggagctgtctttagaagaacgatttaagcgtatgcatgattttgaattttgtattacacaagatgaagtaatttttgatgcggctgactgtagcagattaggaaaggatatattagttcaggaatcgatgacaacaaatagaacaggaattcggtggttaaaaaagcacctagaaccaagaggatttcgggttcaccctgttcattttccccttgattttttcccctcacacattgactgtacgtttgttcctttgcgaccaggtcttattttgacaaaccctgaaagacctatacgggaagaggaggagaagatttttaaagagaatggctgggagttgatcacagttcctcaaccgacttgctcgaatgatgaaatgccaatgttttgccagtccagtaagtggttgtcaatgaatgttctgagtatatcaccgacaaaggttatctgtgaggaaagagaaaaacctctccaagaattgttggataagcatggatttgaggtttttcctttaccctttagacatgtctttgaatttggggggtcttttcattgtgcaacttgggatattcgccgaaaaggtgagtgtgaagattatttaccaaatttaaactatcaaccgatttgtggttaa

SEQ ID NO: 44 - protein, 1143 aa, Moorea producens
METSERGLULYSILEVALASNSERTRPASNGLUTRPASPGLULEUGLUGLUMETVALVALGLYILEALAASPTYRALASERPHEGLUPROLYSGLUPROGLYASNHISPROLYSLEUARGASNGLNASNLEUALAGLUILEILEPROPHEPROSERGLYPROLYSASPPROLYSVALLEUGLULYSALAASNGLUGLULEUASNGLYLEUALATYRLEULEULYSASPHISASPVALILEVALARGARGPROGLULYSILEASPPHETHRLYSSERLEULYSTHRPROTYRPHEGLUVALALAASNGLNTYRCYSGLYVALCYSPROARGASPVALMETILETHRPHEGLYASNGLUILEMETGLUALATHRMETSERLYSARGALAARGPHEPHEGLUTYRLEUPROTYRARGLYSLEUVALTYRGLUTYRTRPASNLYSASPGLUHISMETILETRPASNALAALAPROLYSPROTHRMETGLNASPSERMETTYRLEUGLUASNPHETRPGLULEUSERLEUGLUGLUARGPHELYSARGMETHISASPPHEGLUPHECYSILETHRGLNASPGLUVALILEPHEASPALAALAASPCYSSERARGLEUGLYLYSASPILELEUVALGLNGLUSERMETTHRTHRASNARGTHRGLYILEARGTRPLEULYSLYSHISLEUGLUPROARGGLYPHEARGVALHISPROVALHISPHEPROLEUASPPHEPHEPROSERHISILEASPCYSTHRPHEVALPROLEUARGPROGLYLEUILELEUTHRASNPROGLUARGPROILEARGGLUGLUGLUGLULYSILEPHELYSGLUASNGLYTRPGLULEUILETHRVALPROGLNPROTHRCYSSERASNASPGLUMETPROMETPHECYSGLNSERSERLYSTRPLEUSERMETASNVALLEUSERILESERPROTHRLYSVALILECYSGLUGLUARGGLULYSPROLEUGLNGLULEULEUASPLYSHISGLYPHEGLUVALPHEPROLEUPROPHEARGHISVALPHEGLUPHEGLYGLYSERPHEHISCYSALATHRTRPASPILEARGARGLYSGLYGLUCYSGLUASPTYRLEUPROASNLEUASNTYRGLNPROILECYSGLY

SEQ ID NO: 45 - DNA, 8312 bp, synthetic construct
  source 1..8312: mol_type = other DNA
gacagtaagacgggtaagcctgttgatgataccgctgccttactgggtgcattagccagtctgaatgacctgtcacgggataatccgaagtggtcagactggaaaatcagagggcaggaactgcagaacagcaaaaagtcagatagcaccacatagcagacccgccataaaacgccctgagaagcccgtgacgggcttttcttgtattatgggtagtttccttgcatgaatccataaaaggcgcctgtagtgccatttacccccattcactgccagagccgtgagcgcagcgaactgaatgtcacgaaaaagacagcgactcaggtgcctgatggtcggagacaaaaggaatattcagcgatttgcccgagcttgcgagggtgctacttaagcctttagggttttaaggtctgttttgtagaggagcaaacagcgtttgcgacatccttttgtaatactgcggaactgactaaagtagtgagttatacacagggctgggatctattctttttatctttttttattctttctttattctataaattataaccacttgaatataaacaaaaaaaacacacaaaggtctagcggaatttacagagggtctagcagaatttacaagttttccagcaaaggtctagcagaatttacagatacccacaactcaaaggaaaaggactagtaattatcattgactagcccatctcaattggtatagtgattaaaatcacctagaccaattgagatgtatgtctgaattagttgttttcaaagcaaatgaactagcgattagtcgctatgacttaacggagcatgaaaccaagctaattttatgctgtgtggcactactcaaccccacgattgaaaaccctacaaggaaagaacggacggtatcgttcacttataaccaatacgctcagatgatgaacatcagtagggaaaatgcttatggtgtattagctaaagcaaccagagagctgatgacgagaactgtggaaatcaggaatcctttggttaaaggctttgagattttccagtggacaaactatgccaagttctcaagcgaaaaattagaattagtttttagtgaagagatattgccttatcttttccagttaaaaaaattcataaaatataatctggaacatgttaagtcttttgaaaacaaatactctatgaggatttatgagtggttattaaaagaactaacacaaaagaaaactcacaaggcaaatatagagattagccttgatgaatttaagttcatgttaatgcttgaaaataactaccatgagtttaaaaggcttaaccaatgggttttgaaaccaataagtaaagatttaaacacttacagcaatatgaaattggtggttgataagcgaggccgcccgactgatacgttgattttccaagttgaactagatagacaaatggatctcgtaaccgaacttgagaacaaccagataaaaatgaatggtgacaaaataccaacaaccattacatcagattcctacctacataacggactaagaaaaacactacacgatgctttaactgcaaaaattcagctcaccagttttgaggcaaaatttttgagtgacatgcaaagtaagcatgatctcaatggttcgttctcatggctcacgcaaaaacaacgaaccacactagagaacatactggctaaatacggaaggatctgaggttcttatggctcttgtatctatcagtgaagcatcaagactaacaaacaaaagtagaacaactgttcaccgttacatatcaaagggaaaactgtccatatgcacagatgaaaacggtgtaaaaaagatagatacatcagagcttttacgagtttttggtgcattcaaagctgttcaccatgaacagatcgacaatgtaacagatgaacagcatgtaacacctaatagaacaggtgaaaccagtaaaacaaagcaactagaacatgaaattgaacacctgagacaacttgttacagctcaacagtcacacatagacagcctgaaacaggcgatgctgcttatcgaatcaaagctgccgacaacacgggagccagtgacgcctcccgtggggaaaaaatcatggcaattctggaagaaatagcgccattcgccattcaggctgcctgcagggaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatgggcctgcaggcctcagcgcgattcaggcctggtatgagtcagcaacaccttcttcacgaggcagatctcagcgcccccccccccctagcttgtctacgtctgatgctttgaatcggacggacttgccgatcttgtatgcggtgatttttccctcgtttgcccactttttaatggtggccggggtgagagctacgcgggcggcgacctgctgcgctgtgatccaatattcggggtcgttcactggttcccctttctgatttctggcatagaagaacccccgtgaactgtgtggttccgggggttgctgatttttgcgagacttctcgcgcaattccctagcttaggtgaaaacaccatgaaacactagggaaacacccatgaaacacccattagggcagtagggcggcttcttcgtctagggcttgcatttgggcggtgatctggtctttagcgtgtgaaagtgtgtcgtaggtggcgtgctcaatgcactcgaacgtcacgtcatttaccgggtcacggtgggcaaagagaactagtgggttagacattgttttcctcgttgtcggtggtggtgagcttttctagccgctcggtaaacgcggcgatcatgaactcttggaggttttcaccgttctgcatgcctgcgcgcttcatgtcctcacgtagtgccaaaggaacgcgtgcggtgaccacgacgggcttagcctttgcctgcgcttctagtgcttcgatggtggcttgtgcctgcgcttgctgcgcctgtagtgcctgttgagcttcttgtagttgctgttctagctgtgccttggttgccatgctttaagactctagtagctttcctgcgatatgtcatgcgcatgcgtagcaaacattgtcctgcaactcattcattatgtgcagtgctcctgttactagtcgtacatactcatatttacctagtctgcatgcagtgcatgcacatgcagtcatgtcgtgctaatgtgtaaaacatgtacatgcagattgctgggggtgcagggggcggagccaccctgtccatgcggggtgtggggcttgccccgccggtacagacagtgagcaccggggcacctagtcgcggataccccccctaggtatcggacacgtaaccctcccatgtcgatgcaaatctttaacattgagtacgggtaagctggcacgcatagccaagctaggcggccaccaaacaccactaaaaattaatagttcctagacaagacaaacccccgtgcgagctaccaactcatatgcacgggggccacataacccgaaggggtttcaattgacaaccatagcactagctaagacaacgggcacaacacccgcacaaactcgcactgcgcaaccccgcacaacatcgggtctaggtaacactgaaatagaagtgaacacctctaaggaaccgcaggtcaatgagggttctaaggtcactcgcgctagggcgtggcgtaggcaaaacgtcatgtacaagatcaccaatagtaaggctctggcggggtgccataggtggcgcagggacgaagctgttgcggtgtcctggtcgtctaacggtgcttcgcagtttgagggtctgcaaaactctcactctcgctgggggtcacctctggctgaattggaagtcatgggcgaacgccgcattgagctggctattgctactaagaatcacttggcggcgggtggcgcgctcatgatgtttgtgggcactgttcgacacaaccgctcacagtcatttgcgcaggttgaagcgggtattaagactgcgtactcttcgatggtgaaaacatctcagtggaagaaagaacgtgcacggtacggggtggagcacacctatagtgactatgaggtcacagactcttgggcgaacggttggcacttgcaccgcaacatgctgttgttcttggatcgtccactgtctgacgatgaactcaaggcgtttgaggattccatgttttcccgctggtctgctggtgtggttaaggccggtatggacgcgccactgcgtgagcacggggtcaaacttgatcaggtgtctacctggggtggagacgctgcgaaaatggcaacctacctcgctaagggcatgtctcaggaactgactggctccgctactaaaaccgcgtctaaggggtcgtacacgccgtttcagatgttggatatgttggccgatcaaagcgacgccggcgaggatatggacgctgttttggtggctcggtggcgtgagtatgaggttggttctaaaaacctgcgttcgtcctggtcacgtggggctaagcgtgctttgggcattgattacatagacgctgatgtacgtcgtgaaatggaagaagaactgtacaagctcgccggtctggaagcaccggaacgggtcgaatcaacccgcgttgctgttgctttggtgaagcccgatgattggaaactgattcagtctgatttcgcggttaggcagtacgttctagattgcgtggataaggctaaggacgtggccgctgcgcaacgtgtcgctaatgaggtgctggcaagtctgggtgtggattccaccccgtgcatgatcgttatggatgatgtggacttggacgcggttctgcctactcatggggacgctactaagcgtgatctgaatgcggcggtgttcgcgggtaatgagcagactattcttcgcacccactaaaagcggcataaaccccgttcgatattttgtgcgatgaatttatggtcaatgtcgcgggggcaaactatgatgggtcttgttgttgacaatggctgatttcatcaggaatggaactgtcatgctgttatgtgcctggctcctaatcaaagctggggacaatgggttgccccgttgatctgatctagttcggattggcggggcttcactgtatctgggggtggcatcgtgaatagattgcacaccgtagtgggcagtgtgcacaccatagtggccatgagcaccaccacccccagggacgccgacggcgcgaagctctgcgcctggtgcggctcggagatcaagcaatccggcgtcggccggagccgggactactgccgccgctcctgccgccagcgggcgtacgaggcccggcgccagcgcgaggcgatcgtgtccgccgtggcgtcggcagtcgctcgccgagatacgtcacgtgacgaaatgcagcagccttccattccgtcacgtgacgaaactcgggccgcaggtcagagcacggttccgcccgctccggccctgccggacccccggcatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatccgagtccaatgtgtccgtgttcacctcagcggccgcaccggtcggggtaccttgacaaatactttccaatggggtacaatggcgcagtgccatccactccgaggttgataccagcctcctcggtggaatgatcatccgcgtcggagacgaagtaattgacggcagcacctcgggcaaactcgagcgtctgcgggcaagcttcgcataaagacacgacgaattagacaacattagtaatgctggaagaaacaaccgagagcaggaagaacatgtcagaaaagattgttaactcctggaacgaatgggacgaacttgaagaaatggtggttggcatcgcggactacgcttccttcgagccaaaagaaccaggtaaccaccctaagctacgcaaccagaacctggccgagatcatcccattcccatccggcccaaaggacccaaaggttctcgaaaaagcaaacgaagagctgaacggcctcgcatacctcctaaaggaccacgatgttatcgtccgccgcccagaaaaaattgatttcactaagtcccttaaaaccccatacttcgaggtcgctaaccagtactgcggcgtttgcccacgcgacgttatgattaccttcggtaatgagattatggaggctaccatgagcaagcgtgcacgcttcttcgagtacttgccataccgtaagctcgtctacgagtactggaacaaggatgagcatatgatctggaacgctgctccaaagcctacgatgcaggactccatgtacttggagaacttctgggagctgtccctcgaggagcgcttcaagcgcatgcacgacttcgaattttgcattacccaggacgaggttatcttcgacgcagctgactgctcccgcctgggcaaggacatcctggtccaggagtccatgaccaccaaccgcaccggcattcgttggttgaagaagcacctcgaacctcgcggtttccgcgttcacccagtccacttccctctcgacttcttcccttctcacattgactgcactttcgtccctctccgtcccggcctcatccttaccaacccagagcgcccaatccgcgaagaagaagaaaagatcttcaaggagaacggctgggagcttatcaccgtcccacaacctacctgctccaacgacgaaatgcctatgttctgccagtcctcgaagtggctcagcatgaacgtcctttctatctccccaacaaaggttatttgcgaagagcgtgaaaagccactacaggaactcctggacaagcacggcttcgaagtcttcccactcccattccgccacgttttcgaattcggcggctcctttcactgcgccacttgggacatccgccgcaagggcgaatgcgaagactacctcccaaacctgaactaccagcctatctgcggctaataagctcggtacccggggatccgacgtcgcgttaacttaaggtaccgaattctaagcttcacaaaaaaaaaccccgcccctgacagggcggggttttttttactcagcatgtggatggccagtggtcggaccgagcgctttaattaattagcatggtgacacaagcacagtat

SEQ ID NO: 46 - DNA, 9125 bp, synthetic construct
  source 1..9125: mol_type = other DNA
cgataagctagcttcacgctgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgctagaggatcgatcctttttaacccatcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaaaagtaaatcgcgcgggtttgttactgataaagcaggcaagacctaaaatgtgtaaagggcaaagtgtatactttggcgtcaccccttacatattttaggtctttttttattgtgcgtaactaacttgccatcttcaaacaggagggctggaagaagcagaccgctaacacagtacataaaaaaggagacatgaacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagtttctgaatttgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccgagcttcctgctgaacatcaaaggcaagaaaacatctgttgtcaaagacagcatccttgaacaaggacaattaacagttaacaaataaaaacgcaaaagaaaatgccgatgggtaccgagcgaaatgaccgaccaagcgacgcccaacctgccatcacgagatttcgattccaccgccgccttctatgaaaggttgggcttcggaatcgttttccgggacgccctcgcggacgtgctcatagtccacgacgcccgtgattttgtagccctggccgacggccagcaggtaggccgacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgctgcttccctgctgttttgtggaatatctaccgactggaaacaggcaaatgcaggaaattactgaactgaggggacaggcgagagacgatgccaaagagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagggctggatgatcctccagcgcggggatctcatgctggagttcttcgcccaccccaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacggacggcgaattcctcctcgttgccacctccggcggctacggcaagcgcaccccacttgaggattactccacccagggccgtggtggcctcggcgtggtgaccttcaagtacaccccgaagcgcggtcgcctcgtcagcgccatcgcagttgaggaagatgacgagatcttcgccatcacctccgccggcggcgttgttcgcaccgaagtcaagcagatccgaccatcctcccgtgcaacaatgggtgttcgactggtcaacttggaagaaggtgtagaactgcttgccatcgacaagaacgtcgaagaccagggcgaagcatccgcagaagcagtagcaaagggtgcagtcgaaggaccagcatccaagactgctgccgaagaaaccgactccgttgacaacggatccgacgaaaacggcgaggaataatttatggcatcccgagaagtatccatcacccgaatctcaccactagcaaccttccgagttgcactggcaatgtccatcatcggactcgtggcgtggatcatctgcgtaaccgtcctctatttcggacttaacgtggctggcgtctggcagaacttcaacgatgtcatcggcggtgttggtgcagaacaaaccatcaccttcgggctcgtcctgagcatttccgcacttctgggagctatcggagcgattaccgtcgctgtgcttgcaccattgtgtgcaatcatctacaactcgattgttgacctcttcggtggactgcagattcaactgcaagaagaagtagactaacctctgaaacacaaacccctagacactgctaaatatagcctttgaacaggggatttgttgtattgttaacttgaaggtagagtcttatctcgttcctagaggggcctatagctcagtcggttagagcgcatcgctgataacgatgaggtcgcaagttcgattcttgctaggcccaccaggaacaaaaaaggggcattagctcagttggtagagcacctgctttgcaagcaggatgtcaggaggaccggtcggggtaccttgacaaatactttccaatggggtacaatggcgcagtgccatccactccgaggttgataccagcctcctcggtggaatgatcatccgcgtcggagacgaagtaattgacggcagcacctcgggcaaactcgagcgtctgcgggcaagcttcgcataaagacacgacgaattagacaacattagtaatgctggaagaaacaaccgagagcaggaagaacatgtcagaaaagattgttaactcctggaacgaatgggacgaacttgaagaaatggtggttggcatcgcggactacgcttccttcgagccaaaagaaccaggtaaccaccctaagctacgcaaccagaacctggccgagatcatcccattcccatccggcccaaaggacccaaaggttctcgaaaaagcaaacgaagagctgaacggcctcgcatacctcctaaaggaccacgatgttatcgtccgccgcccagaaaaaattgatttcactaagtcccttaaaaccccatacttcgaggtcgctaaccagtactgcggcgtttgcccacgcgacgttatgattaccttcggtaatgagattatggaggctaccatgagcaagcgtgcacgcttcttcgagtacttgccataccgtaagctcgtctacgagtactggaacaaggatgagcatatgatctggaacgctgctccaaagcctacgatgcaggactccatgtacttggagaacttctgggagctgtccctcgaggagcgcttcaagcgcatgcacgacttcgaattttgcattacccaggacgaggttatcttcgacgcagctgactgctcccgcctgggcaaggacatcctggtccaggagtccatgaccaccaaccgcaccggcattcgttggttgaagaagcacctcgaacctcgcggtttccgcgttcacccagtccacttccctctcgacttcttcccttctcacattgactgcactttcgtccctctccgtcccggcctcatccttaccaacccagagcgcccaatccgcgaagaagaagaaaagatcttcaaggagaacggctgggagcttatcaccgtcccacaacctacctgctccaacgacgaaatgcctatgttctgccagtcctcgaagtggctcagcatgaacgtcctttctatctccccaacaaaggttatttgcgaagagcgtgaaaagccactacaggaactcctggacaagcacggcttcgaagtcttcccactcccattccgccacgttttcgaattcggcggctcctttcactgcgccacttgggacatccgccgcaagggcgaatgcgaagactacctcccaaacctgaactaccagcctatctgcggctaataagctcggtacccggggatccgcctccacagttttcccggttcatcttgtttaaggtgggccgggatttttgttttttagtgtttctgggatacctcggtggcactccggcggcactctgggtcattggaaagctgtgtgtactttcgacaacaagatttgggcaaaatggtggtcaaaatcaggaaatcttgttgtcgaaagtacaactgggtcttcggttctatccgaggtgggtgagtagggtgcctgcaagtgcgatactggtgacaagtgtcgtggatccgaaagccagaatgaaaggcttgaccccaacatggatcaggttctggatttttacgccacacccgagaccaaacattgctgcagagagcaaggctgtctgtaggaaacctccagccgcaattacctcgtctgggagcgcaacagtggagcgcaaaactaccatcgccaagaatccaaggataaataggggaacgaccggtggtctctttccatcgggggacgtgtaaccctggcggcgctggcgaaaacttaaaatggcagcaatgggtgcaagtaggagtactcgggcgagtttcaccacaactgcaacacctaatgctccaccaccaatgactcctccagctgctactacttgggcgatttcatggatggatccgcctgcccacatcccaccgatttcaggggataaccccaagactttggttgcgaatgggataaaaggaatcatcagcgttccgaaaataacaacaagtgcaaccgcagtaacgacctcttcttcttcggaatcagttactccttcaacacctgccacggccgcagcgccacaaatagaaaagccacaagcaatcaacagaacttgggttggtttcattctgagcaggtgtcccatgaggatggtcccaaaaataccaccggcaacgatgcacacaatcaccgccagcatggggaaaccaagtgacaaaatatctgagaaaactaactgcagaccaaggaagacgatgaattcgagctcggtacccggggatcctctagagtcgacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgg

SEQ ID NO: 47 - DNA, 42 bp, synthetic construct
  source 1..42: mol_type = other DNA
acagctatgacatgattacggacggcgaattcctcctcgttg

SEQ ID NO: 48 - DNA, 30 bp, synthetic construct
  source 1..30: mol_type = other DNA
ggcgcgcctcctgacatcctgcttgcaaag

SEQ ID NO: 49 - DNA, 47 bp, synthetic construct
  source 1..47: mol_type = other DNA
gcaggatgtcaggaggcgcgcctccacagttttcccggttcatcttg

SEQ ID NO: 50 - DNA, 50 bp, synthetic construct
  source 1..50: mol_type = other DNA
atccccgggtaccgagctcgaattcatcgtcttccttggtctgcagttag

SEQ ID NO: 51 - DNA, 45 bp, synthetic construct
  source 1..45: mol_type = other DNA
gcaagcaggatgtcaggaggaccggtcggggtaccttgacaaata

SEQ ID NO: 52 - DNA, 47 bp, synthetic construct
  source 1..47: mol_type = other DNA
aaccgggaaaactgtggaggcggatccccgggtaccgagcttattag